SEQUENCE LISTING (532 sequences)

SEQ ID NO: 1 - protein, 394 aa, synthetic construct
  REGION 1..394: note = DEDDLI-RR
MGDKHHHHHHHHHHKDGSDKGSWEEWNARWDEWENDWNDWREDWQAWRDDWARWRATWRRGRLLSRLERLERRNEELRRLLQLIRHENRMVLQFVRALSMQNAELERRLEELARGMAQVINTNSLSLLTQNNLNRSQSALGTAIERLSSGLRINSARDDAAGQAIANRFTANIRGLTQASRNANDGISIAQTTEGALNEINNNLQRVRELAVQSANSTNSQSDLDSIQAEITQRLNEIDRVSGQTQFNGVRVLAQDNTLTIQVGANDGETIDIDLRQINSQTLGLDQLNVQQKYKDGDKGDDKTENPLQRIDAALAQVDALRSDLGAVQNRFNSAITNLGNTVNNLSEARSRIEDSDYATEVSNMSRAQILQQAGTSVLAQANQVPQNVLSLLR

SEQ ID NO: 2 - protein, 22 aa, synthetic construct
  REGION 1..22: note = His-tag
MGDKHHHHHHHHHHKDGSDKGS

SEQ ID NO: 3 - protein, 36 aa, synthetic construct
  REGION 1..36: note = Pentameric coiled coil
WEEWNARWDEWENDWNDWREDWQAWRDDWARWRATW

SEQ ID NO: 4 - protein, 4 aa, synthetic construct
  REGION 1..4: note = Linker
RRGR

SEQ ID NO: 5 - protein, 50 aa, synthetic construct
  REGION 1..50: note = Trimeric coiled coil
LLSRLERLERRNEELRRLLQLIRHENRMVLQFVRALSMQNAELERRLEEL

SEQ ID NO: 6 - protein, 282 aa, synthetic construct
  REGION 1..282: note = D0D1 of flagellin
ARGMAQVINTNSLSLLTQNNLNRSQSALGTAIERLSSGLRINSARDDAAGQAIANRFTANIRGLTQASRNANDGISIAQTTEGALNEINNNLQRVRELAVQSANSTNSQSDLDSIQAEITQRLNEIDRVSGQTQFNGVRVLAQDNTLTIQVGANDGETIDIDLRQINSQTLGLDQLNVQQKYKDGDKGDDKTENPLQRIDAALAQVDALRSDLGAVQNRFNSAITNLGNTVNNLSEARSRIEDSDYATEVSNMSRAQILQQAGTSVLAQANQVPQNVLSLLR

SEQ ID NO: 7 - protein, 53 aa, synthetic construct
  REGION 1..53: note = Trp-zipper
SSNAKWDQWSSDWQTWNAKWDQWSNDWNAWRSDWQAWKDDWARWNQRWDNWAT

SEQ ID NO: 8 - protein, 9 aa, Influenza virus
IRHENRMVL

SEQ ID NO: 9 - protein, 8 aa, synthetic construct
  REGION 1..8: note = Linker replacing D2 and D3 of flagellin
DGDKGDDK

SEQ ID NO: 10 - protein, 180 aa, unidentified
  REGION 1..180: note = Salmonella typhimurium
MAQVINTNSLSLLTQNNLNRSQSALGTAIERLSSGLRINSARDDAAGQAIANRFTANIRGLTQASRNANDGISIAQTTEGALNEINNNLQRVRELAVQSANSTNSQSDLDSIQAEITQRLNEIDRVSGQTQFNGVRVLAQDNTLTIQVGANDGETIDIDLRQINSQTLGLDQLNVQQKYK

SEQ ID NO: 11 - protein, 91 aa, unidentified
  REGION 1..91: note = Salmonella typhimurium
TENPLQRIDAALAQVDALRSDLGAVQNRFNSAITNLGNTVNNLSEARSRIEDSDYATEVSNMSRAQILQQAGTSVLAQANQVPQNVLSLLR

SEQ ID NO: 12 - protein, 4 aa, synthetic construct
  REGION 1..4: note = Linker
KKGK

SEQ ID NO: 13 - DNA, 20 bp, synthetic construct
  misc_feature 1..20: note = CpG ODN 1826
  modified_base 1..20: mod_base = OTHER; note = phosphorothioate bonds
  source 1..20: mol_type = other DNA
tccatgacgttcctgacgtt

SEQ ID NO: 14 - protein, 7 aa, synthetic construct
  REGION 1..7: note = Linker
RRGRRGR

SEQ ID NO: 15 - protein, 10 aa, synthetic construct
  REGION 1..10: note = Linker
RRGRRGRRGR

SEQ ID NO: 16 - protein, 399 aa, synthetic construct
  REGION 1..399: note = 2RR
MGDKHHHHHHHHHHKDGSDKGSEEWEEWNARWDEWENDWNDWREDWQAWRDDWARWRATWRRGRRGRLLSRLERLERRNEELRRLLQLIRHENRMVLQFVRALSMQNAELERRLEELARGMAQVINTNSLSLLTQNNLNRSQSALGTAIERLSSGLRINSARDDAAGQAIANRFTANIRGLTQASRNANDGISIAQTTEGALNEINNNLQRVRELAVQSANSTNSQSDLDSIQAEITQRLNEIDRVSGQTQFNGVRVLAQDNTLTIQVGANDGETIDIDLRQINSQTLGLDQLNVQQKYKDGDKGDDKTENPLQRIDAALAQVDALRSDLGAVQNRFNSAITNLGNTVNNLSEARSRIEDSDYATEVSNMSRAQILQQAGTSVLAQANQVPQNVLSLLR

SEQ ID NO: 17 - protein, 402 aa, synthetic construct
  REGION 1..402: note = 3RR
MGDKHHHHHHHHHHKDGSDKGSEEWEEWNARWDEWENDWNDWREDWQAWRDDWARWDATWRRGRRGRRGRLLSRLERLERRNEELRRLLQLIRHENRMVLQFVRALSMQNAELERRLEELARGMAQVINTNSLSLLTQNNLNRSQSALGTAIERLSSGLRINSARDDAAGQAIANRFTANIRGLTQASRNANDGISIAQTTEGALNEINNNLQRVRELAVQSANSTNSQSDLDSIQAEITQRLNEIDRVSGQTQFNGVRVLAQDNTLTIQVGANDGETIDIDLRQINSQTLGLDQLNVQQKYKDGDKGDDKTENPLQRIDAALAQVDALRSDLGAVQNRFNSAITNLGNTVNNLSEARSRIEDSDYATEVSNMSRAQILQQAGTSVLAQANQVPQNVLSLLR

SEQ ID NO: 18 - protein, 394 aa, synthetic construct
  REGION 1..394: note = LIVELI1-RR
MGDKHHHHHHHHHHKDGSDKGSWEEWNARWDEWENDWNDWREDWQAWRDDWARWRATWRRGRLLSRLERLERRNEELRRLLQLIRHENRMVLQFVRALSMQNAELERRLEELARGMAQVINTNSLSLLTQNNLNRSQSALGTAIERLSSGLRINSARDDAAGQAIANRFTANIRGLTQASRNANDGISIAQTTEGALNEINNNLQKVKELAVQSANSTNSQSDLDSIQAEITQRLNEIDRVSGQTQFNGVRVLAQDNTLTIQVGANDGETIDIDLRQINSQTLGLDQLNVQQKYKDGDKGDDKTENPLQRIDAALAQVDALKSDLGAVQNRFNSAITNLGNTVNNLSEARSRIEDSDYATEVSNMSRAQILQQAGTSVLAQANQVPQNVAAAAR

SEQ ID NO: 19 - protein, 394 aa, synthetic construct
  REGION 1..394: note = LIVELI2-RR
MGDKHHHHHHHHHHKDGSDKGSWEEWNARWDEWENDWNDWREDWQAWRDDWARWRATWRRGRLLSRLERLERRNEELRRLLQLIRHENRMVLQFVRALSMQNAELERRLEELARGMAQVINTNSLSLLTQNNLNKSQSALGTAIERLSSGLRINSARDDAAGQAIANRFTANIKGLTQASRNANDGISIAQTTEGALNEINNNLQKVKELAVQSANSTNSQSDLDSIQAEITQRLNEIDRVSGQTQFNGVKVLAQDNTLTIQVGANDGETIDIDLRQINSQTLGLDQLNVQQKYKDGDKGDDKTENPLQKIDAALAQVDALKSDLGAVQNRFNSAITNLGNTVNNLSEARSRIEDSDYATEVSNMSRAQILQQAGTSVLAQANQVPQNVAAAAR

SEQ ID NO: 20 - protein, 394 aa, synthetic construct
  REGION 1..394: note = LIVELI1
MGDKHHHHHHHHHHKDGSDKGSWEEWNARWDEWENDWNDWREDWQAWRDDWARWRATWMGGRLLSRLERLERRNEELRRLLQLIRHENRMVLQFVRALSMQNAELERRLEELARGMAQVINTNSLSLLTQNNLNRSQSALGTAIERLSSGLRINSARDDAAGQAIANRFTANIRGLTQASRNANDGISIAQTTEGALNEINNNLQKVKELAVQSANSTNSQSDLDSIQAEITQRLNEIDRVSGQTQFNGVRVLAQDNTLTIQVGANDGETIDIDLRQINSQTLGLDQLNVQQKYKDGDKGDDKTENPLQRIDAALAQVDALKSDLGAVQNRFNSAITNLGNTVNNLSEARSRIEDSDYATEVSNMSRAQILQQAGTSVLAQANQVPQNVAAAAR

SEQ ID NO: 21 - protein, 394 aa, synthetic construct
  REGION 1..394: note = LIVELI2
MGDKHHHHHHHHHHKDGSDKGSWEEWNARWDEWENDWNDWREDWQAWRDDWARWRATWMGGRLLSRLERLERRNEELRRLLQLIRHENRMVLQFVRALSMQNAELERRLEELARGMAQVINTNSLSLLTQNNLNKSQSALGTAIERLSSGLRINSARDDAAGQAIANRFTANIKGLTQASRNANDGISIAQTTEGALNEINNNLQKVKELAVQSANSTNSQSDLDSIQAEITQRLNEIDRVSGQTQFNGVKVLAQDNTLTIQVGANDGETIDIDLRQINSQTLGLDQLNVQQKYKDGDKGDDKTENPLQKIDAALAQVDALKSDLGAVQNRFNSAITNLGNTVNNLSEARSRIEDSDYATEVSNMSRAQILQQAGTSVLAQANQVPQNVAAAAR

SEQ ID NO: 22 - protein, 453 aa, synthetic construct
  REGION 1..453: note = CC-RR
MGHHHHHHHHHHTFRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFDASGSAKFVAAWTLKAAASGSWERWNAKWDEWRNDQNDWREDWQAWRDDWAYWTLTWRRGRLYSRLARIERRVEELRRLLQLIRHENRMVLQFVRALSMQARRLEALIDYNKAALSKFKEDARGTFRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFD

SEQ ID NO: 23 - protein, 563 aa, synthetic construct
  REGION 1..563: note = CC-RR-D0D1
MGHHHHHHHHHHTFRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFDASGSAKFVAAWTLKAAASGSWERWNAKWDEWRNDQNDWREDWQAWRDDWAYWTLTWRRGRLYSRLARIERRVEELRRLLQLIRHENRMVLQFVRALSMQARRLERRLEELARGMAQVINTNSLSLLTQNNLNRSQSALGTAIERLSSGLRINSARDDAAGQAIANRFTANIRGLTQASRNANDGISIAQTTEGALNEINNNLQRVRELAVQSANSTNSQSDLDSIQAEITQRLNEIDRVSGQTQFNGVRVLAQDNTLTIQVGANDGETIDIDLRQINSQTLGLDQLNVQQKYKDGDKGDDKTENPLQRIDAALAQVDALRSDLGAVQNRFNSAITNLGNTVNNLSEARSRIEDSDYATEVSNMSRAQILQQAGTSVLAQANQVPQNVLSLLR

SEQ ID NO: 24 - protein, 191 aa, synthetic construct
  REGION 1..191: note = substituent
MGHHHHHHHHHHTFRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFDASGSAKFVAAWTLKAAASGS

SEQ ID NO: 25 - protein, 36 aa, synthetic construct
  REGION 1..36: note = oligomerization domain
WERWNAKWDEWRNDQNDWREDWQAWRDDWAYWTLTW

SEQ ID NO: 26 - protein, 43 aa, synthetic construct
  REGION 1..43: note = oligomerization domain
LYSRLARIERRVEELRRLLQLIRHENRMVLQFVRALSMQARRL

SEQ ID NO: 27 - protein, 179 aa, synthetic construct
  REGION 1..179: note = substituent
EALIDYNKAALSKFKEDARGTFRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFD

SEQ ID NO: 28 - protein, 289 aa, synthetic construct
  REGION 1..289: note = substituent
ERRLEELARGMAQVINTNSLSLLTQNNLNRSQSALGTAIERLSSGLRINSARDDAAGQAIANRFTANIRGLTQASRNANDGISIAQTTEGALNEINNNLQRVRELAVQSANSTNSQSDLDSIQAEITQRLNEIDRVSGQTQFNGVRVLAQDNTLTIQVGANDGETIDIDLRQINSQTLGLDQLNVQQKYKDGDKGDDKTENPLQRIDAALAQVDALRSDLGAVQNRFNSAITNLGNTVNNLSEARSRIEDSDYATEVSNMSRAQILQQAGTSVLAQANQVPQNVLSLLR

SEQ ID NO: 29 - protein, 10 aa, synthetic construct
  REGION 1..10: note = His-tag
HHHHHHHHHH

SEQ ID NO: 30 - protein, 159 aa, synthetic construct
  REGION 1..159: note = CelTOS
TFRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFD

SEQ ID NO: 31 - protein, 13 aa, synthetic construct
  REGION 1..13: note = PADRE
AKFVAAWTLKAAA

SEQ ID NO: 32 - protein, 15 aa, Lymphocytic choriomeningitis virus
LIDYNKAALSKFKED

SEQ ID NO: 33 - protein, 7 aa, synthetic construct
  REGION 1..7: note = alpha-helical segment
ERRLEEL

SEQ ID NO: 34 - protein, 408 aa, synthetic construct
  REGION 1..408: note = RR-SSIEF
MGDKHHHHHHHHHHKDGSDKGSWEEWNARWDEWENDWNDWREDWQAWRDDWARWRATWRRGRLLSRLERLERRNEELRRLLQLIRHENRMVLQFVRALSMQNAELERRLEELARGMAQVINTNSLSLLTQNNLNRSQSALGTAIERLSSGLRINSARDDAAGQAIANRFTANIRGLTQASRNANDGISIAQTTEGALNEINNNLQRVRELAVQSANSTNSQSDLDSIQAEITQRLNEIDRVSGQTQFNGVRVLAQDNTLTIQVGANDGETIDIDLRQINSQTLGLDQLNVQQAKFVAAWTLKAAASSIEFARLQFDDTENPLQRIDAALAQVDALRSDLGAVQNRFNSAITNLGNTVNNLSEARSRIEDSDYATEVSNMSRAQILQQAGTSVLAQANQVPQNVLSLLR

SEQ ID NO: 35 - protein, 37 aa, synthetic construct
  REGION 1..37: note = Linker replacing D2 and D3 of flagellin
QLNVQQAKFVAAWTLKAAASSIEFARLQFDDTENPLQ

SEQ ID NO: 36 - protein, 4 aa, synthetic construct
  REGION 1..4: note = Modification of pentamer
RATW

SEQ ID NO: 37 - protein, 4 aa, synthetic construct
  REGION 1..4: note = Modified sequence in Trp-zipper
NQRW

SEQ ID NO: 38 - protein, 28 aa, synthetic construct
  REGION 1..28: note = Pan DR binding CD4 epitope string
ELRRLLQLIRHENRMVLQFVRALSMQNA

SEQ ID NO: 39 - DNA, 20 bp, synthetic construct
  misc_feature 1..20: note = CpG ODN1585
  modified_base 1..2: mod_base = OTHER; note = phosphorothioate bond
  modified_base 15..20: mod_base = OTHER; note = phosphorothioate bonds
  source 1..20: mol_type = other DNA
ggggtcaacgttgagggggg

SEQ ID NO: 40 - protein, 4 aa, synthetic construct
  REGION 1..4: note = Alanines
AAAA

SEQ ID NO: 41 - protein, 4 aa, synthetic construct
  REGION 1..4: note = Linker
MGGR

SEQ ID NO: 42 - DNA, 24 bp, synthetic construct
  misc_feature 1..24: note = CpG ODN 2006 (ODN 7909)
  modified_base 1..24: mod_base = OTHER; note = phosphorothioate bonds
  source 1..24: mol_type = other DNA
tcgtcgttttgtcgttttgtcgtt

SEQ ID NO: 43 - DNA, 20 bp, synthetic construct
  misc_feature 1..20: note = ODN 2216
  modified_base 1..2: mod_base = OTHER; note = phosphorothioate bond
  modified_base 15..20: mod_base = OTHER; note = phosphorothioate bonds
  source 1..20: mol_type = other DNA
gggggacgatcgtcgggggg

SEQ ID NO: 44 - DNA, 21 bp, synthetic construct
  misc_feature 1..21: note = ODN 2336
  modified_base 1..3: mod_base = OTHER; note = phosphorothioate bonds
  modified_base 16..21: mod_base = OTHER; note = phosphorothioate bonds
  source 1..21: mol_type = other DNA
ggggacgacgtcgtggggggg

SEQ ID NO: 45 - DNA, 23 bp, synthetic construct
  misc_feature 1..23: note = ODN BW006
  modified_base 1..23: mod_base = OTHER; note = phosphorothioate bonds
  source 1..23: mol_type = other DNA
tcgacgttcgtcgttcgtcgttc

SEQ ID NO: 46 - DNA, 22 bp, synthetic construct
  misc_feature 1..22: note = ODN 2395
  modified_base 1..22: mod_base = OTHER; note = phosphorothioate bonds
  source 1..22: mol_type = other DNA
tcgtcgttttcggcgcgcgccg

SEQ ID NO: 47 - DNA, 25 bp, synthetic construct
  misc_feature 1..25: note = ODN M362
  modified_base 1..25: mod_base = OTHER; note = phosphorothioate bonds
  source 1..25: mol_type = other DNA
tcgtcgtcgttcgaacgacgttgat

SEQ ID NO: 48 - DNA, 29 bp, synthetic construct
  misc_feature 1..29: note = ODN D-SL03
  modified_base 1..29: mod_base = OTHER; note = phosphorothioate bonds
  source 1..29: mol_type = other DNA
tcgcgaacgttcgccgcgttcgaacgcgg

SEQ ID NO: 49 - DNA, 26 bp, synthetic construct
  misc_feature 1..26: note = ODN D-SL01
  misc_feature 1..26: note = modified_bands - phosphorothioate bonds
  source 1..26: mol_type = other DNA
tcgcgacgttcgcccgacgttcggta

SEQ ID NO: 50 - protein, 46 aa, synthetic construct
  REGION 1..46: note = Synthetic: 1VDF
MDLAPQMLRELQETNAALQDVRELLRQQVKEITFLKNTVMECDACG

SEQ ID NO: 51 - protein, 330 aa, synthetic construct
  REGION 1..330: note = Synthetic: 4EEB
GSHMDFEIEVMNYSIEEFREFKTTDVESVLPFRDSSTPTWINITGIHRTDVVQRVGEFFGIHPLVLEDILNVHQRPKVEFFENYVFIVLKMFTYDKNLHELESEQVSLILTKNCVLMFQEKIGDVFDPVRERIRYNRGIIRKKRADYLLYSLIDALVDDYFVLLEKIDDEIDVLEEEVLERPEKETVQRTHQLKRNLVELAATIWPLREVLSSLYRDVPPLIEKETVPYFRDVYDHTIQIADTVETFRDIVSGLLDVYLSSVSNKTNEVMKVLTIIATIFMPLTFIAGIYGMNFEYMPELRWKWGYPVVLAVMGVIAVIMVVYFKKKKWL

SEQ ID NO: 52 - protein, 330 aa, synthetic construct; identical to SEQ ID NO: 51
  REGION 1..330: note = Synthetic: 4EED

SEQ ID NO: 53 - protein, 45 aa, synthetic construct
  REGION 1..45: note = Synthetic: 3V2P
MDLAPQMLRELQETNAALQDVRELLRQQVKEITFLKNTVMECDAC

SEQ ID NO: 54 - protein, 56 aa, synthetic construct
  REGION 1..56: note = Synthetic: 2GUV
SSNAKFDQFSSDFQTFNAKFDQFSNDFNAFRSDFQAFKDDFARFNQRFDNFATKYR

SEQ ID NO: 55 - protein, 45 aa, synthetic construct; identical to SEQ ID NO: 53
  REGION 1..45: note = Synthetic: 1MZ9

SEQ ID NO: 56 - protein, 45 aa, synthetic construct; identical to SEQ ID NO: 53
  REGION 1..45: note = Synthetic: 3V2R

SEQ ID NO: 57 - protein, 45 aa, synthetic construct; identical to SEQ ID NO: 53
  REGION 1..45: note = Synthetic: 3V2Q

SEQ ID NO: 58 - protein, 45 aa, synthetic construct; identical to SEQ ID NO: 53
  REGION 1..45: note = Synthetic: 3V2N

SEQ ID NO: 59 - protein, 52 aa, synthetic construct
  REGION 1..52: note = Synthetic: 4WBA
IEKQMDRVVKEMRRQLEMIDKLTTRAIEAVELLKRIYDKLTVQTTGEIDMTK

SEQ ID NO: 60 - protein, 31 aa, synthetic construct
  REGION 1..31: note = Synthetic: 4PN8
  VARIANT 1: note = Xaa can be any naturally occurring amino acid
XGKIEQILQKIEKILQKIEWILQKIEQILQG

SEQ ID NO: 61 - protein, 31 aa, synthetic construct; identical to SEQ ID NO: 60
  REGION 1..31: note = Synthetic: 4PN8
  VARIANT 1: note = Xaa can be any naturally occurring amino acid

SEQ ID NO: 62 - protein, 46 aa, synthetic construct; identical to SEQ ID NO: 50
  REGION 1..46: note = Synthetic: 1FBM

SEQ ID NO: 63 - protein, 53 aa, synthetic construct; identical to SEQ ID NO: 7
  REGION 1..53: note = Synthetic: 1T8Z

SEQ ID NO: 64 - protein, 31 aa, synthetic construct
  REGION 1..31: note = Synthetic: 4PND
  VARIANT 1: note = Xaa can be any naturally occurring amino acid
XGNILQKIENILKKIENILWKIENILQKIEG

SEQ ID NO: 65 - protein, 53 aa, synthetic construct
  REGION 1..53: note = Synthetic: 3MIW
MIEQQMDRIVKEMRRQLEMIDKLTTREIEQIELLKRIHDNLITRPVNVIDMSM

SEQ ID NO: 66 - protein, 53 aa, synthetic construct; identical to SEQ ID NO: 65
  REGION 1..53: note = Synthetic: 3MIW

SEQ ID NO: 67 - protein, 65 aa, synthetic construct
  REGION 1..65: note = Synthetic: 3TSI
SPSSGLGSITDLLNNILSVANQIIYNSAVALPLQLDTLESTLLTAIKSLQTSDKLEQNCSWSAAL

SEQ ID NO: 68 - protein, 84 aa, synthetic construct
  REGION 1..84: note = Synthetic: 3ZDO
GDHYDDELFSDVQDIKTALAKIHEDNQKIISKLESLLLLKGEVESIKKQINRQNISISTLEGHLSSIMIAIPGLLEHHHHHHHH

SEQ ID NO: 69 - protein, 79 aa, synthetic construct
  REGION 1..79: note = Synthetic: 4C5Q
MGDHYDDELFSDVQDIKTALAKIHEDNQKIISKLESLLLLKGEVESIKKQINRQNISISTLEGHLSSIMIAIPHHHHHH

SEQ ID NO: 70 - protein, 80 aa, synthetic construct
  REGION 1..80: note = Synthetic: 4BXT
SILTFEERDTSSLSIEARLESIEEKLSMILGLLRTLNIATAGPTAARDGIRDAMIGVREELIADIIKEAKGKAAEMMEEE

SEQ ID NO: 71 - protein, 34 aa, synthetic construct
  REGION 1..34: note = Synthetic: 3R4H
  VARIANT 1: note = Xaa can be any naturally occurring amino acid
  VARIANT 34: note = Xaa can be any naturally occurring amino acid
XGELAAIKQELAAIKKELAAIKFELAAIKQGAGX

SEQ ID NO: 72 - protein, 34 aa, synthetic construct
  REGION 1..34: note = Synthetic: 3R4A
  VARIANT 1: note = Xaa can be any naturally occurring amino acid
  VARIANT 34: note = Xaa can be any naturally occurring amino acid
XGELAAIKQELAAIKKELAAIKWELAAIKQGAGX

SEQ ID NO: 73 - protein, 95 aa, synthetic construct
  REGION 1..95: note = Synthetic: 3K4T
MANLNQIQKEVSEILSDQKSMKADIKAILELLGSQNPIKESLETVAAKIVNDLTKLINDCPCNKEILEALGTQPKEQLIEQPKEKGKGLNLGKYS

SEQ ID NO: 74 - protein, 129 aa, synthetic construct
  REGION 1..129: note = Synthetic: 3F6N
MANLNQIQKEVSEILSDQKSMKADIKAILELLGSQNPIKESLETVAAKIVNDLTKLINDCPCNKEILEALGTQPKEQLIEQPKEKGKGLNLGKYSYPNYGVGNEELGSSGNPKALTWPFKAPAGWPNQF

SEQ ID NO: 75 - protein, 52 aa, synthetic construct
  REGION 1..52: note = Synthetic: 4WB4
IEKQMDRVVKEMRRQLEMIDKLTTREIEQVELLKRIYDKLTVQTTGEIDMTK

SEQ ID NO: 76 - protein, 33 aa, synthetic construct
  REGION 1..33: note = Synthetic: 4H7R
  VARIANT 1: note = Xaa can be any naturally occurring amino acid
  VARIANT 33: note = Xaa can be any naturally occurring amino acid
XGEIKAIAQEIKAIAKEIKAIAWEIKAIAQGYX

SEQ ID NO: 77 - protein, 33 aa, synthetic construct
  REGION 1..33: note = Synthetic: 4H8F
  VARIANT 1: note = Xaa can be any naturally occurring amino acid
  VARIANT 33: note = Xaa can be any naturally occurring amino acid
XGEIKAIAQEIKAIAKEIKAIAFEIKAIAQGYX

SEQ ID NO: 78 - protein, 191 aa, synthetic construct
  REGION 1..191: note = Synthetic: 3RQA
SNAMSTLGTLAPAADTELFADTLSCELRLPAGFHVTADPGSHATAETLLRSLGQVEDLRSEDSSEERGELPLLVQRMDAKLDLILALIGRLVRQSDTRLALGTVHWSVRGIRLASPHAHPPGTTGSVLLQPSDWLPELLQLPADVLASASDGQQHWLWLRFAPLGTGLQDALERHLFRLHRRQIADARRQR

SEQ ID NO: 79 - protein, 152 aa, synthetic construct
  REGION 1..152: note = Synthetic: 4LTO
GPSSPSLLRAIPGIAWIALLLLVIFYVFAVMGTKLFAQSFPEWFGTLGASMYTLFQVMTLESWSMGIARPVIEAYPWAWIYFVSFILVSSFTVLNLFIGIIIESMQSAHWEAEDAKRIEQEQRAHDERLEMLQLIRDLSSKVDRLERRSGKR

SEQ ID NO: 80 - protein, 152 aa, synthetic construct; identical to SEQ ID NO: 79
  REGION 1..152: note = Synthetic: 4LTP

SEQ ID NO: 81 - protein, 152 aa, synthetic construct; identical to SEQ ID NO: 79
  REGION 1..152: note = Synthetic: 4LTQ

SEQ ID NO: 82 - protein, 152 aa, synthetic construct
  REGION 1..152: note = Synthetic: 4LTR
GPSSPSLLRAIPGIAWIALLLLVIFYVFAVMGTKLFAQSFPEWFGTLGASMYTLFQVMTLESWSMGIARPVIEAYPWAWIYFVSFILVSSFTVLNLFIGIIIESMQSAGWEAEDAKRIEQEQRAHDERLEMLQLIRDLSSKVDRLERRSGKR

SEQ ID NO: 83 - protein, 33 aa, synthetic construct
  REGION 1..33: note = Synthetic: 2OVC
GAVDEISMMGRVVKVEKQVQSIEHKLDLLLGFY

SEQ ID NO: 84 - protein, 32 aa, synthetic construct
  REGION 1..32: note = Synthetic: 2AG3
  VARIANT 15: note = Xaa can be any naturally occurring amino acid
RMKQIEDKLEEILSXYHIENELARIKKLLGER

SEQ ID NO: 85 - protein, 34 aa, synthetic construct
  REGION 1..34: note = Synthetic: 1UNZ
  VARIANT 1: note = Xaa can be any naturally occurring amino acid
XRMKQIEDKLEEILSKLYHIENELARSKKLLGER

SEQ ID NO: 86 - protein, 34 aa, synthetic construct
  REGION 1..34: note = Synthetic: 1UO2
  VARIANT 1: note = Xaa can be any naturally occurring amino acid
XRMKQIEDKLEEILSKLYHIENELARIKKLLGER

SEQ ID NO: 87 - protein, 34 aa, synthetic construct
  REGION 1..34: note = Synthetic: 1UO1
  VARIANT 1: note = Xaa can be any naturally occurring amino acid
XRMKQIEDKLEEILSKLYHIENELARTKKLLGER

SEQ ID NO: 88 - protein, 34 aa, synthetic construct
  REGION 1..34: note = Synthetic: 1UO0
  VARIANT 1: note = Xaa can be any naturally occurring amino acid
XRMKQIEDKLEEILSKLYHIENELARAKKLLGER

SEQ ID NO: 89 - protein, 34 aa, synthetic construct
  REGION 1..34: note = Synthetic: 1UNY
  VARIANT 1: note = Xaa can be any naturally occurring amino acid
XRMKQIEDKLEEILSKLYHIENELARGKKLLGER

SEQ ID NO: 90 - protein, 34 aa, synthetic construct
  REGION 1..34: note = Synthetic: 1UNX
  VARIANT 1: note = Xaa can be any naturally occurring amino acid
XRMKQIEDKLEEILSKLYHIENESARIKKLLGER

SEQ ID NO: 91 - protein, 34 aa, synthetic construct
  REGION 1..34: note = Synthetic: 1UNW
  VARIANT 1: note = Xaa can be any naturally occurring amino acid
XRMKQIEDKLEEILSKLYHIENEGARIKKLLGER

SEQ ID NO: 92 - protein, 34 aa, synthetic construct
  REGION 1..34: note = Synthetic: 1UNV
  VARIANT 1: note = Xaa can be any naturally occurring amino acid
XRMKQIEDKTEEILSKLYHIENELARIKKLLGER

SEQ ID NO: 93 - protein, 34 aa, synthetic construct
  REGION 1..34: note = Synthetic: 1UNU
  VARIANT 1: note = Xaa can be any naturally occurring amino acid
XRMKQIEDKSEEILSKLYHIENELARIKKLLGER

SEQ ID NO: 94 - protein, 34 aa, synthetic construct
  REGION 1..34: note = Synthetic: 1UNT
  VARIANT 1: note = Xaa can be any naturally occurring amino acid
XRMKQIEDKAEEILSKLYHIENELARIKKLLGER

SEQ ID NO: 95 - protein, 34 aa, synthetic construct; identical to SEQ ID NO: 94
  REGION 1..34: note = Synthetic: 1UO5
  VARIANT 1: note = Xaa can be any naturally occurring amino acid

SEQ ID NO: 96 - protein, 34 aa, synthetic construct
  REGION 1..34: note = Synthetic: 1UO4
  VARIANT 1: note = Xaa can be any naturally occurring amino acid
XRMKQIEDKGEEILSKLYHIENELARIKKLLGER

SEQ ID NO: 97 - protein, 34 aa, synthetic construct; identical to SEQ ID NO: 96
  REGION 1..34: note = Synthetic: 1UO3
  VARIANT 1: note = Xaa can be any naturally occurring amino acid

SEQ ID NO: 98 - protein, 45 aa, synthetic construct
  REGION 1..45: note = Synthetic: 1USE
PSSSDYSDLQRVKQELLEEVKKELQKVKEEIIEAFVQELRKRGSP

SEQ ID NO: 99 - protein, 45 aa, synthetic construct
  REGION 1..45: note = Synthetic: 1USD
PSSSDYSDLQRVKQELMEEVKKELQKVKEEIIEAFVQELRKRGSP

SEQ ID NO: 100 - protein, 33 aa, synthetic construct
  REGION 1..33: note = Synthetic: 1W5L
RMKQIEDKLEEILSKGYHICNELARIKKLLGER

SEQ ID NO: 101 - protein, 33 aa, synthetic construct
  REGION 1..33: note = Synthetic: 1W5I
RMKQIEDKLEEILSKLYHIENELARIKKLLGER

SEQ ID NO: 102 - protein, 35 aa, synthetic construct
  REGION 1..35: note = Synthetic: 1RH4
  VARIANT 1: note = Xaa can be any naturally occurring amino acid
  VARIANT 35: note = Xaa can be any naturally occurring amino acid
XAALAQIKKEIAYLLAKIKAEILAALKKIKQEIAX

SEQ ID NO: 103 - protein, 35 aa, synthetic construct
  REGION 1..35: note = Synthetic: 2O6N
  VARIANT 1: note = Xaa can be any naturally occurring amino acid
  VARIANT 35: note = Xaa can be any naturally occurring amino acid
XAEIEQAKKEIAYLIKKAKEEILEEIKKAKQEIAX

SEQ ID NO: 104 - protein, 52 aa, synthetic construct; identical to SEQ ID NO: 75
  REGION 1..52: note = Synthetic: 2O1K

SEQ ID NO: 105 - protein, 52 aa, synthetic construct
  REGION 1..52: note = Synthetic: 2O1J
IETQMDRVVKEMRRQLEMIDKLTTREIEQVELLKRIYDKLTVRTADEIDMTK

SEQ ID NO: 106 - protein, 34 aa, synthetic construct
  REGION 1..34: note = Synthetic: 2CCE
  VARIANT 1: note = Xaa can be any naturally occurring amino acid
XRMKQIEDKLEEILSKLYHISNELARIKKLLGER

SEQ ID NO: 107 - protein, 49 aa, synthetic construct
  REGION 1..49: note = Synthetic: 5AL6
GGSNKSDLAALVSLVESVRHEQQQLRNLCEMILEQQQRAKEFGENLYFQ

SEQ ID NO: 108 - protein, 50 aa, synthetic construct
  REGION 1..50: note = Synthetic: 5D5Y
SQQQIAALSESLQATQQQLQALQQQCYELEKTNRLLVSEVMTLQKMVKAQ

SEQ ID NO: 109 - protein, 61 aa, synthetic construct
  REGION 1..61: note = Synthetic: 5D60
SQQQIAALSESLQATQQQLQALQQQCYELEKTNRLLVSEVMTLQKMVKAQNQASNEIINHL

SEQ ID NO: 110 - protein, 64 aa, synthetic construct
  REGION 1..64: note = Synthetic: 4BHV
MGDHYDDELFSDVQDIKTALAKIHEDNQKIISKLESLLLLKGEVESIKKQINRQNISIHHHHHH

SEQ ID NO: 111 - protein, 119 aa, synthetic construct
  REGION 1..119: note = Synthetic: 4GJW
MDSLDDKYIMPSDDFSNTFFPHDTDRLNYHADHLGDYDLETLCEESVLMGVINSIKLINLDMRLNHIEEQVKEIPKIINKLESIDRVLAKTNTALSTIEGHLVSMMIMIPGLEHHHHHH

SEQ ID NO: 112 - protein, 33 aa, synthetic construct
  REGION 1..33: note = Synthetic: 1U9H
  VARIANT 1: note = Xaa can be any naturally occurring amino acid
  VARIANT 23: note = Xaa can be any naturally occurring amino acid
XRMKQIEDKLEEILSKLYHIENXARIKKLLGER

SEQ ID NO: 113 - protein, 32 aa, synthetic construct
  REGION 1..32: note = Synthetic: 1U9G
  VARIANT 8: note = Xaa can be any naturally occurring amino acid
RMKQIEDXEEILSKLYHIENELARIKKLLGER

SEQ ID NO: 114 - protein, 33 aa, synthetic construct
  REGION 1..33: note = Synthetic: 1U9F
  VARIANT 1: note = Xaa can be any naturally occurring amino acid
  VARIANT 16: note = Xaa can be any naturally occurring amino acid
XRMKQIEDKLEEILSXYHIENELARIKKLLGER

SEQ ID NO: 115 - protein, 52 aa, synthetic construct
  REGION 1..52: note = Synthetic: 1YBK
GSIINETADDIVYRLTVIIDDRYESLKNLITLRADRLEMIINDNVSTILASI

SEQ ID NO: 116 - protein, 115 aa, synthetic construct
  REGION 1..115: note = Synthetic: 1EZJ
MENTSSMKEMATLLTSLGVIQSAQEFESSRDASYVFARRALKSANYAEMTFNVCGLILSAEKSSARKVDENKQLLKQIQESVESFRDIYKRFSEYQKEQNSLLMSNLSTLHIITD

SEQ ID NO: 117 - protein, 52 aa, synthetic construct
  REGION 1..52: note = Synthetic: 1FE6
GSIINETADDIVYRLTVIIDDRYESLKNLITLRADRLEMIINDNVSTILASG

SEQ ID NO: 118 - protein, 43 aa, synthetic construct
  REGION 1..43: note = Synthetic: 1G1J
IEKQMDRVVKEMRRQLEMIDKLTTREIEQVELLKRIYDKLTVQ

SEQ ID NO: 119 - protein, 43 aa, synthetic construct; identical to SEQ ID NO: 118
  REGION 1..43: note = Synthetic: 1G1I

SEQ ID NO: 120 - protein, 34 aa, synthetic construct; identical to SEQ ID NO: 86
  REGION 1..34: note = Synthetic: 1GCL
  VARIANT 1: note = Xaa can be any naturally occurring amino acid

SEQ ID NO: 121 - protein, 74 aa, synthetic construct
  REGION 1..74: note = Synthetic: 3SWF
GAMGLEEKVTRMESSVDLLQTRFARILAEYESMQQKLKQRLTKVEKFLKPLIDTEFSAIEGSGTESGPTDSTQD

SEQ ID NO: 122 - protein, 46 aa, synthetic construct
  REGION 1..46: note = Synthetic: 3SWY
GALEEKVEQLGSSLDTLQTRFARLLAEYNATQMKMKQRLSQLESQV

SEQ ID NO: 123 - protein, 39 aa, synthetic construct
  REGION 1..39: note = Synthetic: 3TE3
GGWVSGEEFYMLTRRVLQLETVLEGVVSQIDAVGSKLKM

SEQ ID NO: 124 - protein, 36 aa, synthetic construct
  REGION 1..36: note = Synthetic: 3TQ2
  VARIANT 1: note = Xaa can be any naturally occurring amino acid
XKVSALKEKVSALKEQFLMLMFKVSALKEKVSALKE

SEQ ID NO: 125 - protein, 40 aa, synthetic construct
  REGION 1..40: note = Synthetic: 4P67
  VARIANT 1: note = Xaa can be any naturally occurring amino acid
  VARIANT 40: note = Xaa can be any naturally occurring amino acid
XWGSIDQINGKLNRVIEKFHQIEKEFSEVEGRIQDLEKYX

SEQ ID NO: 126 - protein, 40 aa, synthetic construct; identical to SEQ ID NO: 125
  REGION 1..40: note = Synthetic: 4OXM
  VARIANT 1: note = Xaa can be any naturally occurring amino acid
  VARIANT 40: note = Xaa can be any naturally occurring amino acid

SEQ ID NO: 127 - protein, 77 aa, synthetic construct
  REGION 1..77: note = Synthetic: 3VTQ
  VARIANT 1: note = Xaa can be any naturally occurring amino acid
  VARIANT 38..39: note = Xaa can be any naturally occurring amino acid
  VARIANT 77: note = Xaa can be any naturally occurring amino acid
XSGIVQQQNNLLRAIEAQQHLLQLTVWGIKQLQARILXXMTWETWEREIENYTKQIYKILEESQEQQDRNEKDLLEX

SEQ ID NO: 128 - protein, 62 aa, synthetic construct
  REGION 1..62: note = Synthetic: 3VU5
  VARIANT 39..40: note = Xaa can be any naturally occurring amino acid
NNLLRAIEAQQHLLQLTVWGIKQLQARILAVERYLKDQXXWEEWDKKIEEYTKKIEELIKKS

SEQ ID NO: 129 - protein, 64 aa, synthetic construct
  REGION 1..64: note = Synthetic: 3VU6
  VARIANT 39..40: note = Xaa can be any naturally occurring amino acid
NNLLRAIEAQQHLLQLTVWGIKQLQARILAVERYLKDQXXMTWEEWDKKIEEYTKKIEELIKKS

SEQ ID NO: 130 - protein, 32 aa, synthetic construct
  REGION 1..32: note = Synthetic: 3W92
  VARIANT 18: note = Xaa can be any naturally occurring amino acid
RVARLEKKVSALEKKVAXLEKEVARLKKLVGE

SEQ ID NO: 131 - protein, 32 aa, synthetic construct; identical to SEQ ID NO: 130
  REGION 1..32: note = Synthetic: 3W93
  VARIANT 18: note = Xaa can be any naturally occurring amino acid

SEQ ID NO: 132 - protein, 32 aa, synthetic construct
  REGION 1..32: note = Synthetic: 3W8V
RVARLEKKVSALEKKVAGLEKEVARLKKLVGE

SEQ ID NO: 133 - protein, 51 aa, synthetic construct
  REGION 1..51: note = Synthetic: 3VYI
GSERQILRIKQNINQIATKIQHIEFSISEIEQEIERINKLIKQNGLLGDVN

SEQ ID NO: 134 - protein, 220 aa, synthetic construct
  REGION 1..220: note = Synthetic: 3NTN
YNEATIENSTVGGGGYNQAKGRNSTVAGGYNNEATGTDSTIAGGRKNQATGKGSFAAGIDNKANADNAVALGNKNTIEGENSVAIGSNNTVKKGQQNVFILGSNTDTTNAQNGSVLLGHNTAGKAATIVNSAEVGGLSLTGFAGASKTGNGTVSVGKKGKERQIVHVGAGEISDTSTDAVNGSQLHALATVVAQNKADIKDLDDEVGLLGEEINKHHHHH

SEQ ID NO: 135 - protein, 113 aa, synthetic construct
  REGION 1..113: note = Synthetic: 3ZMF
MKQIEDKIEEILSKIYHIENEIARIKKLITNTEASVAGLAEDALLWDESISAFSASHTGNASKITNLAAGTLAADSTDAVNGSQMKQIEDKIEEILSKIYHIENEIARIKKLI

SEQ ID NO: 136 - protein, 208 aa, synthetic construct
  REGION 1..208: note = Synthetic: 4CJD
MKDNPPPSTDEIAKAALVGVYNNTQDINGFKVGDTIYDIENGQPKGRPATEDDVKADDFGGLGLKEVLAQHDQSLADLTGTVEENSEALVKTAEVVNDISADVKANTAAIRENKAAIATKADKTELDEVSGKVTANETAIGKKANSADVYTKAEVYTKQESDNRFVKIGDRIGNLNTTANGLETRLADAEKSVKDHGTRASKHHHHHH

SEQ ID NO: 137 - protein, 51 aa, synthetic construct
  REGION 1..51: note = Synthetic: 4CGB
GSGMEVDDRVSALEQRLQLQEDELAVLKAALADALRRLRACEEQGAALRAR

SEQ ID NO: 138 - protein, 59 aa, synthetic construct
  REGION 1..59: note = Synthetic: 4CGC
GSLDDSISAASTSDVQDRLSALESRVQQQEDEMTVLKAALADVLRRLAISEDHVASVKK

SEQ ID NO: 139 - protein, 73 aa, synthetic construct
  REGION 1..73: note = Synthetic: 3PP5
GPLGSMSTKTNIQKDWEQREFIEDMSINIQKIVEFLNKFELSTRNKLSDLNEKLTILDRQVDYLEATFKTVQE

SEQ ID NO: 140 - protein, 311 aa, synthetic construct
  REGION 1..311: note = Synthetic: 3PR7
MATTASAQKVGKATNKISGGDNNTANGTYLTIGGGDYNKTKGRYSTIGGGLFNEATNEYSTIGSGGYNKAKGRYSTIGGGGYNEATNQYSTIGGGDNNTAKGRYSTIGGGGYNEATIENSTVGGGGYNQAKGRNSTVAGGYNNEATGTDSTIAGGRKNQATGKGSFAAGIDNKANADNAVALGNKNTIEGENSVAIGSNNTVKKGQQNVFILGSNTDTTNAQNGSVLLGHNTAGKAATIVNSAEVGGLSLTGFAGASKTGNGTVSVGKKGKERQIVHVGAGEISDTSTDAVNGSQLHALATVVAKHHHHHH

SEQ ID NO: 141 - protein, 66 aa, synthetic construct
  REGION 1..66: note = Synthetic: 3R48
  VARIANT 1: note = Xaa can be any naturally occurring amino acid
  VARIANT 34..35: note = Xaa can be any naturally occurring amino acid
  VARIANT 66: note = Xaa can be any naturally occurring amino acid
XGELKAIAQELKAIAKELKAIAWEHKAIAQGAGXXGELKAIAQELKAIAYELKAIAKEDKAIAQGX

SEQ ID NO: 142 - protein, 32 aa, synthetic construct
  REGION 1..32: note = Synthetic: 4G1A
QIAALEQKIAALEQKCAACEQKIAALEQKGGY

SEQ ID NO: 143 - protein, 487 aa, synthetic construct
  REGION 1..487: note = Synthetic: 3DUZ
AEHCNAQMKTGPYKIKNLDITPPKETLQKDVEITIVETDYNENVIIGYKGYYQAYAYNGGSLDPNTRVEETMKTLNVGKEDLLMWSIRQQCEVGEELIDRWGSDSDDCFRDNEGRGQWVKGKELVKRQNNNHFAHHTCNKSWRCGISTSKMYSRLECQDDTDECQVYILDAEGNPINVTVDTVLHRDGVSMILKQKSTFTTRQIKAACLLIKDDKNNPESVTREHCLIDNDIYDLSKNTWNCKFNRCIKRKVEHRVKKRPPTWRHNVRAKYTEGDTATKGDLMHIQEELMYENDLLKMNIELMHAHINKLNNMLHDLIVSVAKVDERLIGNLMNNSVSSTFLSDDTFLLMPCTNPPAHTSNCYNNSIYKEGRWVANTDSSQCIDFSNYKELAIDDDVEFWIPTIGNTTYHDSWKDASGWSFIAQQKSNLITTMENTKFGGVGTSLSDITSMAEGELAAKLTSFMFGHVVNFVIILIVILDYKDDDDK

SEQ ID NO: 144 - protein, 31 aa, synthetic construct
  REGION 1..31: note = Synthetic: 3H5F
  VARIANT 1: note = Xaa can be any naturally occurring amino acid
  VARIANT 17: note = Xaa can be any naturally occurring amino acid
  VARIANT 31: note = Xaa can be any naturally occurring amino acid
XEWEALEKKLAALESKXQALEKKLEALEHGX

SEQ ID NO: 145 - protein, 31 aa, synthetic construct
  REGION 1..31: note = Synthetic: 3H5G
  VARIANT 1: note = Xaa can be any naturally occurring amino acid
  VARIANT 31: note = Xaa can be any naturally occurring amino acid
XEWEALEKKLAALESKVQALEKKLEALEHGX

SEQ ID NO: 146 - protein, 61 aa, synthetic construct
  REGION 1..61: note = Synthetic: 3H7Z
HTLKTANSYTDVTVSNSTKKAIRESNQYTDHKFHQLDNRLDKLDTRLLKLLASSAALNSLL

SEQ ID NO: 147 - protein, 64 aa, synthetic construct
  REGION 1..64: note = Synthetic: 3H7X
SKSSHTLKTANSYTDVTVSNSTKKAIRESNQYTDHKFHQLDNRLDKLDTRVDKGLASSAALNSL

SEQ ID NO: 148 - protein, 31 aa, synthetic construct
  REGION 1..31: note = Synthetic: 3HFC
GSRGSNTIGARLNRVEDKVTQMDQRMALITD

SEQ ID NO: 149 - protein, 31 aa, synthetic construct
  REGION 1..31: note = Synthetic: 3HFE
GARGSNTIGARLNRVEDKVTQLDQRLALITD

SEQ ID NO: 150 - protein, 64 aa, synthetic construct
  REGION 1..64: note = Synthetic: 3HRN
MGVSYEEFQVLVRRVDRMEHSIGSIVSKIDAVIVKLEIMERAKLKRREVLGRLLDGVAEDERLG

SEQ ID NO: 151 - protein, 44 aa, synthetic construct
  REGION 1..44: note = Synthetic: 3HRO
GSHMGVSYEEFQVLVRRVDRMEHSIGSIVSKIDAVIVKLEIMER

SEQ ID NO: 152 - protein, 109 aa, synthetic construct
  REGION 1..109: note = Synthetic: 2WG5
MHHHHHHRMKQLEDKVEELLSKNYHLENEVARLRSPPLLVGVVSDILEDGRVVVKSSTGPKFVVNTSQYINEEELKPGARVALNQQTLAIVNVLPTSKDPMVYGFEVEE

SEQ ID NO: 153 - protein, 211 aa, synthetic construct
  REGION 1..211: note = Synthetic: 2VRS
ILQTTVDGNSTAISNLKSDISSNGLAITDLQDRVKSLESTASHGLSFSPPLSVADGVVSLDMDPYFCSQRVSLTSYSAEAQLMQFRWMARGTNGSSDTIDMTVNAHCHGRRTDYMMSSTGNLTVTSNVVLLTFDLSDITHIPSDLARLVPSAGFQAASFPVDVSFTRDSATHAYQAYGVYSSSRVFTITFPTGGDGTANIRSLTVRTGIDT

SEQ ID NO: 154 - protein, 56 aa, synthetic construct
  REGION 1..56: note = Synthetic: 2W6B
GPLGSKSLVDTVYALKDEVQELRQDNKKMKKSLEEEQRARKDLEKLVRKVLKNMND

SEQ ID NO: 155 - protein, 109 aa, synthetic construct
  REGION 1..109: note = Synthetic: 2WG6
MHHHHHHRMKQLEDKVEELLSKNYHLENEVARLRSAPLLVGVVSDILEDGRVVVKSSTGPKFVVNTSQYINEEELKPGARVALNQQTLAIVNVLPTSKDPMVYGFEVEE

SEQ ID NO: 156 - protein, 107 aa, synthetic construct
  REGION 1..107: note = Synthetic: 2WPS
MKQIEDKIEEILSKIYHIENEIARIKKLIEKVDQNTADITTNTNSINQNTTDIATNTTNINNLSDSITTLMKQIEDKIEEILSKIYHIENEIARIKKLIKLHHHHHH

SEQ ID NO: 157 - protein, 34 aa, synthetic construct
  REGION 1..34: note = Synthetic: 2WPY
  VARIANT 1: note = Xaa can be any naturally occurring amino acid
XRMKQLEDKVEELLSKVYHNENEVARLKKLVGER

SEQ ID NO: 158 - protein, 34 aa, synthetic construct
  REGION 1..34: note = Synthetic: 2WPZ
  VARIANT 1: note = Xaa can be any naturally occurring amino acid
XRMKQLEDKVEENLSKVYHNENEVARLKKLVGER

SEQ ID NO: 159 - protein, 33 aa, synthetic construct
  REGION 1..33: note = Synthetic: 2WQ0
RMKQLEDKIEENTSKIYHNTNEIARNTKLVGER

SEQ ID NO: 160 - protein, 33 aa, synthetic construct; identical to SEQ ID NO: 159
  REGION 1..33: note = Synthetic: 2WQ1

SEQ ID NO: 161 - protein, 33 aa, synthetic construct; identical to SEQ ID NO: 159
  REGION 1..33: note = Synthetic: 2WQ2

SEQ ID NO: 162 - protein, 107 aa, synthetic construct; identical to SEQ ID NO: 156
  REGION 1..107: note = Synthetic: 2WPR

SEQ ID NO: 163 - protein, 33 aa, synthetic construct; identical to SEQ ID NO: 159
  REGION 1..33: note = Synthetic: 2WQ3

SEQ ID NO: 164 - protein, 30 aa, synthetic construct
  REGION 1..30: note = Synthetic: 2X6P
  VARIANT 1: note = Xaa can be any naturally occurring amino acid
XEWEALEKKLAALESKLQACEKKLEALEHG

SEQ ID NO: 165 - protein, 90 aa, synthetic construct
  REGION 1..90: note = Synthetic: 3KPE
HLEGEVNKIKSALLSTNKAVVSLSNGVSVLTSKVLDLKNYIDKQLLPIVNKVFPSDEFDASISQVNEKINQSLAFIRKSDELLHNVNAGK

SEQ ID NO: 166 - protein, 65 aa, synthetic construct
  REGION 1..65: note = Synthetic: 3L35
  VARIANT 1: note = Xaa can be any naturally occurring amino acid
  VARIANT 47..48: note = Xaa can be any naturally occurring amino acid
  VARIANT 65: note = Xaa can be any naturally occurring amino acid
XRMKQIEDKIEEIESKQKKIENEIARIKKLLQLTVWGIKQLQARILXXKGHPCDYPEWQWLCELX

SEQ ID NO: 167 - protein, 64 aa, synthetic construct
  REGION 1..64: note = Synthetic: 3L36
  VARIANT 1: note = Xaa can be any naturally occurring amino acid
  VARIANT 47..48: note = Xaa can be any naturally occurring amino acid
  VARIANT 64: note = Xaa can be any naturally occurring amino acid
XRMKQIEDKIEEIESKQKKIENEIARIKKLLQLTVWGIKQLQARILXXKHPCDYPEWQWLCELX

SEQ ID NO: 168 - protein, 65 aa, synthetic construct; identical to SEQ ID NO: 166
  REGION 1..65: note = Synthetic: 3L37
  VARIANT 1: note = Xaa can be any naturally occurring amino acid
  VARIANT 47..48: note = Xaa can be any naturally occurring amino acid
  VARIANT 65: note = Xaa can be any naturally occurring amino acid

SEQ ID NO: 169 - protein, 74 aa, synthetic construct
  REGION 1..74: note = Synthetic: 3AHA
  VARIANT 1: note = Xaa can be any naturally occurring amino acid
  VARIANT 38..39: note = Xaa can be any naturally occurring amino acid
  VARIANT 74: note = Xaa can be any naturally occurring amino acid
XSDIVQQQNNLLRAIEAQQHLLQLTVWGIKQLQARILXXWMEWDREINKYTSLIHSLIEQSQNQQEKNEQELLX

SEQ ID NO: 170 - protein, 35 aa, synthetic construct
  REGION 1..35: note = Synthetic: 3GJP
GSRMKQLEDKVEELLSKNYHLENEIARIKKLVGER

SEQ ID NO: 171 - protein, 188 aa, synthetic construct
  REGION 1..188: note = Synthetic: 3BK6
MIFEKAVIVDLRTQVLDVPVQETITKDNVPVRVNAVVYFRVVDPVKAVTQVKNYIMATSQISQTTLRSVIGQAHLDELLSERDKLNMQLQRIIDEATDPWGIKVTAVEIKDVELPAGMQKAMARQAEAERERRARITLAEAERQAAEKLREAAEIISEHPMALQLRTLQTISDVAGDKSNLEHHHHHH

SEQ ID NO: 172 - protein, 505 aa, synthetic construct
  REGION 1..505: note = Synthetic: 4UW0
MTKDLNTLVSELPEIYQTIFGHPEWDGDAARDCNQRLDLITEQYDNLSRALGRPLNVLDLGCAQGFFSLSLASKGATIVGIDFQQENINVCRALAEENPDFAAEFRVGRIEEVIAALEEGEFDLAIGLSVFHHIVHLHGIDEVKRLLSRLADVTQAVILELAVKEEPLYWGVSQPDDPRELIEQCAFYRLIGEFDTHLSPVPRPMYLVSNHRVLINDFNQPFQHWQNQPYAGAGLAHKRSRRYFFGEDYVCKFFYYDMPHGILTAEESQRNKHELHNEIKFLTQPPAGFDAPAVLAHGENAQSGWLVMEKLPGRLLSDMLAAGEEIDREKILGSLLRSLAALEKKGFWHDDVRPWNVMVDARQHARLIDFGSIVTTPQDCSWPTNLVQSFFVFVNELFAENKSWNGFWRSAPVHPFNLPQPWSNWLYAVWQEPVERWNFALLLALFEKKAKLPSAEQQRGATEQWIIAQETVLLELQSRVRNESAGSEALRGQIHTLEQQMAQLQ

SEQ ID NO: 173 - protein, 703 aa, synthetic construct
  REGION 1..703: note = Synthetic: 4L1R
DPAAPTSPGTPGVAAATQAANGGPATPAPPALGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNTIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAA

SEQ ID NO: 174 - protein, 403 aa, synthetic construct
  REGION 1..403: note = Synthetic: 4JBZ
MKIEEGKLVIWINGDKGYNGLAEVGKKFEKDTGIKVTVEHPDKLEEKFPQVAATGDGPDIIFWAHDRFGGYAQSGLLAEITPAAAFQDKLYPFTWDAVRYNGKLIAYPIAVEALSLIYNKDLLPNPPKTWEEIPALDKELKAKGKSALMFNLQEPYFTWPLIAADGGYAFKYAAGKYDIKDVGVDNAGAKAGLTFLVDLIKNKHMNADTDYSIAEAAFNKGETAMTINGPWAWSNIDTSAVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDAALAAAQTNAAAMGVCQEKSKDELEDELRKMQAQMKKLQEQLQK

SEQ ID NO: 175 - protein, 219 aa, synthetic construct
  REGION 1..219: note = Synthetic: 4JDO
MGNSGFYLYNTENCVFADNIKVGQMTEPLKDQQIILGTKSTPVAAKMTASDGISLTVSNNSSTNASITIGLLKAFNNFPITNKIQCNGLFTPSNIETLLGGTEIGKFTVTPKSSGSMFLVSADIIASRMEGGVVLALVREGDSKPCAISYGYSSGVPNLCSLRTSITNTGLTPTTYSLRVGGLESGVVWVNALSNGNDILGITNTSNVSFLEVIPQTNA

SEQ ID NO: 176 - protein, 50 aa, synthetic construct
  REGION 1..50: note = Synthetic: 4R0R
  VARIANT 1: note = Xaa can be any naturally occurring amino acid
  VARIANT 50: note = Xaa can be any naturally occurring amino acid
XYGIKKEIEAIKKEQEAIKKKIEAIEKELRQLANETTQALQLFLRATTEX

SEQ ID NO: 177 - protein, 83 aa, synthetic construct
  REGION 1..83: note = Synthetic: 4YPC
VDQLTNDKARVEVERDNLAEDIMRLREKLQEEMLQREEAENTLQSFRQDVDNASLARLDLERKVESLQEEIAFLKKLHEEEIQ

SEQ ID NO: 178 - protein, 177 aa, synthetic construct
  REGION 1..177: note = Synthetic: 4E52
GSPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 179 - protein, 64 aa, synthetic construct
  REGION 1..64: note = Synthetic: 3MGN
  VARIANT 1: note = Xaa can be any naturally occurring amino acid
  VARIANT 47..48: note = Xaa can be any naturally occurring amino acid
  VARIANT 64: note = Xaa can be any naturally occurring amino acid
XRMKQIEDKIEEIESKQKKIENEIARIKKLLQLTVWGIKQLQARILXXKGFVCPPEWRWLCDLX

SEQ ID NO: 180 - protein, 137 aa, synthetic construct
  REGION 1..137: note = Synthetic: 3MKO
HHHHHHIEGRDEEFSDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMD

SEQ ID NO: 181 - protein, 251 aa, synthetic construct
  REGION 1..251: note = Synthetic: 3M9B
MGESERSEAFGIPRDSPLSSGDAAELEQLRREAAVLREQLENAVGSHAPTRSARDIHQLEARIDSLAARNSKLMETLKEARQQLLALREEVDRLGQPPSGYGVLLATHDDDTVDVFTSGRKMRLTCSPNIDAASLKKGQTVRLNEALTVVEAGTFEAVGEISTLREILADGHRALVVGHADEERVVWLADPLIAEDLPDGLPEALNDDTRPRKLRPGDSLLVDTKAGYAFERIPLVPRGSAAALEHHHHHH

SEQ ID NO: 182 - protein, 319 aa, synthetic construct
  REGION 1..319: note = Synthetic: 3M9D
MGESERSEAFGIPRDSPLSSGDAAELEQLRREAAVLREQLENAVGSHAPTRSARDIHQLEARIDSLAARNSKLMETLKEARQQLLALREEVDRLGQPPSGYGVLLATHDDDTVDVFTSGRKMRLTCSPNIDAASLKKGQTVRLNEALTVVEAGTFEAVGEISTLREILADGHRALVVGHADEERVVWLADPLIAEDLPDGLPEALNDDTRPRKLRPGDSLLVDTKAGYAFERIPLVPRGSAAALEHHHHHHGSHMMAQEQTKRGGGGGDDDDIAGSTAAGQERREKLTEETDDLLDEIDDVLEENAEDFVRAYVQKGGE

SEQ ID NO: 183 - protein, 233 aa, synthetic construct
  REGION 1..233: note = Synthetic: 2YKO
MASLRSRCDQLEERVSAAEDEINEIKREGKFREKRIKRNEQSLQEIWDYVKRPNLRLIGVPESDVENGTKLENTLQDIIQENFPNLARQANVQIQEIQRTPQRYSSRRATPRHIIVRFTKVEMKEKMLRAAREKGRVTLKGKPIRLTVDLSAETLQARREWGPIFNILKEKNFQPRISYPAKLSFISEGEIKYFIDKQMLRDFVTTRPALKELLKEALNMERNNRYQHHHHHH

SEQ ID NO: 184 - protein, 229 aa, synthetic construct
  REGION 1..229: note = Synthetic: 2YKP
MASLRSRCDQLEERVSAAEDEINEIKREGKFREKRIKRNEQSLQEIWDYVKRPNLRLIGVPESDVENGTKLENTLQDIIQENFPNLARQANVQIQEIQRTPQRYSSRRATPRHIIVRFTKVEMKEKMLRAAREKGRVTLKGKPIRLTVDLSAETLQARREWGPIFNILKEKNFQPRISYPAKLSFISEGEIKYFIDKQMLRDFVTTRPALKELLKEALNMERNHHHHHH

SEQ ID NO: 185 - protein, 229 aa, synthetic construct; identical to SEQ ID NO: 184
  REGION 1..229: note = Synthetic: 2YKQ

SEQ ID NO: 186 - protein, 106 aa, synthetic construct
  REGION 1..106: note = Synthetic: 2YNY
MKQIEDKIEEILSKIYHIENEIARIKKLIYSLSQSVADRLGGGASVNSDGTVNAPLYEVGTGIYNNVGSALSALNTSMKQIEDKIEEILSKIYHIENEIARIKKLI

SEQ ID NO: 187 - protein, 154 aa, synthetic construct
  REGION 1..154: note = Synthetic: 2YNZ
MKQIEDKIEEILSKIYHIENEIARIKKLIYETNQKVDQNTSAIADINTSITNLGTDALSWDDEEGAFSASHGTSGTNKITNVAAGEIASDSTDAVNGSQLYETNMLISQYNESISQLAGDTSETYITENGTGVKYIRTNDNGLEGQDAYATGNG

SEQ ID NO: 188 - protein, 322 aa, synthetic construct
  REGION 1..322: note = Synthetic: 2YO0
MKQIEDKIEEILSKIYHIENEIARIKKLIQNAIGAVTTTPTKYYHANSTEEDSLAVGTDSLAMGAKTIVNADAGIGIGLNTLVMADAINGIAIGSNARANHANSIAMGNGSQTTRGAQTDYTAYNMDTPQNSVGEFSVGSEDGQRQITNVAAGSADTDAVNVGQLKVTDAQVSRNTQSITNLNTQVSNLDTRVTNIENGIGDIVTTGSTKYFKTNTDGADANAQGADSVAIGSGSIAAAENSVALGTNSVADEANTVSVGSSTQQRRITNVAAGVNNTDAVNVAQMKQIEDKIEEILSKIYHIENEIARIKKLIKLHHHHHH

SEQ ID NO: 189 - protein, 322 aa, synthetic construct; identical to SEQ ID NO: 188
  REGION 1..322: note = Synthetic: 2YO1

SEQ ID NO: 190 - protein, 170 aa, synthetic construct
  REGION 1..170: note = Synthetic: 2YO2
MKQIEDKIEEILSKIYHIENEIARIKKLIYSLSQSVADRLGGGASVNSDGTVNAPLYEVGTGIYNNVGSALSALNTSITNTEASVAGLAEDALLWDESISAFSASHTGNASKITNLAAGTLAADSTDAVNGSQMKQIEDKIEEILSKIYHIENEIARIKKLIKLHHHHHH

SEQ ID NO: 191 - protein, 74 aa, synthetic construct
  REGION 1..74: note = Synthetic: 2Z2T
  VARIANT 1: note = Xaa can be any naturally occurring amino acid
  VARIANT 38..39: note = Xaa can be any naturally occurring amino acid
  VARIANT 74: note = Xaa can be any naturally occurring amino acid
XSDIVQQQNNLLRAIEAQQHLLQLTVWGIKQLQARILXXWLEWDRKIEEYTKKIEELIKKSQEQQEKNEKELKX

SEQ ID NO: 192 - protein, 31 aa, synthetic construct
  REGION 1..31: note = Synthetic: 3LJM
  VARIANT 1: note = Xaa can be any naturally occurring amino acid
  VARIANT 31: note = Xaa can be any naturally occurring amino acid
XEWEALEKKCAALESKLQALEKKLEALEHGX

SEQ ID NO: 193 - protein, 64 aa, synthetic construct
  REGION 1..64: note = Synthetic: 3LT6
KSSHTLKTANSYTDVTVSNSTKKAIRESNQYTDHKMHQLENRLDKLEKRLLKLLASSAALNSLF

SEQ ID NO: 194 - protein, 64 aa, synthetic construct
  REGION 1..64: note = Synthetic: 3LT7
KSSHTLKTANSYTDVTVSNSTKKAIRESNQYTDHKFHQLENRLDKLEKRLLKLLASSAALNSLF

SEQ ID NO: 195 - protein, 32 aa, synthetic construct
  REGION 1..32: note = Synthetic: 4DZK
GEIAAIKQEIAANKKEIAAIKWEIAAIKQGYG

SEQ ID NO: 196 - protein, 33 aa, synthetic construct
  REGION 1..33: note = Synthetic: 4DZL
  VARIANT 1: note = Xaa can be any naturally occurring amino acid
XGEIAAIKQEIAAIKKEIAAIKFEIAAIKQGYG

SEQ ID NO: 197 - protein, 33 aa, synthetic construct
  REGION 1..33: note = Synthetic: 4DZN
  VARIANT 1: note = Xaa can be any naturally occurring amino acid
XGEIAALKQEIAALKKEIAALKFEIAALKQGYY

SEQ ID NO: 198 - protein, 78 aa, synthetic construct
  REGION 1..78: note = Synthetic: 3EFG
MHEQLSPRDQELEARLVELETRLSFQEQALTELSEALADARLTGARNAELIRHLLEDLGKVRSTLFADAADEPPPPHY

SEQ ID NO: 199 - protein, 703 aa, synthetic construct
  REGION 1..703: note = Synthetic: 3NWD
DPAAPTSPGTPGVAAATQAANGGPATPAPPALGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRSSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAA

SEQ ID NO: 200 - protein, 703 aa, synthetic construct
  REGION 1..703: note = Synthetic: 3NWF
DPAAPTSPGTPGVAAATQAANGGPATPAPPALGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAA

SEQ ID NO: 201 - protein, 703 aa, synthetic construct
  REGION 1..703: note = Synthetic: 3NWA
DPAAPTSPGTPGVAAATQAANGGPATPAPPALGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVRFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAA

SEQ ID NO: 202 - protein, 157 aa, synthetic construct
  REGION 1..157: note = Synthetic: 2QIH
MRGSHHHHHHGSATSANTDRIATAELGIAENKKDAQIAKAQANENKDGIAKNQADIQLHDKKITNLGILHSMVARAVGNNTQGVATNKADIAKNQADIANNIKNIYELAQQQDQHSSDIKTLAKVSAANTDRIAKNKAEADASFETLTKNQKLCSQA

SEQ ID NO: 203 - protein, 166 aa, synthetic construct
  REGION 1..166: note = Synthetic: 2R32
GSHMGGSRMKQIEDKIEEILSKIYHIENEIARIKKLIGERETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 204 - protein, 68 aa, synthetic construct
  REGION 1..68: note = Synthetic: 2OT5
SGIVQQQNDLLRAIEAQQHLLQLTVWGIKQLQARSGGRGGWMEWDREINNYTSLIHSLIEESQNQQEK

SEQ ID NO: 205 - protein, 63 aa, synthetic construct
  REGION 1..63: note = Synthetic: 2Q3I
  VARIANT 1: note = Xaa can be any naturally occurring amino acid
  VARIANT 47: note = Xaa can be any naturally occurring amino acid
XRMKQIEDKIEEIESKQKKIENEIARIKKLLQLTVWGIKQLQARILXGACGLGQEEWFWLCAA

SEQ ID NO: 206 - protein, 46 aa, synthetic construct
  REGION 1..46: note = Synthetic: 2Q5U
  VARIANT 1: note = Xaa can be any naturally occurring amino acid
XRMKQIEDKIEEIESKQKKIENEIARIKKLLQLTVWGIKQLQARIL

SEQ ID NO: 207 - protein, 46 aa, synthetic construct; identical to SEQ ID NO: 206
  REGION 1..46: note = Synthetic: 2Q7C
  VARIANT 1: note = Xaa can be any naturally occurring amino acid

SEQ ID NO: 208 - protein, 81 aa, synthetic construct
  REGION 1..81: note = Synthetic: 1WT6
GAMAEAEAEVTLRELQEALEEEVLTRQSLSREMEAIRTDNQNFASQLREAEARNRDLEAHVRQLQERMELLQAEGATAVTG

SEQ ID NO: 209 - protein, 55 aa, synthetic construct
  REGION 1..55: note = Synthetic: 2FXP
GSHTSPDVDLGDISGINASVVNIQKEIDRLNEVAKNLNESLIDLQELGKYEQYIK

SEQ ID NO: 210 - protein, 34 aa, synthetic construct
  REGION 1..34: note = Synthetic: 1ZVB
ALNTLVKQLSSNFGAISSVLNDILSRLDKVEAEV

SEQ ID NO: 211 - protein, 86 aa, synthetic construct
  REGION 1..86: note = Synthetic: 1ZV8
NQKQIANQFNKAISQIQESLTTTSTALGKLQDVVNQNAQALNTLVKQLSSDISGINASVVNIQKEIDRLNEVAKNLNESLIDLQEL

SEQ ID NO: 212 - protein, 18 aa, synthetic construct
  REGION 1..18: note = Synthetic: 1S9Z
  VARIANT 1: note = Xaa can be any naturally occurring amino acid
XSIRELEARIRELELRIG

SEQ ID NO: 213 - protein, 278 aa, synthetic construct
  REGION 1..278: note = Synthetic: 1SLQ
AQANEDIVVSKTSLWKEMQYNRDITIRFKFASSIVKSGGLGYKWSEISFKPANYQYTYTRDGEEVTAHTTCSVNGMNDFNFNGGSLPTDFVISRYEVIKENSYVYVDYWDDSQAFRNMVYVRSLAANLNSVICTGGDYSFALPVGQWPVMTGGAVSLHSAGVTLSTQFTDFVSLNSLRFRFRLTVEEPSFSITRTRVSRLYGLPAANPNNGKEYYEVAGRFSLISLVPSNDDYQTPITNSVTVRQDLERQLGELREEFNALSQEIAMSQLIDLALLPL

SEQ ID NO: 214 - protein, 130 aa, synthetic construct
  REGION 1..130: note = Synthetic: 2IBL
MGHHHHHHGSGTDIVLNDLPFVDGPPAEGQSRISWIKNGEEILGADTQYGSEGSMNRPTVSVLRNVEVLDKNIGILKTSLETANSDIKTIQEAGYIPEAPRDGQAYVRKDGEWVLLSTFLGSSGSGLVPR

SEQ ID NO: 215 - protein, 32 aa, synthetic construct
  REGION 1..32: note = Synthetic: 2AKF
VSRLEEDVRNLNAIVQKLQERLDRLEETVQAK

SEQ ID NO: 216 - protein, 35 aa, synthetic construct
  REGION 1..35: note = Synthetic: 2O7H
GSRMKQLEDKVEELLSKNYHLENRVARLEKLVGER

SEQ ID NO: 217 - protein, 75 aa, synthetic construct
  REGION 1..75: note = Synthetic: 2N64
GHMEGKPKMEPAASSQAAVEELRTQVRELRSIIETMKDQQKREIKQLLSELDEEKKIRLRLQMEVNDIKKALQSK

SEQ ID NO: 218 - protein, 72 aa, synthetic construct
  REGION 1..72: note = Synthetic: 2KP8
TSLIHSLIEESQNQQEKNEQELLELDGDGPQLLSGIVQQQNNLLRAIEAQQHLLQLTVWGIKQLQARILAVE

SEQ ID NO: 219 - protein, 74 aa, synthetic construct
  REGION 1..74: note = Synthetic: 1AVY
VEESGLTNKIKAIETDIASVRQEVNTAKGNISSLQGDVQALQEAGYIPEAPRDGQAYVRKDGEWVLLSTFLSPA

SEQ ID NO: 220 - protein, 31 aa, synthetic construct; identical to SEQ ID NO: 192
  REGION 1..31: note = Synthetic: 2JGO
  VARIANT 1: note = Xaa can be any naturally occurring amino acid
  VARIANT 31: note = Xaa can be any naturally occurring amino acid

SEQ ID NO: 221 - protein, 211 aa, synthetic construct; identical to SEQ ID NO: 153
  REGION 1..211: note = Synthetic: 2JJL

SEQ ID NO: 222 - protein, 80 aa, synthetic construct
  REGION 1..80: note = Synthetic: 4I2L
  VARIANT 42..43: note = Xaa can be any naturally occurring amino acid
QQQNNLLRAIEAQQHLLQLTVWGIKQLQARILAVERYLKDQXXMTWETWEREIENYTKQIYKILEESQEQQDRNEKDLLE

SEQ ID NO: 223 - protein, 27 aa, synthetic construct
  REGION 1..27: note = Synthetic: 4J4A
GSLELIKLRIMEIEARIAKIEKDRAIL

SEQ ID NO: 224 - protein, 31 aa, synthetic construct
  REGION 1..31: note = Synthetic: 5K92
  VARIANT 1: note = Xaa can be any naturally occurring amino acid
  VARIANT 31: note = Xaa can be any naturally occurring amino acid
XEWEALEKKLAALESKCQALEKKLEALEHGX

SEQ ID NO: 225 - protein, 36 aa, synthetic construct
  REGION 1..36: note = Synthetic: 5KB1
EWEALEKKLAALESKCQALEKKLQALEKKHEALEHG

SEQ ID NO: 226 - protein, 36 aa, synthetic construct; identical to SEQ ID NO: 225
  REGION 1..36: note = Synthetic: 5KB0

SEQ ID NO: 227 - protein, 36 aa, synthetic construct
  REGION 1..36: note = Synthetic: 5KB2
EWEALEKKLAAAESKCQALEKKLQALEKKLEALEHG

SEQ ID NO: 228 - protein, 78 aa, synthetic construct
  REGION 1..78: note = Synthetic: 4YV3
VDQLTNDKARVEVERDNLAEDIMRLREKLQEEMLQREEAENTLQSFRQDVDNASLARLDLERKVESLQEEIAFLKKLH

SEQ ID NO: 229 - protein, 66 aa, synthetic construct
  REGION 1..66: note = Synthetic: 5ABS
GHMEPAASSQAAVEELRTQVRELRSIIETMKDQQKREIKQLLSELDEEKKIRLRLQMEVNDIKKAL

SEQ ID NO: 230 - protein, 95 aa, synthetic construct
  REGION 1..95: note = Synthetic: 5APY
MKHHHHHHPMSDYDIPTTENLYFQGHMKQLEDKVEELLSKVYHLENEVARLKKLMATKDDIANMKQLEDKVEELLSKVYHLENEVARLKKLVGER

SEQ ID NO: 231 - protein, 120 aa, synthetic construct
  REGION 1..120: note = Synthetic: 5APZ
MKQLEMKVEELLSKVYHLENEVARLKKLITLMQANMATKDDLARMATKDDIANMATKDDIANMATKDDIAKLDVKIENLNTKMKQLEWKVEELLSKVYHLENEVARLKKLVGSGHHHHHH

SEQ ID NO: 232 - protein, 92 aa, synthetic construct
  REGION 1..92: note = Synthetic: 5APQ
MKHHHHHHPMSDYDIPTTENLYFQGHMKQLEDKVEELLSKVYHLENEVARLKKLIENKADMKQLEDKVEELLSKVYHLENEVARLKKLVGER

SEQ ID NO: 233 - protein, 133 aa, synthetic construct
  REGION 1..133: note = Synthetic: 5APP
MKQIEDKIEEILSKIYHIENEIARIKKLIQNVDVRSTENAARSRANEQKIAENKKAIENKADKADVEKNRADIAANSRAIATFRSSSQNIAALTTKMKQIEDKIEEILSKIYHIENEIARIKKLIKLHHHHHH

SEQ ID NO: 234 - protein, 93 aa, synthetic construct
  REGION 1..93: note = Synthetic: 5APS
MKHHHHHHPMSDYDIPTTENLYFQGHMKQLEDKVEELLSKVYHLENEVARLKKLIENKKADMKQLEDKVEELLSKVYHLENEVARLKKLVGER

SEQ ID NO: 235 - protein, 50 aa, synthetic construct; identical to SEQ ID NO: 108
  REGION 1..50: note = Synthetic: 5D5Z

SEQ ID NO: 236 - protein, 31 aa, synthetic construct
  REGION 1..31: note = Synthetic: 5EFM
TDTLLDQLDTQLNVTENECQNYKRCLEILEQ

SEQ ID NO: 237 - protein, 72 aa, synthetic construct
  REGION 1..72: note = Synthetic: 5TOI
GDIIWDQLIVKRTLADLLIPINRQISDIQSTLSEVTTRVHEIERQLHEITPVLKMGRTLEAISKGMSEMLAK

SEQ ID NO: 238 - protein, 72 aa, synthetic construct; identical to SEQ ID NO: 237
  REGION 1..72: note = Synthetic: 5TOH

SEQ ID NO: 239 - protein, 36 aa, synthetic construct
  REGION 1..36: note = Synthetic: 5KKV
  VARIANT 1: note = Xaa can be any naturally occurring amino acid
XGMKQIEDKIEEILSKIYHIENEIARIKKLIGEGHH

SEQ ID NO: 240 - protein, 141 aa, synthetic construct
  REGION 1..141: note = Synthetic: 5IEA
EEGQKVDHCARHGEKLLLFCQEDSKVICWLCERSQEHRGHHTFLMEEVAQEYHVKLQTALEMLRQKQQEAETERNQVAKRVPKAPPEEKEALIARGKALGEQTQYMRELISELEHRLQGSMMDLLQGVDGIIKRIENMTLK

SEQ ID NO: 241 - protein, 198 aa, synthetic construct
  REGION 1..198: note = Synthetic: 4K8U
MCALVSRQRQELQELRRELEELSVGSDGVLIWKIGSYGRRLQEAKAKPNLECFSPAFYTHKYGYKLQVSAFLNGNGSGEGTHLSLYIRVLPGAFDNLLEWPFARRVTFSLLDQSDPGLAKPQHVTETFHPDPNWKNFQKPGTWRGSLDESSLGFGYPKFISHQDIRKRNYVRDDAVFIRAAVELPRKILSLEHHHHHH

SEQ ID NO: 242 - protein, 45 aa, synthetic construct
  REGION 1..45: note = Synthetic: 4GIF
GGWVSGEEFYMLTRRVLQLETVLEGVVSQIDAVGSKLKMLERKGW

SEQ ID NO: 243 - protein, 113 aa, synthetic construct
  REGION 1..113: note = Synthetic: 1AA0
VSGLNNAVQNLQVEIGNNSAGIKGQVVALNTLVNGTNPNGSTVEERGLTNSIKANETNIASVTQEVNTAKGNISSLQGDVQALQEAGYIPEAPRDGQAYVRKDGEWVLLSTFL

SEQ ID NO: 244 - protein, 158 aa, synthetic construct
  REGION 1..158: note = Synthetic: 1B08
EAEAGSVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 245 - protein, 47 aa, synthetic construct
  REGION 1..47: note = Synthetic: 1AQ5
GSHMEEDPCECKSIVKFQTKVEELINTLQQKLEAVAKRIEALENKII

SEQ ID NO: 246 - protein, 155 aa, synthetic construct
  REGION 1..155: note = Synthetic: 1QU1
GTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEEMGNGSFKIYHKCDNACIESIRNGTYDHDVYRDEALNNRFQIKGVELKSGYKDW

SEQ ID NO: 247 - protein, 32 aa, synthetic construct
  REGION 1..32: note = Synthetic: 1PIQ
  VARIANT 1: note = Xaa can be any naturally occurring amino acid
XRMKQIEDKIEEILSKQYHIENEIARIKKLIG

SEQ ID NO: 248 - protein, 177 aa, synthetic construct; identical to SEQ ID NO: 178
  REGION 1..177: note = Synthetic: 1PW9

SEQ ID NO: 249 - protein, 177 aa, synthetic construct; identical to SEQ ID NO: 178
  REGION 1..177: note = Synthetic: 1PWB

SEQ ID NO: 250 - protein, 102 aa, synthetic construct
  REGION 1..102: note = Synthetic: 1SVF
QILSIDPLDISQNLAAVNKSLSDALQHLAQSDTYLSAITAAVALVKANENAAAILNLKNAIQKTNAAVADVVQATQSLGTAVQAVQDHINSVVSPAITAANY

SEQ ID NO: 251 - protein, 33 aa, synthetic construct
  REGION 1..33: note = Synthetic: 1SWI
RMKQLEDKVEELLSKAYHLENEVARLKKLVGER

SEQ ID NO: 252 - protein, 34 aa, synthetic construct
  REGION 1..34: note = Synthetic: 1TGG
  VARIANT 1: note = Xaa can be any naturally occurring amino acid
XAEIEQIKKEIAYLIKKIKEEILEEIKKIKQEIA

SEQ ID NO: 253 - protein, 34 aa, synthetic construct
  REGION 1..34: note = Synthetic: 1ZIM
  VARIANT 1: note = Xaa can be any naturally occurring amino acid
XRMKQLEDKVEELLSKQYHLENEVARLKKLVGER

SEQ ID NO: 254 - protein, 34 aa, synthetic construct
  REGION 1..34: note = Synthetic: 1ZIJ
  VARIANT 1: note = Xaa can be any naturally occurring amino acid
XRMKQLEDKVEELLSKAYHLENEVARLKKLVGER

SEQ ID NO: 255 - protein, 58 aa, synthetic construct
  REGION 1..58: note = Synthetic: 1GZL
  VARIANT 1: note = Xaa can be any naturally occurring amino acid
XRMKQIEDKIEEIESKQKKIENEIARIKKLLQLTVWGIKQLQARILWEEWDREIENYT

SEQ ID NO: 256 - protein, 15 aa, synthetic construct
  REGION 1..15: note = Synthetic: 1HQJ
DELERRIRELEARIK

SEQ ID NO: 257 - protein, 182 aa, synthetic construct
  REGION 1..182: note = Synthetic: 1HTN
GEPPTQKPKKIVNAKKDVVNTKMFEELKSRLDTLSQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 258 - protein, 141 aa, synthetic construct
  REGION 1..141: note = Synthetic: 1HUP
AASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 259 - protein, 34 aa, synthetic construct
  REGION 1..34: note = Synthetic: 1IJ1
  VARIANT 1: note = Xaa can be any naturally occurring amino acid
XRMKQLEDKVEETLSKVYHLENEVARLKKLVGER

SEQ ID NO: 260 - protein, 34 aa, synthetic construct
  REGION 1..34: note = Synthetic: 1IJ0
  VARIANT 1: note = Xaa can be any naturally occurring amino acid
XRMKQLEDKVEESLSKVYHLENEVARLKKLVGER

SEQ ID NO: 261 - protein, 34 aa, synthetic construct
  REGION 1..34: note = Synthetic: 1IJ3
  VARIANT 1: note = Xaa can be any naturally occurring amino acid
XRMKQLEDKVEELLSKSYHLENEVARLKKLVGER

SEQ ID NO: 262 - protein, 34 aa, synthetic construct
  REGION 1..34: note = Synthetic: 1IJ2
  VARIANT 1: note = Xaa can be any naturally occurring amino acid
XRMKQLEDKVEELLSKTYHLENEVARLKKLVGER

SEQ ID NO: 263 - protein, 16 aa, synthetic construct
  REGION 1..16: note = Synthetic: 1KYC
  VARIANT 16: note = Xaa can be any naturally occurring amino acid
EELRRRIEELERRIRX

SEQ ID NO: 264 - protein, 40 aa, synthetic construct
  REGION 1..40: note = Synthetic: 1M7L
GLPDVASLRQQVEALQGQVQHLQAAFSQYKKVELFPNGGI

SEQ ID NO: 265 - protein, 37 aa, synthetic construct
  REGION 1..37: note = Synthetic: 1CE0
RLLQRIKQQEDKLEETLSKIYHLENEIARVKKLLGER

SEQ ID NO: 266 - protein, 31 aa, synthetic construct
  REGION 1..31: note = Synthetic: 1COI
  VARIANT 1: note = Xaa can be any naturally occurring amino acid
  VARIANT 31: note = Xaa can be any naturally occurring amino acid
XEVEALEKKVAALESKVQALEKKVEALEHGX

SEQ ID NO: 267 - protein, 213 aa, synthetic construct
  REGION 1..213: note = Synthetic: 1CUN
MVHQFFRDMDDEESWIKEKKLLVSSEDYGRDLTGVQNLRKKHKRLEAELAAHEPAIQSVLDTGKKLSDDNTIGKEEIQQRLAQFVDHWKELKQLAAARGQRLEESLEYQQFVANVEEEEAWINEKMTLVASEDYGDTLAAIQGLLKKHEAFETDFTVHKDRVNDVCANGEDLIKKNNHHVENITAKMKGLKGKVSDLEKAAAQRKAKLDENSA

SEQ ID NO: 268 - protein, 63 aa, synthetic construct
  REGION 1..63: note = Synthetic: 1CZQ
  VARIANT 1: note = Xaa can be any naturally occurring amino acid
  VARIANT 47: note = Xaa can be any naturally occurring amino acid
XRMKQIEDKIEEIESKQKKIENEIARIKKLLQLTVWGIKQLQARILXGACEARHREWAWLCAA

SEQ ID NO: 269 - protein, 56 aa, synthetic construct
  REGION 1..56: note = Synthetic: 1KFN
SSNAKIDQLSSDVQTLNAKVDQASNDANAARSDAQAAKDDAARANQRLDNMATKYR

SEQ ID NO: 270 - protein, 56 aa, synthetic construct
  REGION 1..56: note = Synthetic: 1KFM
SSNAKIDQLSSDVQTLNAKVDQLSNDVNAARSDAQAAKDDAARANQRLDNMATKYR

SEQ ID NO: 271 - protein, 34 aa, synthetic construct
  REGION 1..34: note = Synthetic: 1GCM
  VARIANT 1: note = Xaa can be any naturally occurring amino acid
XRMKQIEDKIEEILSKIYHIENEIARIKKLIGER

SEQ ID NO: 272 - protein, 175 aa, synthetic construct
  REGION 1..175: note = Synthetic: 4ABX
GIDPFTQRERARQIDLLAFQVQEISEVSPDPGEEEGLNTELSRLSNLHTIAQAAAGGVELLSDGDLNAAGLIGEAVRALNAGAKYDETVMQLQNELRAALESVQAIAGELRDVAEGSAADPEALDRVEARLSALSKLKNKYGPTLEDVVEFGAQAAEELAGLEEDERDAGSLQAD

SEQ ID NO: 273 - protein, 102 aa, synthetic construct
  REGION 1..102: note = Synthetic: 3STQ
MGSSHHHHHHSQDPLEVLFQGPEASMNLKPQTLMVAIQCVAARTRELDAQLQNDDPQNAAELEQLLVGYDLAADDLKNAYEQALGQYSGLPPYDRLIEEPAS

SEQ ID NO: 274 - protein, 190 aa, synthetic construct
  REGION 1..190: note = Synthetic: 3THF
GIDPFTEEPTNLIKQKMDELIKHLNQKIVSLKREQQTISEECSANDRLGQDLFAKLAEKVRPSEASKFRTHVDAVGNITSLLLSLSERLAQTESSLETRQQERGALESKRDLLYEQMEEAQRLKSDIERRGVSIAGLLAKNLSADMCADYDYFINMKAKLIADARDLAVRIKGSEEQLSSLSDALVQSDC

SEQ ID NO: 275 - protein, 260 aa, synthetic construct
  REGION 1..260: note = Synthetic: 3TNU
GSTAEKNRKDAEEWFFTKTEELNREVATNSELVQSGKSEISELRRTMQNLEIELQSQLSMKASLENSLEETKGRYCMQLAQIQEMIGSVEEQLAQLRCEMEQQNQEYKILLDVKTRLEQEIATYRRLLEGEMANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGE

SEQ ID NO: 276 - protein, 101 aa, synthetic construct
  REGION 1..101: note = Synthetic: 3U1C
AGHMDAIKKKMQMLKLDKENALDRAEQAEADKKAAEERSKQLEDDIVQLEKQLRVTEDSRDQVLEELHKSEDSLLFAEENAAKAESEVASLNRRIQLVEEE

SEQ ID NO: 277 - protein, 410 aa, synthetic construct
  REGION 1..410: note = Synthetic: 3TV7
SFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNR

SEQ ID NO: 278 - protein, 422 aa, synthetic construct
  REGION 1..422: note = Synthetic: 4Q62
SNAIKQHQVKLVLKTSQDIQLFLNALRDSRNHGISSLSELDLSDTRFTNQELSDLVTALNNIPGIKSLRLDSCGLKDSDTVELSKLTHIKKLSLKSNYLKNRPMFNSMLEALYLDYNTELSASYVLFSLSRNAAALKKLSLRNCGVTDANLEYLTRPESRLKSLTHFNLRRNNITHQGVDSFAHLQSLTTIDLSQNTGIGDEGVSRLAPLKQLRTLYLDNCGIGGEGIKAIAKMNLQTVDLSFNPGLKKEWGLDDIRPNHTIRTLLLTFCSLNDNHAKLIVSKFPAATDLNVANNNMTRAGVKTLLSNPIIENLDVSTQSLYAKSTGASGTGMISPRIMMKKKEIKQQEKEKAQDLLDTICNTITLKSINLEHTGITSRMLLSLIPDETDHKRYLKKINGVSCKELKPKLEQQIALRKAEKQ

SEQ ID NO: 279 - protein, 61 aa, synthetic construct
  REGION 1..61: note = Synthetic: 4PXJ
GAMDPEFTKNALNVVKNDLIAKVDQLSGEQEVLRGELEAAKQAKVKLENRIKELEEELKRV

SEQ ID NO: 280 - protein, 522 aa, synthetic construct
  REGION 1..522: note = Synthetic: 3VBB
MVLDLDLFRVDKGGDPALIRETQEKRFKDPGLVDQLVKADSEWRRCRFRADNLNKLKNLCSKTIGEKMKKKEPVGDDESVPENVLSFDDLTADALANLKVSQIKKVRLLIDEAILKCDAERIKLEAERFENLREIGNLLHPSVPISNDEDVDNKVERIWGDCTVRKKYSHVDLVVMVDGFEGEKGAVVAGSRGYFLKGVLVFLEQALIQYALRTLGSRGYIPIYTPFFMRKEVMQEVAQLSQFDEELYKVIGKGSEKSDDNSYDEKYLIATSEQPIAALHRDEWLRPEDLPIKYAGLSTCFRQEVGSHGRDTRGIFRVHQFEKIEQFVYSSPHDNKSWEMFEEMITTAEEFYQSLGIPYHIVNIVSGSLNHAASKKLDLEAWFPGSGAFRELVSCSNCTDYQARRLRIRYGQTKKMMDKVEFVHMLNATMCATTRTICAILENYQTEKGITVPEKLKEFMPPGLQELIPFVKPAPIEQEPSKKQKKQHEGSKKKAAARDVTLENRLQNMEVTDALEHHHHHH

SEQ ID NO: 281 - protein, 115 aa, synthetic construct
  REGION 1..115: note = Synthetic: 3VEM
HSAGVTALVPSLLNNGTEQIAVQPVPQIPFPVFNDPFLHELEKLRRESENSKKTFEEKKSILKAELERKMAEVQAEFRRKFHEVEAEHNTRTTKIEKDKNLVIMNKLLANAFLSK

SEQ ID NO: 282 - protein, 51 aa, synthetic construct
  REGION 1..51: note = Synthetic: 3VMZ
GSERQILRLKQINIQLATKIQHLEFSCSEKEQEIERLNKLLKQNGLLGDVN

SEQ ID NO: 283 - protein, 48 aa, synthetic construct
  REGION 1..48: note = Synthetic: 3VMX
GSERQILRLKQINIQLATKIQHLEFSCSEKEQEIERLNKLLKQNGLLG

SEQ ID NO: 284 - protein, 51 aa, synthetic construct; identical to SEQ ID NO: 282
  REGION 1..51: note = Synthetic: 3VMY

SEQ ID NO: 285 - protein, 51 aa, synthetic construct
  REGION 1..51: note = Synthetic: 3VN0
GSERQILRLKQINIQLATKIQHLEFSSSEKEQEIERLNKLLKQNGLLGDVN

SEQ ID NO: 286 - protein, 419 aa, synthetic construct
  REGION 1..419: note = Synthetic: 3W03
SGMEELEQGLLMQPWAWLQLAENSLLAKVFITKQGYALLVSDLQQVWHEQVDTSVVSQRAKELNKRLTAPPAAFLCHLDNLLRPLLKDAAHPSEATFSCDCVADALILRVRSELSGLPFYWNFHCMLASPSLVSQHLIRPLMGMSLALQCQVRELATLLHMKDLEIQDYQESGATLIRDRLKTEPFEENSFLEQFMIEKLPEACSIGDGKPFVMNLQDLYMAVTTQEVQVGQKHQMGSSHHHHHHSSGLVPRGSHMERKISRIHLVSEPSITHFLQVSWEKTLESGFVITLTDGHSAWTGTVSESEISQEADDMAMEKGKYVGELRKALLSGAGPADVYTFNFSKESCYFFFEKNLKDVSFRLGSFNLEKVENPAEVIRELICYCLDTIAENQAKNEHLQKENERLLRDWNDVQGRFEK

SEQ ID NO: 287 - protein, 415 aa, synthetic construct
  REGION 1..415: note = Synthetic: 3NCZ
GSLHMSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNR

SEQ ID NO: 288 - protein, 99 aa, synthetic construct
  REGION 1..99: note = Synthetic: 3NI0
ANSVACRDGLRAQAECRNTTHLLQRQLTRTQDSLLQAETQANSCNLTVVTLQESLEKKVSQALEQQARIKELENEVTKLNQELENLRIQKETSSTVQVN

SEQ ID NO: 289 - protein, 72 aa, synthetic construct
  REGION 1..72: note = Synthetic: 3NMD
MRGSHHHHHHGMASIEGRGSLRDLQYALQEKIEELRQRDALIDELELELDQKDELIQMLQNELDKYRSVIRP

SEQ ID NO: 290 - protein, 77 aa, synthetic construct
  REGION 1..77: note = Synthetic: 3WUT
GAMGSFNSSINNIHEMEIQLKDALEKNQQWLVYDQQREVYVKGLLAKIFELEKKTETAAHSLPDLAVGPPSLNYIPP

SEQ ID NO: 291 - protein, 77 aa, synthetic construct
  REGION 1..77: note = Synthetic: 3WUU
GAMGSFNSSINNIHEMEIQLKDALEKNQQWLVYDQQREVYVKGLLAKIFELEKKTETAAHSLPDLAVGPPSLNYPGY

SEQ ID NO: 292 - protein, 77 aa, synthetic construct
  REGION 1..77: note = Synthetic: 3WUV
GAMGSFNSSINNIHEMEIQLKDALEKNQQWLVYDQQREVYVKGLLAKIFELEKKTETAAHSLPDQAQGPPYPTYIPP

SEQ ID NO: 293 - protein, 260 aa, synthetic construct
  REGION 1..260: note = Synthetic: 4BTA
MHHHHHHHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSPPGPPGPPG

SEQ ID NO: 294 - protein, 239 aa, synthetic construct
  REGION 1..239: note = Synthetic: 4BT8
MHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKE

SEQ ID NO: 295 - protein, 248 aa, synthetic construct
  REGION 1..248: note = Synthetic: 4BT9
MHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEPPGPPGPPG

SEQ ID NO: 296 - protein, 429 aa, synthetic construct
  REGION 1..429: note = Synthetic: 4BWX
GPHMLEIPKDRRENLQKKLFHMQQLLPNSGLPNLDRWHSLFPLDTKATRNSTCFGYPSWMYKAQNNKNGRHFALRRIEGYRLTNEKAILNVTKEWKKIINANIVTVHEAFTTEFFGDSSLIFVYDFHPLSETLYDHHFPPNNSHNRLRNTNKIPENLLWSYVCQIANALLAIHNAKLAARCLELSKIIWENNRIRLAACSILDVLHHDSPNRKTIEELQQEDFVKFGRIILALATNTPTLNFNNIDAALATIVPRYSTQLRGVLEWLIKPSAPGETKTVETLMPMIGARFANFANFVMQESDEKEFHLMRELENGRIARLMFKLSVVNERGDSCGVHNWSETGERLLLKLFRDYVFHQVDADGKARLDTNHYLNCLSKLDASSEEQILLTSRDNATVFVVSYRSIRQMLDRAYGELGKESKPSATGATI

SEQ ID NO: 297 - protein, 429 aa, synthetic construct
  REGION 1..429: note = Synthetic: 4BWK
GPHMLEIPKDRRENLQKKLFHMQQLLPNSGLPNLDRWHSLFPLDTKATRNSTCFGYPSWMYKAQNNKNGRHFALRRIEGYRLTNEKAILNVTKEWKKIINANIVTVHEAFTTEFFGDSSLIFVYDFHPLSETLYDHHFPPNNSHNRLRNTNKIPENLLWSYVCQIANALLAIHNAKLAARCLELSKIIWENNRIRLAACSILDVLHHDSPNRKTIEELQQEDFVKFGRIILALATNTPTLNFNNIDAALATIVPRYSTQLRGVLEWLIKPSAPGETKTVETLLGGITTHLANFANFVMQESDEKEFHLMRELENGRIARLMFKLSVVNERGDSCGVHNWSETGERLLLKLFRDYVFHQVDADGKARLDTNHYLNCLSKLDASSEEQILLTSRDNATVFVVSYRSIRQMLDRAYGELGKESKPSATGATI

SEQ ID NO: 298 - protein, 448 aa, synthetic construct
  REGION 1..448: note = Synthetic: 4BWP
GPHMLEMPDEMRAEVLARNEISNLIMDAAEAAQHALPLEVENYHALYPLEPPAQPLHAKLTFPATTYRATHNTTGYKYCLRRIHGFRLQSTKCMTLVEMWKKLQHTNVVQLREVFTTKAFGDNSLVLVYDYHPGSQTLLAKYFTPAPETNGYTDPFQGEARPFSHKSNMQRTSNGPLLPEATIWSIIMQLTAGLKAIHHAGLACKVLDPTKIIVTGKRVRFSSCCISDITQFDPNASNPLALVNMHQQDDLTALGRLVLALACRCLQSVQRDNVQSSIDMVTRNYSTDLRNFIVYLFTTNNRRSVTDLMPMIGARFYTQLDALQSKIDMQEDELAKEMENGRLYRILVKLNSINERPDFNLDCTWSETGDRYMLKLFRDYLFHSVTEDGRPWLDHAHIVQCLNKLDAGSIERVQLMSRDEQSVLIVSYAELKNCLENAFSELMSSAAN

SEQ ID NO: 299 - protein, 377 aa, synthetic construct
  REGION 1..377: note = Synthetic: 4CG4
  VARIANT 1: note = Xaa can be any naturally occurring amino acid
XSEVALEHKKKIQKQLEHLKKLRKSGEEQRSYGEEKAVSFLKQTEALKQRVQRKLEQVYYFLEQQEHFFVASLEDVGQMVGQIRKAYDTRVSQDIALLDALIGELEAKECQSEWELLQDIGDILHRAKTVPVPEKWTTPQEIKQKIQLLHQKSEFVEKSTKYFSETLRSEMEMFNVPELIGAQAHAVNVILDAETAYPNLIFSDDLKSVRLGNKWERLPDGPQRFDSCIIVLGSPSFLSGRRYWEVEVGDKTAWILGACKTSISRKGNMTLSPENGYWVVIMMKENEYQASSVPPTRLLIKEPPKRVGIFVDYRVGSISFYNVTARSHIYTFASCSFSGPLQPIFSPGTRDGGKNTAPLTICPVGGQGPDALEVLFQ

SEQ ID NO: 300 - protein, 630 aa, synthetic construct
  REGION 1..630: note = Synthetic: 4CFG
MAELCPLAEELSCSICLEPFKEPVTTPCGHNFCGSCLNETWAVQGSPYLCPQCRAVYQARPQLHKNTVLCNVVEQFLQADLAREPPADVWTPPARASAPSPNAQVACDHCLKEAAVKTCLVCMASFCQEHLQPHFDSPAFQDHPLQPPVRDLLRRKCSQHNRLREFFCPEHSECICHICLVEHKTCSPASLSQASADLEATLRHKLTVMYSQINGASRALDDVRNRQQDVRMTANRKVEQLQQEYTEMKALLDASETTSTRKIKEEEKRVNSQFDTIYQILLKKKSEIQTLKEEIEQSLTKRDEFEFLEKASKLRGISTQPVYIPEVELNHKLIKGIHQSTIDLKNELKQCIGRLQEPTPSSGDPGEHDPASTHKSTRPVKKVSKEEKKSKKPPPVPALPSKLPTFGAPEQLVDLKQAGLEAAAKATSSHPNSTSLKAKVLETFLAKSRPELLEYYIKVILDYNTAHNKVALSECYTVASVAEMPQNYRPHPQRFTYCSQVLGLHCYKKGIHYWEVELQKNNFCGVGICYGSMNRQGPESRLGRNSASWCVEWFNTKISAWHNNVEKTLPSTKATRVGVLLNCDHGFVIFFAVADKVHLMYKFRVDFTEALYPAFWVFSAGATLSICSPK

SEQ ID NO: 301 - protein, 180 aa, synthetic construct
  REGION 1..180: note = Synthetic: 4CKM
GPHMEAVESGVAPPPAVRTARTDLPALASPKHSAEEPQTLLETTVMVSTKMPPHEPQVRPLGVYVRTGRGGPNGVTRVVLVRLTDPTDPFFLFELELLEDDYNAFKQHLELLVDFHGFPRYLVGMLRDIADGASAYELSFVLNSAAVGDSNRGTLRVLETTDFKTVEHISLVLLRQGDAG

SEQ ID NO: 302 - protein, 226 aa, synthetic construct
  REGION 1..226: note = Synthetic: 4CKN
GPHMEAVESGVAPPPAVRTARTDLPALASPKHSAEEPQTLLETTVMVSTKMPPHEPQVRPLGVYVRTGRGGPNGVTRVVLVRLTDPTDPFFLFELELLEDDYNAFKQHLELLVDFHGFPRYLVGMLRDIADGASAYELSFVLNSAAVGDSNRGTLRVLETTDEKTVEHISLVLLRQGDAGLKRYLAERFQHYEQSFRASEASRAVITAELQEKIGDLQAANDALRA

SEQ ID NO: 303 - protein, 382 aa, synthetic construct
  REGION 1..382: note = Synthetic: 4CKG
GPLGSMTVKLDFEECLKDSPRFRASIELVEAEVSELETRLEKLLKLGTGLLESGRHYLAASRAFVVGICDLARLGPPEPMMAECLEKFTVSLNHKLDSHAELLDATQHTLQQQIQTLVKEGLRGFREARRDFWRGAESLEAALTHNAEVPRRRAQEAEEAGAALRTARAGYRGRALDYALQINVIEDKRKFDIMEFVLRLVEAQATHFQQGHEELSRLSQYRKELGAQLHQLVLNSAREKRDMEQRHVLLKQKELGGEEPEPSLREGPGGLVMEGHLFKRASNAFKTWSRRWFTIQSNQLVYQKKYKDPVTVVVDDLRLCTVKLCPDSERRFCFEVVSTSKSCLLQADSERLLQLWVSAVQSSIASAFSQARLDDSPRGPGQ

SEQ ID NO: 304 - protein, 382 aa, synthetic construct; identical to SEQ ID NO: 303
  REGION 1..382: note = Synthetic: 4CKH

SEQ ID NO: 305 - protein, 382 aa, synthetic construct; identical to SEQ ID NO: 303
  REGION 1..382: note = Synthetic: 4NSW

SEQ ID NO: 306 - protein, 51 aa, synthetic construct
  REGION 1..51: note = Synthetic: 4OH9
GSDFDFLKNLSLEELQMRLKALDPMMEREIEELRQRYTAKRQPILDAMDAK

SEQ ID NO: 307 - protein, 246 aa, synthetic construct
  REGION 1..246: note = Synthetic: 4OJK
GSYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNNLSFIETSALDSTNVEEAFKNILTEIYRIVSQKQIADRAAHDESPGNNVVDISVPPTTDGSKDAELQEREYHLKELREQLAKQTVAIAELTEELQSKCIQLNKLQ

SEQ ID NO: 308 - protein, 228 aa, synthetic construct
  REGION 1..228: note = Synthetic: 3Q0X
GSMPLLLDDGDPKAQTGFDLSTATTLFWRPVPVHVKQQDREDVLEELTFRILTGVAKQNHNLRILRIHISSDSDLFFLHTLEVSEEDFQSLKNDQGILVDFASFPGKIISLLEKCILAQPGDSPRFQAVLTIRGGESVFKIVEINDEKQLPHITLAFRPGNDSVVKQFLAFRLSEVKGTCHDLSDDLSRTRDDRDSMVAQLAQCRQQLAQLREQYDKHLLEVQAQAKT

SEQ ID NO: 309 - protein, 96 aa, synthetic construct
  REGION 1..96: note = Synthetic: 3Q8T
GPGSDSEQLQRELKELALEEERLIQELEDVEKNRKVVAENLEKVQAEAERLDQEEAQYQREYSEFKRQQLELDDELKSVENQMRYAQMQLDKLKKK

SEQ ID NO: 310 - protein, 339 aa, synthetic construct
  REGION 1..339: note = Synthetic: 3QKT
MKLERVTVKNFRSHSDTVVEFKEGINLIIGQNGSGKSSLLDAILVGLYWPLRIKDIKKDEFTKVGARDTYIDLIFEKDGTKYRITRRFLKGYSSGEIHAMKRLVGNEWKHVTEPSSKAISAFMEKLIPYNIFLNAIYIRQGQIDAILESDEAREKVVREVLNLDKFETAYKKLSELKGGSGGTEELIEKVKKYKALAREAALSKIGELASEIFAEFTEGKYSEVVVRAEENKVRLFVVWEGKERPLTFLSGGERIALGLAFRLAMSLYLAGEISLLILDEPTPYLDEERRRKLITIMERYLKKIPQVILVSHDEELKDAADHVIRISLENGSSKVEVVS

SEQ ID NO: 311 - protein, 115 aa, synthetic construct
  REGION 1..115: note = Synthetic: 3QFL
AAISNLIPKLGELLTEEFKLHKGVKKNIEDLGKELESMNAALIKIGEVPREQLDSQDKLWADEVRELSYVIEDVVDKFLVQVDGIKSDDNNNKFKGLMKRTTELLKKVKHKHGIA

SEQ ID NO: 312 - protein, 81 aa, synthetic construct
  REGION 1..81: note = Synthetic: 3QH9
KLKLVGMEKEQREQEEKQRKAEELLQELRHLKIKVEELENERNQYEWKLKATKAEVAQLQEQVALKDAEIERLHSQLSRTA

SEQ ID NO: 313 - protein, 109 aa, synthetic construct
  REGION 1..109: note = Synthetic: 4ETO
MACPLEKALDVMVSTFHKYSGKEGDKFKLNKSELKELLTRELPSFLGKRTDEAAFQKLMSNLDSNRDNEVDFQEYCVFLSCIAMMCNEFFEGFDAMNREVSSLKNKLRR

SEQ ID NO: 314 - protein, 823 aa, synthetic construct
  REGION 1..823: note = Synthetic: 4G0U
MASWSHPQFEKGADDDDKVPDPTSVDSVKYSKIKGIPKLDDANDAGGKHSLECTLILTEGDSAKSLAVSGLGVIGRDRYGVFPLRGKILNVREASHKQIMENAEINNIIKIVGLQYKKSYDDAESLKTLRYGKIMIMTDQDQDGSHIKGLLINFIHHNWPSLLKHGFLEEFITPIVKASKNKQELSFYSIPEFDEWKKHIENQKAWKIKYYKGLGTSTAKEAKEYFADMERHRILFRYAGPEDDAAITLAFSKKKIDDRKEWLTNFMEDRRQRRLHGLPEQFLYGTATKHLTYNDFINKELILFSNSDNERSIPSLVDGFKPGQRKVLFTCFKRNDKREVKVAQLAGSVAEMSAYHHGEQALMMTIVNLAQNFVGSNNINLLQPIGQFGTRLHGGKDAASPRYIFTMLSTLARLLFPAVDDNLLKFLYDDNQRVEPEWYIPIIPMVLINGAEGIGTGWACKLPNYDAREIVNNVRRMLDGLDPHPMLPNYKNFKGTIQELGQNQYAVSGEIFVVDRNTVEITELPVRTWTQVYKEQVLEPMLNGTDKTPALISDYKEYHTDTTVKFVVKMTEEKLAQAEAAGLHKVFKLQTTLTCNSMVLFDHMGCLKKYETVQDILKEFFDLRLSYYGLRKEWLVGMLGAESTKLNNQARFILEKIQGKITIENRSKKDLIQMLVQRGYESDPVKAWKEAQEKAAEEDETQNQHDDSSSDSGTPSGPDFNYILNMSLWSLTKEKVEELIKQRDAKGREVNDLKRKSPSDLWKEDLAAFVEELDKVESQEREDGAPGFSSISAHHHHHHHHHHAGCCGAGCTGCAGCTCGGCT

SEQ ID NO: 315 - protein, 823 aa, synthetic construct; identical to SEQ ID NO: 314
  REGION 1..823: note = Synthetic: 4G0V

SEQ ID NO: 316 - protein, 823 aa, synthetic construct; identical to SEQ ID NO: 314
  REGION 1..823: note = Synthetic: 4G0W

SEQ ID NO: 317 - protein, 142 aa, synthetic construct
  REGION 1..142: note = Synthetic: 4G79
GSMTSKIALFDQTLIASLLQPLSLNQPDFKAYKTKVKLKISEQRNETSGEKELKFEISRSDDFEFLFSETLNNEKYQILARDHDLTVDFDAFPKVIIQHLLCKNTEINIILDAEKNFCSFELFSKTPISKGKIFSIKLHAVR

SEQ ID NO: 318 - protein, 141 aa, synthetic construct
  REGION 1..141: note = Synthetic: 3OKQ
GSAKNSSNRMYMEKSQTELGDLSDTLLSKVDDLQDVIEIMRKDVAERRSQPAKKKLETVSKDLENAQADVLKLQEFIDTEKPHWKKTWEAELDKVCEEQQFLTLQEELILDLKEDLGKALETFDLIKLCCEEQEKNPSRSK

SEQ ID NO: 319 - protein, 141 aa, synthetic construct; identical to SEQ ID NO: 318
  REGION 1..141: note = Synthetic: 3ONX

SEQ ID NO: 320 - protein, 313 aa, synthetic construct
  REGION 1..313: note = Synthetic: 3CL3
GAMGSMATYEVLCEVARKLGTDDREVVLFLLNVFIPQPTLAQLIGALRALKEEGRLTFPLLAECLFRAGRRDLLRDLLHLDPRFLERHLAGTMSYFSPYQLTVLHVDGELCARDIRSLIFLSKDTIGSRSTPQTFLHWVYCMENLDLLGPTDVDALMSMLRSLSRVDLQRQVQTLMGLHLSGPGHMASGSLGELQESQSRLEAATKECQALEGRARAASEQARQLESEREALQQQHSVQVDQLRMQGQSVEAALRMERQAASEEKRKLAQLQVAYHQLFQEYDNHIKSSVVGSERKRGMQLEDLKQQLQQAEE

SEQ ID NO: 321 - protein, 415 aa, synthetic construct; identical to SEQ ID NO: 287
  REGION 1..415: note = Synthetic: 3D9V

SEQ ID NO: 322 - protein, 71 aa, synthetic construct
  REGION 1..71: note = Synthetic: 3E1R
FNSSINNIHEMEIQLKDALEKNQQWLVYDQQREVYVKGLLAKIFELEKKTETAAHSLPQAQGPPYPTYPGY

SEQ ID NO: 323 - protein, 66 aa, synthetic construct
  REGION 1..66: note = Synthetic: 3HLS
GSHMATRDLVLLGEQFREEYKLTQELEMLTDRLQLTLRALEDEKKKTDTLLYSVLPPSVANELRHK

SEQ ID NO: 324 - protein, 138 aa, synthetic construct
  REGION 1..138: note = Synthetic: 3HNW
SNAMSSKNTAEVILGGKVIKLGGYESEEYLQRVASYINNKITEFNKEESYRRMSAELRTDMMYLNIADDYFKAKKMADSLSLDIENKDKEIYDLKHELIAAQIKAESSAKEIKELKSEINKYQKNIVKLETELNDSKK

SEQ ID NO: 325 - protein, 33 aa, synthetic construct
  REGION 1..33: note = Synthetic: 3I1G
RMAQLEAKVEELLSKNWNLENEVARLKKLVGER

SEQ ID NO: 326 - protein, 214 aa, synthetic construct
  REGION 1..214: note = Synthetic: 2VY2
LGTERQREHPFIVTEPGEVARGKKNGLDYLFHLYEQCREFLLQVQTIAKDRGEKCPTKVTNQVFRYAKKSGASYINKPKMRHYVHCYALHCLDEEASNALRRAFKERGENVGSWRQACYKPLVNIACRHGWDIDAVFNAHPRLSIWYVPTKLRQLCHLERNNAVAAAAALVGGISCTGSSTSGRGGCGGDDLRFATTTAATCCAATGGTTACAA

SEQ ID NO: 327 - protein, 63 aa, synthetic construct
  REGION 1..63: note = Synthetic: 2W6A
GPLGSSDGAVTLQEYLELKKALATSEAKVQQLMKVNSSLSDELRKLQREIHKLQAENLQLRQP

SEQ ID NO: 328 - protein, 162 aa, synthetic construct
  REGION 1..162: note = Synthetic: 2WMM
HMERDEVGAHKNAVDEEIERLSQPGGSEDQRLNALAERFGGVLLSEIYDDVSLEDAPYFSALYGPSRHAIVVPDLSQVTEHLEGLTDCPEDLYLIEGDPQSFDDSVFSVDELEKAVVVKIADRQWRYSRFPEVPLFGRAARESRIESLHAEREVLSERFATL

SEQ ID NO: 329 - protein, 755 aa, synthetic construct
  REGION 1..755: note = Synthetic: 2WVR
MNPSMKQKQEEIKENIKNSSVPRRTLKMIQPSASGSLVGRENELSAGLSKRKHRNDHLTSTTSSPGVIVPESSENKNLGGVTQESFDLMIKENPSSQYWKEVAEKRRKALYEALKENEKLHKEIEQKDNEIARLKKENKELAEVAEHVQYMAELIERLNGEPLDNFESLDNQEFDSEEETVEDSLVEDSEIGTCAEGTVSSSTDAKPCIMEQRRVTDFFARRRPGPPRIAPPKLACRTPSPARPALRAPASATSGSRKRARPPAAPGRDQARPPARRRLRLSVDEVSSPSTPEAPDIPACPSPGQKIKKSTPAAGQPPHLTSAQDQDTISELASCLQRARELGARVRALKASAQDAGESCTPEAEGRPEEPCGEKAPAYQRFHALAQPGLPGLVLPYKYQVLAEMFRSMDTIVGMLHNRSETPTFAKVQRGVQDMMRRRFEERNVGQIKTVYPASYRFRQERSVPTFKDGTRRSDYQLTIEPLLEQEADGAAPQLTASRLLQRRQIFSQKLVEHVKEHHKAFLASLSPAMVVPEDQLTRWHPRFNVDEVPDIEPAALPQPPATEKLTTAQEVLARARNLISPRMEKALSQLALRSAAPSSPGSPRPALPATPPATPPAASPSALKGVSQDLLERIRAKEAQKQLAQMTRCPEQEQRLQRLERLPELARVLRSVFVSERKPALSMEVACARMVGSCCTIMSPGEMEKHLLLLSELLPDWLSLHRIRTDTYVKLDKAADLAHITARLAHQTRAEEGL

SEQ ID NO: 330 - protein, 455 aa, synthetic construct
  REGION 1..455: note = Synthetic: 2ZR2
MLDIKLIRENPELVKNDLIKRGELEKVKWVDEILKLDTEWRTKLKEINRLRHERNKIAVEIGKRRKKGEPVDELLAKSREIVKRIGELENEVEELKKKIDYYLWRLPNITHPSVPVGKDENDNVPIRFWGKARVWKGHLERFLEQSQGKMEYEILEWKPKLHVDLLEILGGADFARAAKVSGSRFYYLLNEIVILDLALIRFALDRLIEKGFTPVIPPYMVRRFVEEGSTSFEDFEDVIYKVEDEDLYLIPTAEHPLAGMHANEILDGKDLPLLYVGVSPCFRKEAGTAGKDTKGIFRVHQFHKVEQFVYSRPEESWEWHEKIIRNAEELFQELEIPYRVVNICTGDLGYVAAKKYDIEAWMPGQGKFREVVSASNCTDWQARRLNIRFRDRTDEKPRYVHTLNSTAIATSRAIVAILENHQEEDGTVRIPKVLWKYTGFKEIVPVEKKERCCAT

SEQ ID NO: 331 - protein, 455 aa, synthetic construct; identical to SEQ ID NO: 330
  REGION 1..455: note = Synthetic: 2ZR3

SEQ ID NO: 332 - protein, 34 aa, synthetic construct
  REGION 1..34: note = Synthetic: 2ZTA
  VARIANT 1: note = Xaa can be any naturally occurring amino acid
XRMKQLEDKVEELLSKNYHLENEVARLKKLVGER

SEQ ID NO: 333 - protein, 177 aa, synthetic construct
  REGION 1..177: note = Synthetic: 3A6M
MEERNHENTLEKDLEAVGQEAQALEERLKAAEEELKGLKDKYLRLLADFDNYRKRMEEELKAREREGVLKALRALLPVLDDLDRALEFAEASPESIRQGVRAIRDGFFRILAGLGVEEVPGEGEAFDPRYHEAVGLLPGEPGKVAKVFQRGFRMGEALVRPARVAVGEEKREEADLE

SEQ ID NO: 334 - protein, 75 aa, synthetic construct
  REGION 1..75: note = Synthetic: 3A7O
GSGAIGGNIVSHDDALLNTLAILQKELKSKEQEIRRLKEVIALKNKNTERLNDELISGTIENNVLQQKLSDLKKE

SEQ ID NO: 335 - protein, 312 aa, synthetic construct
  REGION 1..312: note = Synthetic: 3ABH
GSSGSSGMSVTYDDSVGVEVSSDSFWEVGNYKRTVKRIDDGHRLCSDLMNCLHERARIEKAYAQQLTEWARRWRQLVEKGPQYGTVEKAWMAFMSEAERVSELHLEVKASLMNDDFEKIKNWQKEAFHKQMMGGFKETKEAEDGFRKAQKPWAKKLKEVEAAKKAHHAACKEEKLAISREANSKADPSLNPEQLKKLQDKIEKCKQDVLKTKEKYEKSLKELDQGTPQYMENMEQVFEQCQQFEEKRLRFFREVLLEVQKHLDLSNVAGYKAIYHDLEQSIRAADAVEDLRWFRANHGPGMAMNWPQFEEWS

SEQ ID NO: 336 - protein, 350 aa, synthetic construct
  REGION 1..350: note = Synthetic: 3ACO
GSSGSSGMSVTYDDSVGVEVSSDSFWEVGNYKRTVKRIDDGHRLCSDLMNCLHERARIEKAYAQQLTEWARRWRQLVEKGPQYGTVEKAWMAFMSEAERVSELHLEVKASLMNDDFEKIKNWQKEAFHKQMMGGFKETKEAEDGFRKAQKPWAKKLKEVEAAKKAHHAACKEEKLAISREANSKADPSLNPEQLKKLQDKIEKCKQDVLKTKEKYEKSLKELDQGTPQYMENMEQVFEQCQQFEEKRLRFFREVLLEVQKHLDLSNVAGYKAIYHDLEQSIRAADAVEDLRWFRANHGPGMAMNWPQFEEWSADLNRTLSRREKKKATDGVTLTGINQTGDQSLPSKPSS

SEQ ID NO: 337 - protein, 150 aa, synthetic construct
  REGION 1..150: note = Synthetic: 3AJW
GSHMAQHGALETLKDLAEKEVDDAARLLGEMRRGCQQAEEQLKMLIDYQNEYRSNLNTDMGNGIASNRWINYQQFIQTLEKAIEQHRLQLTQWTQKVDLALKSWREKKQRLQAWQTLQDRQTAAALLAENRMDQKKMDEFAQRAAMRKPE

SEQ ID NO: 338 - protein, 1442 aa, synthetic construct
  REGION 1..1442: note = Synthetic: 3GHG
ADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDVGHRPGPRP

SEQ ID NO: 339 - protein, 39 aa, synthetic construct
  REGION 1..39: note = Synthetic: 3G1E
  VARIANT 1: note = Xaa can be any naturally occurring amino acid
  VARIANT 39: note = Xaa can be any naturally occurring amino acid
XNEKVELQELNDRFANLIDKVRFLEQQNKILLAELEQLX

SEQ ID NO: 340 - protein, 278 aa, synthetic construct
  REGION 1..278: note = Synthetic: 3IAO
MKESYYSIGEVSKLANVSIKALRYYDKIDLFKPAYVDPDTSYRYYTDSQLIHLDLIKSLKYIGTPLEEMKKAQDLEMEELFAFYTEQERQIREKLDFLSALEQTISLVKKRMKRQMEYPALGEVFVLDEEEIRIIQTEAEGIGPENVLNASYSKLKKFIESADGFTNNSYGATFSFQPYTSIDEMTYRHIFTPVLTNKQISSITPDMEITTIPKGRYACIAYNFSPEHYFLNLQKLIKYIADRQLTVVSDVYQLIIPIHYSPKKQEEYRVEMKIEIAE

SEQ ID NO: 341 - protein, 500 aa, synthetic construct
  REGION 1..500: note = Synthetic: 3ID6
MAEVITVKQTNMENIYECEFNDGSFRLCTRNLVPNFNVYGERLIKYEGVEYREWNAFRSKLAGAILKGLKTNPIRKGTKVLYLGAASGTTISHVSDIIELNGKAYGVEFSPRVVRELLLVAQRRPNIFPLLADARFPQSYKSVVENVDVLYVDIAQPDQTDIAIYNAKFFLKVNGDMLLVIKARSIDVTKDPKEIYKTEVEKLENSNFETIQIINLDPYDKDHAIVLSKYKGMVKIYLIEHVIGAVAYDENGNIVDYITNPRDLGKITEELLNNEKGIPFSATVELLKKVNPQEVVVENEAEVPKLQALGYRVSYEPYSKVSRIFRESLPKVAIDIKFASNEEDYYNFLHELSLEYTRRKLRSAAQKRDLLAIQAVRAMDDIDKTINLFSERLREWYSIHFPELDKLIEDHEEYATIVSRFGDRGFLTIDSLKELGFNEQRINRILDAAKKSIGADISEDDLSAMRMIANTILDLYNIRRNLNNYLEGVMKEVAHHHHHH

SEQ ID NO: 342 - protein, 255 aa, synthetic construct
  REGION 1..255: note = Synthetic: 3ICX
MKRDLLAIQAVRAMDDIDKTINLFSERLREWYSIHFPELDKLIEDHEEYATIVSRFGDRGFLTIDSLKELGFNEQRINRILDAAKKSIGADISEDDLSAMRMIANTILDLYNIRRNLNNYLEGVMKEVAPNVTALVGPALGARLLSIAGSLDELAKMPASTIQVLGAEKALFRALRSGGRPPKHGIIFQYPAIHTSPRWQRGKIARALAAKLAIAARVDAFSGRFIGDQLNEQLKKRIDEIKEKFAQHHHHHHHH

SEQ ID NO: 343 - protein, 750 aa, synthetic construct
  REGION 1..750: note = Synthetic: 3ID5
MVKIYLIEHVIGAVAYDENGNIVDYITNPRDLGKITEELLNNEKGIPFSATVELLKKVNPQEVVVENEAEVPKLQALGYRVSYEPYSKVSRIFRESLPKVAIDIKFASNEEDYYNFLHELSLEYTRRKLRSAAQKRDLLAIQAVRAMDDIDKTINLFSERLREWYSIHFPELDKLIEDHEEYATIVSRFGDRGFLTIDSLKELGFNEQRINRILDAAKKSIGADISEDDLSAMRMIANTILDLYNIRRNLNNYLEGVMKEVAPNVTALVGPALGARLLSIAGSLDELAKMPASTIQVLGAEKALFRALRSGGRPPKHGIIFQYPAIHTSPRWQRGKIARALAAKLAIAARVDAFSGRFIGDQLNEQLKKRIDEIKEKFAQHHHHHHHHMAEVITVKQTNMENIYECEFNDGSFRLCTRNLVPNFNVYGERLIKYEGVEYREWNAFRSKLAGAILKGLKTNPIRKGTKVLYLGAASGTTISHVSDIIELNGKAYGVEFSPRVVRELLLVAQRRPNIFPLLADARFPQSYKSVVENVDVLYVDIAQPDQTDIAIYNAKFFLKVNGDMLLVIKARSIDVTKDPKEIYKTEVEKLENSNFETIQIINLDPYDKDHAIVLSKYKGMDAMSKASYVKFEVPQDLADKVLEAVRKAKESGKIKKGTNETTKAVERGQAKLVIIAEDVQPEEIVAHLPLLCDEKKIPYVYVSSKKALGEACGLQVATASAAILEPGEAKDLVDEIIKRVNEIKGKTSS

SEQ ID NO: 344 - protein, 37 aa, synthetic construct
  REGION 1..37: note = Synthetic: 3AZD
AGSSSLEAVRRKIRSLQEQNYHLENEVARLKKLVGER

SEQ ID NO: 345 - protein, 282 aa, synthetic construct
  REGION 1..282: note = Synthetic: 3BBP
GPGMSTGGDFGNPLRKFKLVFLGEQSVGKTSLITRFMYDSFDNTYQATIGIDFLSKTMYLEDRTVRLQLWDTAGLERFRSLIPSYIRDSTVAVVVYDITNVNSFQQTTKWIDDVRTERGSDVIIMLVGNKTDLADKRQVSIEEGERKAKELNVMFIETSAKAGYNVKQLFRRVAAALPGMESTQDRSREDMIDIKLEKPQEQPVSEGGCLLGPLGSLEPPLWHAEFTKEELVQKLSSTTKSADHLNGLLRETEATNAILMEQIKLLKSEIRRLERNQEREKS

SEQ ID NO: 346 - protein, 89 aa, synthetic construct
  REGION 1..89: note = Synthetic: 3BAS
GSHMPLLSIARQEEEMKEQLKQMDKMKEDLAKTERIKKELEEQNVTLLEQKNDLFGSMKQLEDKVEELLSKNYHLENEVARLKKLVGER

SEQ ID NO: 347 - protein, 89 aa, synthetic construct; identical to SEQ ID NO: 346
  REGION 1..89: note = Synthetic: 3BAT

SEQ ID NO: 348 - protein, 276 aa, synthetic construct
  REGION 1..276: note = Synthetic: 2V0O
LGSPMAYFVENFWGEKNSGFDVLYHNMKHGQISTKELADFVRERATIEEAYSRSMTKLAKSASNYSQLGTFAPVWDVFKTSTEKLANCHLDLVRKLQELIKEVQKYGEEQVKSHKKTKEEVAGTLEAVQTIQSITQALQKSKENYNAKCVEQERLKKEGATQREIEKAAVKSKKATDTYKLYVEKYALAKADFEQKMTETAQKFQDIEETHLIHIKEIIGSLSNAIKEIHLQIGQVHEEFINNMANTTVESLIQKFAESKGTGKERPGLIEFEECD

SEQ ID NO: 349 - protein, 409 aa, synthetic construct
  REGION 1..409: note = Synthetic: 2V51
MCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCFLSERKNVLQLKLQQRRTREELVSQGIMPPLKS

SEQ ID NO: 350 - protein, 189 aa, synthetic construct
  REGION 1..189: note = Synthetic: 2V71
GAMDFSSLKEETAYWKELSMKYKQSFQEARDELVEFQEGSRELEAELEAQLVQAEQRNRDLQADNQRLKYEVEALKEKLEHQYAQSYKQVSVLEDDLSQTRAIKEQLHKYVRELEQANDDLERAKRATIMSLEDFEQRLNQAIERNAFLESELDEKESLLVSVQRLKDEARDLRQELAVRERQQEVTRK

SEQ ID NO: 351 - protein, 182 aa, synthetic construct
  REGION 1..182: note = Synthetic: 2Y3W
GPHMTELLFNKRLQVLVKSKDTDERRSVIRVSIELQLPSSPVHRKDLVVRLTDDTDLYFLYNLIISEEDFQSLKVQQGLLIDFTSFPQKFIDLLEQCICEQDKENPRFLLQLSSSSSAFDHSPSNLNIVETNADKHLTHLSLKLLPGSDTDIKKYLASCLSSVKEEKQQLQQKLRKTEEDLT

SEQ ID NO: 352 - protein, 72 aa, synthetic construct
  REGION 1..72: note = Synthetic: 2XU6
GPQTLVNSLEFLNIQKNSTMSEIRDIEVEVENLRQKKEKLLGKIANIEQNQLMLEDNLKQIDDRLDFLEEYG

SEQ ID NO: 353 - protein, 74 aa, synthetic construct
  REGION 1..74: note = Synthetic: 2XV5
GGSARERDTSRRLLAEKEREMAEMRARMQQQLDEYQELLDIKLALDMEIHAYRKLLEGEEERLRLSPSPTSQRS

SEQ ID NO: 354 - protein, 411 aa, synthetic construct
  REGION 1..411: note = Synthetic: 4W7P
GASTGDSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSAN

SEQ ID NO: 355 - protein, 730 aa, synthetic construct
  REGION 1..730: note = Synthetic: 4WID
GPLGSKQARKDMALQHAVDLLEKMLADEEKKLTEFNLGDPLFESANDDPIKTLEEIIQEGDDVVGAHQLVVTQIKLRVQRNRRLADEIIREQLTDIRKVFSDKFEKLEQGIQNSYLLLDKLKTPFQDMRCLFEVANEQFNDTPVPPQYKEKFMVCLKQIVQYAVNSSSKLEKFVMLKIKTKKDDIKDRVTYTCMKYLLMAMQGTGGPKAINNEEHAKLFFKQLSNYDDLTDANHDGLELIKKLDKEQKEVAFHVNNFTHLVTTLGMALYKEGHQKNDEAMLGMHTPITMLSDQVRVLILYLIDEIVHAIHTNSNQSNDELIDGLKPKVRIVINEFHATLMMGIDKMKFYSLNELREIVNDKINEDGPLGSKQARKDMALQHAVDLLEKMLADEEKKLTEFNLGDPLFESANDDPIKTLEEIIQEGDDVVGAHQLVVTQIKLRVQRNRRLADEIIREQLTDIRKVFSDKFEKLEQGIQNSYLLLDKLKTPFQDMRCLFEVANEQFNDTPVPPQYKEKFMVCLKQIVQYAVNSSSKLEKFVMLKIKTKKDDIKDRVTYTCMKYLLMAMQGTGGPKAINNEEHAKLFFKQLSNYDDLTDANHDGLELIKKLDKEQKEVAFHVNNFTHLVTTLGMALYKEGHQKNDEAMLGMHTPITMLSDQVRVLILYLIDEIVHAIHTNSNQSNDELIDGLKPKVRIVINEFHATLMMGIDKMKFYSLNELREIVNDKINED

SEQ ID NO: 356 - protein, 392 aa, synthetic construct
  REGION 1..392: note = Synthetic: 4WHV
GPLGSPEFMAGLPRRIIKETQRLLAEPVPGIKAEPDESNARYFHVVIAGPQDSPFEGGTFKLELFLPEEYPMAAPKVRFMTKIYHPNVDKLGRIKLDILKDKWSPALQIRTVLLSIQALLSAPNPDDPLANDVAEQWKTNEAQAIETARAWTRLYAMNNIGPLGSPEFQEHWALMEELNRSKKDFEAIIQAKNKELEQTKEEKEKMQAQKEEVLSHMNDVLENELQCIICSEYFIEAVTLNCAHSFCSYCINEWMKRKIECPICRKDIKSKTYSLVLDNCINKMVNNLSSEVKERRIVLIRERKAKRLFGPGYQDPMQIFVKTLTGKTITLEVEPSDTIENVKAKIQDKEGIPPDQQRLIFAGKQLEDGRTLSDYNIQKESTLHLVLRLRGG

SEQ ID NO: 357 - protein, 263 aa, synthetic construct
  REGION 1..263: note = Synthetic: 4WII
GAMEGFKANLSLLRRPGEKTYTQRCRLFVGNLPADITEDEFKRLFAKYGEPGEVFINKGKGFGFIKLESRALAEIAKAELDDTPMRGRQLRVRFATHAAALSVRNLSPYVSNELLEEAFSQFGPIERAVVIVDDRGRSTGKGIVEFASKPAARKAFERCSEGVFLLTTTPRPVIVEPLEQLDDEDGLPEKLAQKNPMYQKERETPPRFAQHGTFEYEYSQRWKSLDEMEKQQREQVEKNMKDAKDKLESEMEDAYHEHQANLL

SEQ ID NO: 358 - protein, 234 aa, synthetic construct
  REGION 1..234: note = Synthetic: 4WIK
GAMAHAAALSVRNLSPYVSNELLEEAFSQFGPIERAVVIVDDRGRSTGKGIVEFASKPAARKAFERCSEGVFLLTTTPRPVIVEPLEQLDDEDGLPEKLAQKNPMYQKERETPPRFAQHGTFEYEYSQRWKSLDEMEKQQREQVEKNMKDAKDKLESEMEDAYHEHQANLLRQDLMRRQEELRRMEELHNQEMQKRKEMQLRQEEERRRREEEMMIRQREMEEQMRRQREESYS

SEQ ID NO: 359 - protein, 396 aa, synthetic construct
  REGION 1..396: note = Synthetic: 4WOT
AGASRQRKLEALIRDPRSPINVESLLDGLNSLVLDLDFPALRKNKNIDNFLNRYEKIVKKIRGLQMKAEDYDVVKVIGRGAFGEVQLVRHKASQKVYAMKLLSKFEMIKRSDSAFFWEERDIMAFANSPWVVQLFYAFQDDRYLYMVMEYMPGGDLVNLMSNYDVPEKWAKFYTAEVVLALDAIHSMGLIHRDVKPDNMLLDKHGHLKLADFGTCMKMDETGMVHCDTAVGTPDYISPEVLKSQGGDGFYGRECDWWSVGVFLYEMLVGDTPFYADSLVGTYSKIMDHKNSLCFPEDAEISKHAKNLICAFLTDREVRLGRNGVEEIRQHPFFKNDQWHWDNIRETAAPVVPELSSDIDSSNFDDIEDDKGDVETFPIPKAFVGNQLPFIGFTYYR

SEQ ID NO: 360 - protein, 87 aa, synthetic construct
  REGION 1..87: note = Synthetic: 4L2W
GIDPFTGNEGQMRELQDQLEAEQYFSTLYKTQVKELKEEIEEKNRENLKKIQELQNAAATLATQLDLAETKAESEQLARGLLEEQYF

SEQ ID NO: 361 - protein, 490 aa, synthetic construct
  REGION 1..490: note = Synthetic: 4L3I
GAAAMRRSEVLAEESIVCLQKALNHLREIWELIGIPEDQRLQRTEVVKKHIKELLDMMIAEEESLKERLIKSISVCQKELNTLCSELHVEPFQEEGETTILQLEKDLRTQVELMRKQKKERKQELKLLQEQDQELCEILCMPHYDIDSASVPSLEELNQFRQHVTTLRETKASRREEFVSIKRQIILCMEELDHTPDTSFERDVVCEDEDAFCLSLENIATLQKLLRQLEMQKSQNEAVCEGLRTQIRELWDRLQIPEEEREAVATIMSGSKAKVRKALQLEVDRLEELKMQNMKKVIEAIRVELVQYWDQCFYSQEQRQAFAPFCAEDYTESLLQLHDAEIVRLKNYYEVHKELFEGVQKWEETWRLFLEFERKASDPNRFTNRGGNLLKEEKQRAKLQKMLPKLEEELKARIELWEQEHSKAFMVNGQKFMEYVAEQWEMHRLEKERAKQERQLKNKKQTETEMLYGSAPRTPSKRRGLAPNTPGKAR

SEQ ID NO: 362 - protein, 402 aa, synthetic construct
  REGION 1..402: note = Synthetic: 4L6Q
GAAGDGAGASRQRKLEALIRDPRSPINVESLLDGLNSLVLDLDFPALRKNKNIDNFLNRYEKIVKKIRGLQMKAEDYDVVKVIGRGAFGEVQLVRHKASQKVYAMKLLSKFEMIKRSDSAFFWEERDIMAFANSPWVVQLFYAFQDDRYLYMVMEYMPGGDLVNLMSNYDVPEKWAKFYTAEVVLALDAIHSMGLIHRDVKPDNMLLDKHGHLKLADFGTCMKMDETGMVHCDTAVGTPDYISPEVLKSQGGDGFYGRECDWWSVGVFLYEMLVGDTPFYADSLVGTYSKIMDHKNSLCFPEDAEISKHAKNLICAFLTDREVRLGRNGVEEIRQHPFFKNDQWHWDNIRETAAPVVPELSSDIDSSNFDDIEDDKGDVETFPIPKAFVGNQLPFIGFTYYR

SEQ ID NO: 363 - protein, 56 aa, synthetic construct
  REGION 1..56: note = Synthetic: 4L9U
SELRHLRLPTYQELEQEINTLKADNDALKIQLKYAQKKIESLQLEKSNHVLAQMEQ

SEQ ID NO: 364 - protein, 219 aa, synthetic construct
  REGION 1..219: note = Synthetic: 4JMR
MAHHHHHHSAALEVLFQGPGMASGSNVEEYELDVEALVVILRDRNIPRNPLHGEVIGLRLTEGWWGQIERFQMVRLILQNDDNEPLQRPRYEVIQRAVNPHTMFMISGPLAELQLAFQDLDLPEGPLRFGPLANGHYVQGDPYSSSYRPVTMAETAQMTRDELEDVLNTQSEIEIQMINLLELYEVETRALRRQLAERSMAPPMTLQQWIIWKKMNKAH

SEQ ID NO: 365 - protein, 199 aa, synthetic construct
  REGION 1..199: note = Synthetic: 4JNH
MAHHHHHHSAALEVLFQGPGMASGSNVEEYELDVEALVVILRDRNIPRNPLHGEVIGLRLTEGWWGQIERFQMVRLILQNDDNEPLQRPRYEVIQRAVNPHTMFMISGPLAELQLAFQDLDLPEGPLRFGPLANGHYVQGDPYSSSYRPVTMAETAQMTRDELEDVLNTQSEIEIQMINLLELYEVETRALRRQLAERS

SEQ ID NO: 366 - protein, 78 aa, synthetic construct
  REGION 1..78: note = Synthetic: 4QQ4
  VARIANT 16: note = Xaa can be any naturally occurring amino acid
ARTKQTARKSTGGKAXGEDIQKRPDQTWVQCDACLKWRKLPDGMDQLPEKWYCSNNPDPQFRNCEVPEEPEDEDLVHP

SEQ ID NO: 367 - protein, 88 aa, synthetic construct
  REGION 1..88: note = Synthetic: 4R3Q
MADSDPGERSYDNMLKMLSDLNKDLEKLLEEMEKISVQATWMAYDMVVMRTNPTLAESMRRLEDAFLNCKEEMEKNWQELLTETKRKQ

SEQ ID NO: 368 - protein, 307 aa, synthetic construct
  REGION 1..307: note = Synthetic: 4HAN
GSMMLSLNNLQNIIYNPVIPFVGTIPDQLDPGTLIVIRGHVPSDADRFQVDLQNGSSVKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSFSSHMRLPFAARLNTPMGPGRTVVVKGEVNANAKSFNVDLLAGKSKDIALHLNPRLNIKAFVRNSFLQESWGEEERNITSFPFSPGMYFEMIIYCDVREFKVAVNGVHSLEYKHRFKELSSIDTLEINGDIHLLEVRSWPGLAYGNPYSGIQE

SEQ ID NO: 369 - protein, 166 aa, synthetic construct
  REGION 1..166: note = Synthetic: 4HHD
GSDDKVRQKEMRKGIDLATTLERIEKNFVITDPRLPDNPIIFASDSFLELTEYSREEILGRNCRFLQGPETDLTTVKKIRNAIDNQTEVTVQLINYTKSGKKFWNIFHLQPMRDQKGEVQYFIGVQLDGSKHVEPVRNVIEETAVKEGEDLVKKTAVNIDEAVREL

SEQ ID NO: 370 - protein, 35 aa, synthetic construct
  REGION 1..35: note = Synthetic: 4TL1
  VARIANT 1: note = Xaa can be any naturally occurring amino acid
  VARIANT 35: note = Xaa can be any naturally occurring amino acid
XRMKQLEDKVAELLSKNYHLENEVARLKKLVGERX

SEQ ID NO: 371 - protein, 276 aa, synthetic construct
  REGION 1..276: note = Synthetic: 4RSJ
AVVEDKRAEISEIEGKLSTIQAKRIKVEKEIEAKSNELEKVSKELESSERELIAAEAQREVRGNRAAEELKRSGIGGIYGTLAELIKVKDEAYALAIEVALGNRADNVVVEDELVAEKAIKYLKEHKLGRLTFLPLNKIKPKHVDSSVGLPAVDVIEYDQKIENAVKFALGDTVIVNSMEEARPHIGKVRMVTIEGELYERSGAITGGHFRARGLAVDTTKLREKVESLRRRKEALEGELNSLKIELRSLENASFELRIKLSDEKKELELASKDLN

SEQ ID NO: 372 - protein, 154 aa, synthetic construct
  REGION 1..154: note = Synthetic: 4XA3
GASMPLKPEEHEDILNKLLDPELAQSERTEALQQLRVNYGSFVSEYNDLTKSLSKANSEVAQWRTKYETDAIQRTEELEEAKKKLAQRLQEAEEAVEAVNAKCSSLEKTKHRLQNEIDFYFGKLRNIELICQENEGENDPVLQRIVDILYATDE

SEQ ID NO: 373 - protein, 209 aa, synthetic construct
  REGION 1..209: note = Synthetic: 4XA4
GGSGERKISRIHLVSEPSITHFLQVSWEKTLESGFVITLTDGHSAWTGTVSESEISQEADDMAMEKGKYVGELRKALLSGAGPADVYTFNFSKESCYFFFEKNLKDVSFRLGSFNLEKVENPAEVIRELICYCLDTTAENQAKNEHLQKELEHEEGKILRAQLEFNQIKAEIERKLAEKDEEMEQAKRNHLRVVDSLQTSLDAETRSRN

SEQ ID NO: 374 - protein, 410 aa, synthetic construct; identical to SEQ ID NO: 277
  REGION 1..410: note = Synthetic: 3V8S

SEQ ID NO: 375 - protein, 52 aa, synthetic construct
  REGION 1..52: note = Synthetic: 4DJG
ATSLEKHVLLKKLRDALESLRGRVAGRNKDDVEEAIAMVEALAVQLTQREGE

SEQ ID NO: 376 - protein, 106 aa, synthetic construct
  REGION 1..106: note = Synthetic: 4E61
GPLGSLVAIQAELTKSQETIGSLNEEIEQYKGTVSTLEIEREFYFNKLRDIEILVHTTQDLINEGVYKFNDETITGHGNGNGGALLRFVKKVESILYATAEGFEMN

SEQ ID NO: 377 - protein, 823 aa, synthetic construct; identical to SEQ ID NO: 314
  REGION 1..823: note = Synthetic: 3QX3

SEQ ID NO: 378 - protein, 455 aa, synthetic construct
  REGION 1..455: note = Synthetic: 3SJB
MDLTVEPNLHSLITSTTHKWIFVGGKGGVGKTTSSCSIAIQMALSQPNKQFLLISTDPAHNLSDAFGEKFGKDARKVTGMNNLSCMEIDPSAALKDMNDMAVSRANNNGSDGQGDDLGSLLQGGALADLTGSIPGIDEALSFMEVMKHIKRQEQGEGETFDTVIFDTAPTGHTLRFLQLPNTLSKLLEKFGEITNKLGPMLNSFMGAGNVDISGKLNELKANVETIRQQFTDPDLTTFVCVCISEFLSLYETERLIQELISYDMDVNSIIVNQLLFAENDQEHNCKRCQARWKMQKKYLDQIDELYEDFHVVKMPLCAGEIRGLNNLTKFSQFLNKEYNPITDGKVIYELEDKELEHHHHHHMQYTNKYHEKWISKFAPGNELSKKYLAKVKERHELKEFNNSISAQDNYAKWTKNNRKLDSLDKEINNLKDEIQSENKAFQAHLHKLEHHHHHH

SEQ ID NO: 379 - protein, 427 aa, synthetic construct
  REGION 1..427: note = Synthetic: 3SJC
MDLTVEPNLHSLITSTTHKWIFVGGKGGVGKTTSSCSIAIQMALSQPNKQFLLISTDPAHNLSDAFGEKFGKDARKVTGMNNLSCMEIDPSAALKDMNDMAVSRANNNGSDGQGDDLGSLLQGGALADLTGSIPGIDEALSFMEVMKHIKRQEQGEGETFDTVIFDTAPTGHTLRFLQLPNTLSKLLEKFGEITNKLGPMLNSFMGAGNVDISGKLNELKANVETIRQQFTDPDLTTFVCVCISEFLSLYETERLIQELISYDMDVNSIIVNQLLFAENDQEHNCKRCQARWKMQKKYLDQIDELYEDFHVVKMPLCAGEIRGLNNLTKFSQFLNKEYNPITDGKVIYELEDKELEHHHHHHMNELSKKYLAKVKERHELKEFNNSISAQDNYAKWTKNNRKLDSLDKEINNLKDEIQSENHHHHHH

SEQ ID NO: 380 - protein, 427 aa, synthetic construct; identical to SEQ ID NO: 379
  REGION 1..427: note = Synthetic: 3SJA

SEQ ID NO: 381 - protein, 191 aa, synthetic construct
  REGION 1..191: note = Synthetic: 3ME9
GRRGVLMTLLQQSAMTLPLWIGKPGDKPPPLCGAIPASGDYVARPGDKVAARVKAVDGDEQWILAEVVSYSHATNKYEVDDIDEEGKERHTLSRRRVIPLPQWKANPETDPEALFQKEQLVLALYPQTTCFYRALIHAPPQRPQDDYSVLFEDTSYADGYSPPLNVAQRYVVACKEPKKKARTKQTARKST

SEQ ID NO: 382 - protein, 194 aa, synthetic construct
  REGION 1..194: note = Synthetic: 3MEU
GRRGVLMTLLQQSAMTLPLWIGKPGDKPPPLCGAIPASGDYVARPGDKVAARVKAVDGDEQWILAEVVSYSHATNKYEVDDIDEEGKERHTLSRRRVIPLPQWKANPETDPEALFQKEQLVLALYPQTTCFYRALIHAPPQRPQDDYSVLFEDTSYADGYSPPLNVAQRYVVACKEPKKKARTKQTARKSTGGY

SEQ ID NO: 383 - protein, 188 aa, synthetic construct
  REGION 1..188: note = Synthetic: 3MEV
GRRGVLMTLLQQSAMTLPLWIGKPGDKPPPLCGAIPASGDYVARPGDKVAARVKAVDGDEQWILAEVVSYSHATNKYEVDDIDEEGKERHTLSRRRVIPLPQWKANPETDPEALFQKEQLVLALYPQTTCFYRALIHAPPQRPQDDYSVLFEDTSYADGYSPPLNVAQRYVVACKEPKKKAATKQTAR

SEQ ID NO: 384 - protein, 174 aa, synthetic construct
  REGION 1..174: note = Synthetic: 3LX7
MHHHHHHSSGRENLYFQGGDKPPPLCGAIPASGDYVARPGDKVAARVKAVDGDEQWILAEVVSYSHATNKYEVDDIDEEGKERHTLSRRRVIPLPQWKANPETDPEALFQKEQLVLALYPQTTCFYRALIHAPPQRPQDDYSVLFEDTSYADGYSPPLNVAQRYVVACKEPKKK

SEQ ID NO: 385 - protein, 95 aa, synthetic construct
  REGION 1..95: note = Synthetic: 3M91
SHAPTRSARDIHQLEARIDSLAARNSKLMETLKEARQQLLALREEVDRLGQSTAAGQERREKLTEETDDLLDEIDDVLEENAEDFVRAYVQKGGE

SEQ ID NO: 386 - protein, 46 aa, synthetic construct
  REGION 1..46: note = Synthetic: 2YMY
GSPELQNFLTILEKEEQDKIHQLQKKYNKFRQKLEEALRESQGKPG

SEQ ID NO: 387 - protein, 104 aa, synthetic construct
  REGION 1..104: note = Synthetic: 2Z17
GSSGSSGLSDFSWSQRKLVTVEKQDNETFGFEIQSYRPQNQNACSSEMFTLICKIQEDSPAHCAGLQAGDVLANINGVSTEGFTYKQVVDLIRSSGNLLTIETL

SEQ ID NO: 388 - protein, 287 aa, synthetic construct
  REGION 1..287: note = Synthetic: 3LLL
DSFWEVGNYKRTVKRIDDGHRLCGDLMNCLHERARIEKAYAQQLTEWARRWRQLVEKGPQYGTVEKAWIAVMSEAERVSELHLEVKASLMNEDFEKIKNWQKEAFHKQMMGGFKETKEAEDGFRKAQKPWAKKLKEVEAAKKAHHTACKEEKLAISREANSKADPSLNPEQLKKLQDKIEKCKQDVLKTKDKYEKSLKELDQTTPQYMENMEQVFEQCQQFEEKRLRFFREVLLEVQKHLDLSNVASYKTIYRELEQSIKAADAVEDLRWFRANHGPGMAMNWPQFE

SEQ ID NO: 389 - protein, 172 aa, synthetic construct
  REGION 1..172: note = Synthetic: 4E8U
SDCDHDEKLVYPWKGIVVNIPTTKAQDGRSAGESGSKLRDEYILRGFNPTRVRPLWNYLGHSGTAIVEFNKDWNGLHNGLLFDKAYTVDGHGKKDWLKKDGPKLGLYGWIARADDYNGNNIIGENLRKTGDLKTIAELTEEEARKQELLVQNLRQLVEEKKKDMKEIEELCS

SEQ ID NO: 390 - protein, 33 aa, synthetic construct
  REGION 1..33: note = Synthetic: 4DZM
  VARIANT 1: note = Xaa can be any naturally occurring amino acid
XGEIAALKQEIAALKKENAALKFEIAALKQGYY

SEQ ID NO: 391 - protein, 536 aa, synthetic construct
  REGION 1..536: note = Synthetic: 3ERR
MVDLKRLRQEPEVFHRAIREKGVALDLEALLAVDEQLHKQQEVIADKQMSVKEDLDKVEPAVIEAQNAVKSIKKQHLVEVRSMANPPAAVKLALESIALLLGESTTDWKQIRSIIMRENFIPTIVNFSAEEISDAIREKMKKNYMSNPSYNYEIVNRASLAAGPMVKWAIAQLNYADMLKRVEPLRNELQKLEDDAKDNQQKLEALLLQVPLPPWPGAPVGGEEANREIKRVGGPPEFSFPPLDHVALMEKNGWWEPRISQVSGSRSYALKGDLALYELALLRFAMDFMARRGFLPMTLPSYAREKAFLGTGHFPAYRDQVWAIAETDLYLTGTAEVVLNALHSGEILPYEALPLRYAGYAPAFRSEAGSFGKDVRGLMRVHQFHKVEQYVLTEASLEASDRAFQELLENAEEILRLLELPYRLVEVATGDMGPGKWRQVDIEVYLPSEGRYRETHSCSALLDWQARRANLRYRDPEGRVRYAYTLNNTALATPRILAMLLENHQLQDGRVRVPQALIPYMGKEVLEPGAHHHHHH

SEQ ID NO: 392 - protein, 163 aa, synthetic construct
  REGION 1..163: note = Synthetic: 4LPZ
GAMDNVCQPTEFISRHNIEGIFTFVDHRCVATVGYQPQELLGKNIVEFCHPEDQQLLRDSFQQVVKLKGQVLSVMFRFRSKNQEWLWMRTSSFTFQNPYSDEIEYIICTNTNVKNSSQEGEFEVNYHLENEVARLKKLVGEKTKEIDELTRICADLISKMAKI

SEQ ID NO: 393 - protein, 61 aa, synthetic construct
  REGION 1..61: note = Synthetic: 4M3L
GAMDTLYAILDEKKSELLQRITQEQEEKLSFIEALIQQYQEQLDKSTKLVETAIQSLDEPG

SEQ ID NO: 394 - protein, 529 aa, synthetic construct
  REGION 1..529: note = Synthetic: 4LRZ
GSSLSRTQIVNWLTRCGDIFSTESEYLTGLDREIGDADHGLNMNRGFSKVVEKLPAIADKDIGFILKNTGMTLLSSVGGASGPLFGTFFIRAAQATQARQSLTLEELYQMFRDGADGVISRGKAEPGDKTMCDVWVPVVESLRQSSEQNLSVPVALEAASSIAESAAQSTITMQARKGRASYLGERSIGHQDPGATSVMFMMQMLALAAKEMSGAFNNDGRGISPLIATSWERCNKLMKRETWNVPHQAQGVTFASIYRRKKAMLTLGQAALEDAWEYMAPRECALFILDETACILSRNGDPQTLQQLSALGFNDGTYCAEGIIGTCALSLAAISGQAVKTMADQHFKQVLWNWAFCATPLFDSKGRLTGTIALACPVEQTTAADLPLTLAIAREVGNLLLTDSLLAETNRHLNQLNALLESMDDGVISWDEQGNLQFINAQAARVLRLDATASQGRAITELLTLPAVLQQAIKQAHPLKHVEATFESQHQFIDAVITLKPIIETQGTSFILLLHPVEQMRQLMTSQLG

SEQ ID NO: 395 - protein, 191 aa, synthetic construct
  REGION 1..191: note = Synthetic: 4LTB
ASLSQASADLEATLRHKLTVMYSQINGASRALDDVRNRQQDVRMTANRKVEQLQQEYTEMKALLDASETTSTRKIKEEEKRVNSKFDTIYQILLKKKSEIQTLKEEIEQSLTKRDEFEFLEKASKLRGISTKPVYIPEVELNHKLIKGIHQSTIDLKNELKQCIGRLQELTPSSGDPGEHDPASTHKSTRP

SEQ ID NO: 396 - protein, 632 aa, synthetic construct
  REGION 1..632: note = Synthetic: 4M8M
GGSVKQLEDKVEELLSKNAHLENEVARLKKLQKQMSKKMNDQLELMESNIRRDIRQGFVDLQTEKSDLIDNVGAIPFLDYKHFASRIFFPEAGTLTAVMIRDIGEDSEQTTVDEKCLAFAELIRDKQFLSCFVHALEEQKNFSIKDKCTVASLLTLALHGDLLYLTEIMEDLLQSLMDQSSNANPKLLLRRTESIVEKLLTNWMSICLYGFLRESVGQPLFLLVSALTQQISKGPVDSVTEKALYTLSEDWLLCQAQDFEPLKLKVVFAVGTGEEISESLEVIALTCDTIQQVKEKILQTFQRKFGFRYTQQIRDIEIEYEKEGKFVMLQEVDDTSEIRGHVTMLNTLKHYQVGDGACIKVITPKIHAPLKTQNSVKDDKNFSIKYFHLVDPDIDTDLSNHPEKKALKIKEMYLIKLLSTKVAVHSFVENLFKSIWGLPNNKAPLAVKYFFDFLDEQAERKKITDPDVLHIWKTNSLPLRFWVNILKNPDFVFSDMEKSPHLDGCLSVIAQAFMDSFSLTDTHLDKHSPTNKLLYGKDIPQYKQEVKSYYKLVKDQTSISSQELKTFLQEESKKHQNEFNESAALRELYKYMQRYFTEIFQKLEQTDAPSNLKENMHRVKELFDNMKRSGWN

SEQ ID NO: 397 - protein, 154 aa, synthetic construct
  REGION 1..154: note = Synthetic: 4NAD
AMGSTEELIKALQDLENAASGDATVRQKIASLPQEVQDVSLLEKITDKEAAERLSKTVDEACLLLAEYNGRLAAELEDRRQLARMLVEYTQNQKDVLSEKEKKLEEYKQKLARVTQVRKELKSHIQSLPDLSLLPNVTGGLAPLPSAGDLFSTD

SEQ ID NO: 398 - protein, 50 aa, synthetic construct
  REGION 1..50: note = Synthetic: 4N6J
STMDWEVERAELQARIAMLQGERKGQENLKKDLVRRIKMLEMALKQERAK

SEQ ID NO: 399 - protein, 235 aa, synthetic construct
  REGION 1..235: note = Synthetic: 2QM4
SGMEELEQGLLMQPWAWLQLAENSLLAKVFITKQGYALLVSDLQQVWHEQVDTSVVSQRAKELNKRLTAPPAAFLCHLDNLLRPLLKDAAHPSEATFSCDCVADALILRVRSELSGLPFYWNFHCMLASPSLVSQHLIRPLMGMSLALQCQVRELATLLHMKDLEIQDYQESGATLIRDRLKTEPFEENSFLEQFMIEKLPEACSIGDGKPFVMNLQDLYMAVTTQEVQVGQKHQ

SEQ ID NO: 400 - protein, 179 aa, synthetic construct
  REGION 1..179: note = Synthetic: 2PON
GGTMENLSRRLKVTGDLFDIMSGMSQSNRELVVDFLSYKLSQKGYSWSQFSDVEENRTEAPEGTESEAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYG

SEQ ID NO: 401 - protein, 89 aa, synthetic construct
  REGION 1..89: note = Synthetic: 2Q2F
MHHHHHHSSGRENLYFQGSARLRALRQRQLDRAAAAVEPDVVVKRQEALAAARLKMQEELNAQVEKHKEKLKQLEEEKRRQKIEMWDSM

SEQ ID NO: 402 - protein, 178 aa, synthetic construct
  REGION 1..178: note = Synthetic: 1XJA
MAEAQNDPLLPGYSFNAHLVAGLTPIEANGVLDFFIDRPLGMKGYILNLTIRGQGVVKNQGREFVCRPGDILLFPPGEIHHYGRHPEAREWYHQWVYFRPRAYWHEWLNWPSIFANTGFFRPDEAHQPHFSDLFGQIINAGQGEGRYSELLAINLLEQLLLRRMEAINESLHPPMDNR

SEQ ID NO: 403 - protein, 415 aa, synthetic construct; identical to SEQ ID NO: 287
  REGION 1..415: note = Synthetic: 2ETR

SEQ ID NO: 404 - protein, 415 aa, synthetic construct; identical to SEQ ID NO: 287
  REGION 1..415: note = Synthetic: 2ETK

SEQ ID NO: 405 - protein, 156 aa, synthetic construct
  REGION 1..156: note = Synthetic: 2EUL
MAREVKLTKAGYERLMQQLERERERLQEATKILQELMESSDDYDDSGLEAAKQEKARIEARIDSLEDILSRAVILEEGSGEVIGLGSVVELEDPLSGERLSVQVVSPAEANVLDTPMKISDASPMGKALLGHRVGDVLSLDTPKGRREFRVVAIHG

SEQ ID NO: 406 - protein, 415 aa, synthetic construct; identical to SEQ ID NO: 287
  REGION 1..415: note = Synthetic: 2ESM

SEQ ID NO: 407 - protein, 175 aa, synthetic construct
  REGION 1..175: note = Synthetic: 2FV4
GSHMAAQSGNDAAEVALYERLLQLRVLPGASDVHDVRFVFGDDSRCWIEVAMHGDHVIGNSHPALDPKSRATLEHVLTVQGDLAAFLVVARDMLLASLMAKVIEPELEEQSAVTPEANENILKLKLYRSLGVILDLENDQVLINRKNDGNIDILPLDNNLSDFYKTKYIWERLGK

SEQ ID NO: 408 - protein, 129 aa, synthetic construct
  REGION 1..129: note = Synthetic: 2FXM
GSSPLLKSAEREKEMASMKEEFTRLKEALEKSEARRKELEEKMVSLLQEKNDLQLQVQAEQDNLADAEERCDQLIKNKIQLEAKVKEMNERLEDEEEMNAELTAKKRKLEDECSELKRDIDDLELTLAK

SEQ ID NO: 409 - protein, 67 aa, synthetic construct
  REGION 1..67: note = Synthetic: 1ZXA
GSPGIPGSTSELEEDFAKILMLKEERIKELEKRLSEKEEEIQELKRKLHKCQSVLPVPSTHIGPRTT

SEQ ID NO: 410 - protein, 68 aa, synthetic construct
  REGION 1..68: note = Synthetic: 2A93
  VARIANT 1: note = Xaa can be any naturally occurring amino acid
  VARIANT 34..35: note = Xaa can be any naturally occurring amino acid
  VARIANT 68: note = Xaa can be any naturally occurring amino acid
XCGGVQAEEQKLISEEDLLRKRREQLKHKLEQLXXCGGMRRKNDTHQQDIDDLKRQNALLEQQVRALX

SEQ ID NO: 411 - protein, 177 aa, synthetic construct
  REGION 1..177: note = Synthetic: 2AAC
AEAQNDPLLPGYSFNAHLVAGLTPIEANGYLDFFIDRPLGMKGYILNLTIRGQGVVKNQGREFVCRPGDILLFPPGEIHHYGRHPEAREWYHQWVYFRPRAYWHEWLNWPSIFANTGFFRPDEAHQPHFSDLFGQIINAGQGEGRYSELLAINLLEQLLLRRMEAINESLHPPMDNR

SEQ ID NO: 412 - protein, 149 aa, synthetic construct
  REGION 1..149: note = Synthetic: 2ARA
LVAGLTPIEANGYLDFFIDRPLGMKGYILNLTIRGQGVVKNQGREFVCRPGDILLFPPGEIHHYGRHPEAREWYHQWVYFRPRAYWHEWLNWPSIFANTGFFRPDEAHQPHFSDLFGQIINAGQGEGRYSELLAINLLEQLLLRRMEAI

SEQ ID NO: 413 - protein, 164 aa, synthetic construct
  REGION 1..164: note = Synthetic: 2ARC
DPLLPGYSFNAHLVAGLTPIEANGYLDFFIDRPLGMKGYILNLTIRGQGVVKNQGREFVCRPGDILLFPPGEIHHYGRHPEAREWYHQWVYFRPRAYWHEWLNWPSIFANTGFFRPDEAHQPHFSDLFGQIINAGQGEGRYSELLAINLLEQLLLRRMEAINES

SEQ ID NO: 414 - protein, 402 aa, synthetic construct
  REGION 1..402: note = Synthetic: 2H9V
GASGDGAGASRQRKLEALIRDPRSPINVESLLDGLNSLVLDLDFPALRKNKNIDNFLNRYEKIVKKIRGLQMKAEDYDVVKVIGRGAFGEVQLVRHKASQKVYAMKLLSKFEMIKRSDSAFFWEERDIMAFANSPWVVQLFCAFQDDKYLYMVMEYMPGGDLVNLMSNYDVPEKWAKFYTAEVVLALDAIHSMGLIHRDVKPDNMLLDKHGHLKLADFGTCMKMDETGMVHCDTAVGTPDYISPEVLKSQGGDGYYGRECDWWSVGVFLFEMLVGDTPFYADSLVGTYSKIMDHKNSLCFPEDAEISKHAKNLICAFLTDREVRLGRNGVEEIKQHPFFKNDQWNWDNIRETAAPVVPELSSDIDSSNFDDIEDDKGDVETFPIPKAFVGNQLPFIGFTYYR

SEQ ID NO: 415 - protein, 280 aa, synthetic construct
  REGION 1..280: note = Synthetic: 2GZD
MGTRDDEYDYLFKVVLIGDSGVGKSNLLSRFTRNEFNLESKSTIGVEFATRSIQVDGKTIKAQIWDTAGLERYRAITSAYYRGAVGALLVYDIAKHLTYENVERWLKELRDHADSNIVIMLVGNKSDLRHLRAVPTDEARAFAEKNGLSFIETSALDSTNVEAAFQTILTEIYGAMAAKFRASNIMPSSSFHMSPTSNEDLRKIPDSNPFDATAGYRSLTYEEVLQELVKHKELLRRKDTHIRELEDYIDNLLVRVMEETPSILRVPYEPSRKAGKFSNS

SEQ ID NO: 416 - protein, 280 aa, synthetic construct; identical to SEQ ID NO: 415
  REGION 1..280: note = Synthetic: 2GZH

SEQ ID NO: 417 - protein, 102 aa, synthetic construct
  REGION 1..102: note = Synthetic: 2HJD
MDLKDSVVSDTFELRPVIGLTRGLSSADIETLTANAIRLHRQLLEKADQLFQVLPDDIKIGTAAGGEQHLEYIEAMIEMHAQMSAVNTLVGLLGFIPKVSVN

SEQ ID NO: 418 - protein, 425 aa, synthetic construct
  REGION 1..425: note = Synthetic: 2DQ3
MIDINLIREKPDYVKERLATRDKELVSLVDKVLELDKRRREIIKRLEALRSERNKLSKEIGKLKREGKDTTEIQNRVKELKEEIDRLEEELRKVEEELKNTLLWIPNLPHPSVPVGEDEKDNVEVRRWGEPRKFDFEPKPHWEIGERLGILDFKRGAKLSGSRFTVIAGWGARLERALINFMLDLHTKKGYKEICPPHLVKPEILIGTGQLPKFEEDLYKCERDNLYLIPTAEVPLTNLYREEILKEENLPIYLTAYTPCYRREAGAYGKDIRGIIRQHQFDKVELVKIVHPDTSYDELEKLVKDAEEVLQLLGLPYRVVELCTGDLGFSAAKTYDIEVWFPSQNKYREISSCSNCEDFQARRMNTRFKDSKTGKNRFVHTLNGSGLAVGRTLAAILENYQQEDGSVVVPEVLRDYVGTDVIRPE

SEQ ID NO: 419 - protein, 455 aa, synthetic construct; identical to SEQ ID NO: 330
  REGION 1..455: note = Synthetic: 2DQ0

SEQ ID NO: 420 - protein, 135 aa, synthetic construct
  REGION 1..135: note = Synthetic: 2E7S
GPLGSLEEQLNKSLKTIASQKAAIENYNQLKEDYNTLKRELSDRDDEVKRLREDIAKENELRTKAEEEADKLNKEVEDLTASLFDEANNLVADARMEKYAIEILNKRLTEQLREKDMLLDTLTLQLKNLKKVMHS

SEQ ID NO: 421 - protein, 402 aa, synthetic construct; identical to SEQ ID NO: 414
  REGION 1..402: note = Synthetic: 2F2U

SEQ ID NO: 422 - protein, 71 aa, synthetic construct
  REGION 1..71: note = Synthetic: 1UIX
GSTSDVANLANEKEELNNKLKEAQEQLSRLKDEEISAAAIKAQFEKQLLTERTLKTQAVNKLAEIMNRKEP

SEQ ID NO: 423 - protein, 83 aa, synthetic construct
  REGION 1..83: note = Synthetic: 1UII
PESSENKNLGGVTQESFDLMIKENPSSQYWKEVAEKRRKALYEALKENEKLHKEIEQKDNEIARLKKENKELAEVAEHVQYMA

SEQ ID NO: 424 - protein, 244 aa, synthetic construct
  REGION 1..244: note = Synthetic: 1URU
MTENKGIMLAKSVQKHAGRAKEKILQNLGKVDRTADEIFDDHLNNFNRQQASANRLQKEFNNYIRCVRAAQAASKTLMDSVCEIYEPQWSGYDALQAQTGASESLWADFAHKLGDQVLIPLNTYTGQFPEMKKKVEKRNRKLIDYDGQRHSFQNLQANANKRKDDVKLTKGREQLEEARRTYEILNTELHDELPALYDSRILFLVTNLQTLFATEQVFHNETAKIYSELEAIVDKLATESQRGS

SEQ ID NO: 425 - protein, 102 aa, synthetic construct
  REGION 1..102: note = Synthetic: 1RFY
MELEDANVTKKVELRPLIGLTRGLPPTDLETITIDAIRTHRRLVEKADELFQALPETYKTGQACGGPQHIRYIEASIEMHAQMSALNTLISILGFIPKVVVN

SEQ ID NO: 426 - protein, 254 aa, synthetic construct
  REGION 1..254: note = Synthetic: 1S1C
GSMAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQAGSMLTKDIEILRRENEELTEKMKKAEEEYKLEKEEEISNLKAAFEKNINTERTLKTQAVNKLAEIMNRKDF

SEQ ID NO: 427 - protein, 166 aa, synthetic construct
  REGION 1..166: note = Synthetic: 1NYH
GPLGSKRVPIVEDRRVKQLDDITDSNTTEILTSVDVLGTHSQTGTQQSNMYTSTQKTELEIDNKDSVTECSKDMKEDGLSFVDIVLSKAASALDEKEKQLAVANEIIRSLSDEVMRNEIRITSLQGDLTFTKKCLENARSQISEKDAKINKLMEKDFQVNKEIKPY

SEQ ID NO: 428 - protein, 50 aa, synthetic construct
  REGION 1..50: note = Synthetic: 1OV9
SEITKTLLNIRSLRAYARELTIEQLEEALDKLTTVVQERKEAEAEEIAAR

SEQ ID NO: 429 - protein, 34 aa, synthetic construct
  REGION 1..34: note = Synthetic: 2NRN
MKVKQLADKVEELLSKNYHLANEVARLAKLVGER

SEQ ID NO: 430 - protein, 107 aa, synthetic construct
  REGION 1..107: note = Synthetic: 2NO2
GSHADLLRKNAEVTKQVSMARQAQVDLEREKKELEDSLERISDQGQRKTQEQLEVLESLKQELATSQRELQVLQGSLETSAQSEANWAAEFAELEKERDSLVSGAAH

SEQ ID NO: 431 - protein, 69 aa, synthetic construct
  REGION 1..69: note = Synthetic: 2N9B
GSHENKQVEEILRLEKEIEDLQRMKERQELSLTEASLQKLQLEDKVEELLSKNYHLENEVARLKKLVGE

SEQ ID NO: 432 - protein, 79 aa, synthetic construct
  REGION 1..79: note = Synthetic: 2L2L
GSPEERERMIKQLKEELRLEEAKLVLLKKLRQSQIQKEATAQKGSKAFIVTDEDIRKQEERVQQVRKKLEEALMADILS

SEQ ID NO: 433 - protein, 51 aa, synthetic construct
  REGION 1..51: note = Synthetic: 2LW9
ENKQVEEILRLEKEIEDLQRMKEQQELSLTEASLQKLQERRDQELRRLEEE

SEQ ID NO: 434 - protein, 105 aa, synthetic construct
  REGION 1..105: note = Synthetic: 2MAK
GSHMASSRQKYAEEELEQVREALRKAEKELESHSSWYAPEALQKWLQLTHEVEVQYYNIKKQNAEKQLLVAKEGAEKIKKKRGSELNELAEFARLQDQLDHRGDH

SEQ ID NO: 435 - protein, 82 aa, synthetic construct
  REGION 1..82: note = Synthetic: 2MAJ
GSHMASSRQKYAEEELEQVREALRKAEKELESHSSWYAPEALQKWLQLTHEVEVQYYNIKKQNAEKQLLVAKEGAEKIKKKR

SEQ ID NO: 436 - protein, 81 aa, synthetic construct
  REGION 1..81: note = Synthetic: 2JEE
MTMSLEVFEKLEAKVQQAIDTITLLQMEIEELKEKNNSLSQEVQNAQHQREELERENNHLKEQQNGWQERLQALLGRMEEV

SEQ ID NO: 437 - protein, 41 aa, synthetic construct
  REGION 1..41: note = Synthetic: 2K6S
GSLTYEEVLQELVKHKELLRRKDTHIRELEDYIDNLLVRVM

SEQ ID NO: 438 - protein, 35 aa, synthetic construct
  REGION 1..35: note = Synthetic: 4HU5
  VARIANT 1: note = Xaa can be any naturally occurring amino acid
  VARIANT 8: note = Xaa can be any naturally occurring amino acid
  VARIANT 12: note = Xaa can be any naturally occurring amino acid
  VARIANT 35: note = Xaa can be any naturally occurring amino acid
XRLKQLEXKVEXLLSKNYHLENEVARLKKLVGERX

SEQ ID NO: 439 - protein, 35 aa, synthetic construct; identical to SEQ ID NO: 438
  REGION 1..35: note = Synthetic: 4HU6
  VARIANT 1: note = Xaa can be any naturally occurring amino acid
  VARIANT 8: note = Xaa can be any naturally occurring amino acid
  VARIANT 12: note = Xaa can be any naturally occurring amino acid
  VARIANT 35: note = Xaa can be any naturally occurring amino acid

SEQ ID NO: 440 - protein, 104 aa, synthetic construct
  REGION 1..104: note = Synthetic: 4HW0
MQLERRKRGTMEIMFDILRNCEPKCGITRVIYGAGINYVVAQKYLDQLVKVGALNIKTENDRKIYEITEKGKLLRTHIEEFIKIRENLYSAKEKVSELLRTDSE

SEQ ID NO: 441 - protein, 823 aa, synthetic construct; identical to SEQ ID NO: 314
  REGION 1..823: note = Synthetic: 4J3N

SEQ ID NO: 442 - protein, 466 aa, synthetic construct
  REGION 1..466: note = Synthetic: 5IX1
STGIRLSALCPKFLHTNSTSHTWPFSAVAELIDNAYDPDVNAKQIWIDKTVISDHICLTFTDNGNGMTADKLHKMLSFGFSDKVTMNGHVPVGLYGNGFKSGSMRLGKDAMVFTKNGETMSVGFLSQTYLEVIKAEHVVVPIVTFNKHRQMINLTESKASLAAILEHSLFSTEQKLLAELNAIMGKKGTRIIIWNLRSYKNATEFDFEKDKYDIRIPEDLDETAGRKGYKKQERMDQIAPESDYSLRAYCSILYLKPRMQIIIRGQKVKTQLVSKSLAYIERDVYRPKFLTRTVRITFGFNCRNKDHYGIMMYHKNRLIKAYEKVGCQLKANNMGVGVVGIIECNFLKPTHNKQDFDYTNEYRLTILALGEKLNDYWNEMKVKKNAEYPVNLPVEDIQKRPDQTWVQCDACLKWRKLPDGIDQLPEKWYCSNNPDPQFRNCEVPEEPEDEDARTKQTARKSTGGKA

SEQ ID NO: 443 - protein, 483 aa, synthetic construct
  REGION 1..483: note = Synthetic: 5IX2
STGIRLSALCPKFLHTNSTSHTWPFSAVAELIDNAYDPDVNAKQIWIDKTVISDHICLTFTDNGNGMTADKLHKMLSFGFSDKVTMNGHVPVGLYGNGFKSGSMRLGKDAMVFTKNGETMSVGFLSQTYLEVIKAEHVVVPIVTFNKHRQMINLTESKASLAAILEHSLFSTEQKLLAELNAIMGKKGTRIIIWNLRSYKNATEFDFEKDKYDIRIPEDLDETAGRKGYKKQERMDQIAPESDYSLRAYCSILYLKPRMQIIIRGQKVKTQLVSKSLAYIERDVYRPKFLTRTVRITFGFNCRNKDHYGIMMYHKNRLIKAYEKVGCQLKANNMGVGVVGIIECNFLKPTHNKQDFDYTNEYRLTILALGEKLNDYWNEMKVKKNAEYPVNLPVEDIQKRPDQTWVQCDACLKWRKLPDGIDQLPEKWYCSNNPDPQFRNCEVPEEPEDEDARTKQTARKSTGGKAPRKQLATKAARKSAPAT

SEQ ID NO: 444 - protein, 677 aa, synthetic construct
  REGION 1..677: note = Synthetic: 5JHF
MNSENTIVYVRVAGRARNGFVDPLKFYWDLERDRSLWSSVSKLDNTKKTIDWKRLSREFKAPEHFIRKRSYALFAKHLKLLERQIEAMSSEANPPVLEPFTVTVVDRNVKHQVEGEPEEEGHPDHEVQGVMFATNVKYIFEDDQELLPEQEDPAIENVVIIEADESLRVTQVELISDQFKQVGYEVRDGNEVCIDALSRFETPRQLGNLPLEKLVQLYKLQNDQLHSLFNTLHHHHHHMNEAVIEKLLENSRKFLTGAKLICQESNDHLTTTKLRIREWQKFQSKLHFVLDCIQQQTKFLSEILLREGIGRNLIEEEWSQTVLVRLVNDMKFWQNEITKMMNKLDNITNEIDQQHNSKLGDFISRDSSHILDSKLNEIPTIRKQVENITRQYQTMLAKVQSQLVESRMKGLRDEFSSKFGDQCRENLKLNEEFTNEADQLEQELADFLKSFTDHFDKCSALSSRSVSPEDAQNLFEIVERDDKDLAAINSLLQDAAIDVASFVRKVNMLLDERDADKAKMQATLSKLLTELRKHEEYISVFEGISALIQKFKASCLEDIRQTRNLLDFYANFERSYHNLLKEVKRRKETAAKLSQILKSCETQLEQINTADLRERQMFLLENGNYLPETIWPDEIGSLSPLYTLNYEVRKVSKYSSSFGRLRRQLQPFKAGSVGSGS

SEQ ID NO: 445 - protein, 75 aa, synthetic construct
  REGION 1..75: note = Synthetic: 5JVR
LSNEDPKDALAEEWKRRYEKEKEKVEDLEKERDFYFGKLRNIELICQENEGENDPVLQRIVDILYATDEGFVIPD

SEQ ID NO: 446 - protein, 88 aa, synthetic construct
  REGION 1..88: note = Synthetic: 5JVS
LSKNVVAVNEELTDALAEEWKRRYEKEKEKNARLKGKVEDLEKERDFYFGKLRNIELICQENEGENDPVLQRIVDILYATDEGFVIPD

SEQ ID NO: 447 - protein, 85 aa, synthetic construct
  REGION 1..85: note = Synthetic: 5JVU
LSKNVVAVNEELTAEEWKRRYEKEKEKNARLKGKVEDLEKERDFYFGKLRNIELICQENEGENDPVLQRIVDILYATDEGFVIPD

SEQ ID NO: 448 - protein, 82 aa, synthetic construct
  REGION 1..82: note = Synthetic: 5JVM
LSNEDPKDTLLREFQEEIARLKAQLEKKGMLVEDLEKERDFYFGKLRNIELICQENEGENDPVLQRIVDILYATDEGFVIPD

SEQ ID NO: 449 - protein, 90 aa, synthetic construct
  REGION 1..90: note = Synthetic: 5JVP
LSNEVSTDEALLKRYRKEIMDLKKQLEEVSLETRAQAMEKDQLEKERDFYFGKLRNIELICQENEGENDPVLQRIVDILYATDEGFVIPD

SEQ ID NO: 450 - protein, 75 aa, synthetic construct
  REGION 1..75: note = Synthetic: 5JX1
LSNEDPKDALLRQFQKEIEELKKKLEELEKERDFYFGKLRNIELICQENEGENDPVLQRIVDILYATDEGFVIPD

SEQ ID NO: 451 - protein, 65 aa, synthetic construct
  REGION 1..65: note = Synthetic: 5FCN
GSNNELYLELMKLREHSDQHVKELKTSLKKCARETADLKFLNNQYAHKLKLLEKESKAKNERIQQ

SEQ ID NO: 452 - protein, 104 aa, synthetic construct
  REGION 1..104: note = Synthetic: 5FIY
ASWSHPQFEKAGFSRHNRDTSAPANFASSSTLEKRIEDLEKEVLRERQENLRLTRLMQDKEEMIGKLKEEIDLLNRDLDDMEDENEQLKQENKTLLKVVGQLTR

SEQ ID NO: 453 - protein, 148 aa, synthetic construct
  REGION 1..148: note = Synthetic: 4YTO
DTYQKEIEDKKISEENLLEEVEKAKVIADEAVKLQKEIDKRCQHKIAEMVALMEKHKHQYDKIIEERDSELGLYKSKEQEQSSLRASLEIELSNLKAELLSVKKQLEIEREEKEKLKREAKENTATLKEKKDKKTQTFLLLEHHHHHH

SEQ ID NO: 454 - protein, 415 aa, synthetic construct; identical to SEQ ID NO: 287
  REGION 1..415: note = Synthetic: 4YVC

SEQ ID NO: 455 - protein, 415 aa, synthetic construct; identical to SEQ ID NO: 287
  REGION 1..415: note = Synthetic: 4YVE

SEQ ID NO: 456 - protein, 329 aa, synthetic construct
  REGION 1..329: note = Synthetic: 4Z6Y
GVEEKKSLEILLKDDRLDTEKLCTFSQRFPLPSMYRALVWKVLLGILPPHHESHAKVMMYRKEQYLDVLHALKVVRFVSDATPQAEVYLRMYQLESGKLPRSPSFPLEPDDEVFLAIAKAMEEMVEDSVDCYWITRRFVNQLNTKYRDSLPQLPKAFEQYLNLEDGRLLTHLRMCSAAPKLPYDLWFKRCFAGCLPESSLQRVWDKVVSGSCKILVFVAVEILLTFKIKVMALNSAEKITKFLENIPQDSSDAIVSKAIDLWHKHCGTPVHSSRGQLQAAESRYEAQKRITQVFELEILDLYGRLEKDGLLKKLEEEKAEAAEAAEERL

SEQ ID NO: 457 - protein, 458 aa, synthetic construct
  REGION 1..458: note = Synthetic: 4ZI3
MAAEEEDEVEWVVESIAGFLRGPDWSIPILDFVEQKCEVFDDEEESKLTYTEIHQEYKELVEKLLESYLKEIGINEDQFQEACTSPLAKTRTSQAILQPVLAAEDFTIFKAMMVQKNIEMQLQAIRIIQERNGMGLLSILRKLKSAPDQEVRILLLGLDNAGKTTLLKQLASEDISHITPTQGFNIKSVQSQGFKLNVWDIGGQRKIRPYWRSYFENTDILIYVIDSADRKRFEETGQELTELLEEEKLSCVPVLIFANKQDLLTAAPASEIAEGLNLHTIRDRVWQIQSCSALTGEGVQDGMNWVCKNVNAKKKLEHHHHHHGPMAAEEEDEVEWVVESIAGFLRGPDWSIPILDFVEQKCEVFDDEEESKLTYTEIHQEYKELVEKLLESYLKEIGINEDQFQEACTSPLAKTRTSQAILQPVLAAEDFTIFKAMMVQKNIEMQLQAIRIIQERNG

SEQ ID NO: 458 - protein, 86 aa, synthetic construct
  REGION 1..86: note = Synthetic: 4ZQA
SNAGTLQEYQKRMKKLDQQYRERIRNAELFLQLETEQVERNYIKEKKAAVKEFEDKKVELKENLIAELEEKKKMIENEKLTMELTG

SEQ ID NO: 459 - protein, 68 aa, synthetic construct
  REGION 1..68: note = Synthetic: 5AJS
SLSSGTTEEELLRKLNEQRDILALMEVKMKEMKGSIRHLRLTEAKLREELREKDRLLAMAVIRKKHGM

SEQ ID NO: 460 - protein, 219 aa, synthetic construct
  REGION 1..219: note = Synthetic: 5AL7
GPMGWPPGSEDSYSAKMDYGKSVVNILPSVEMLVNFNGDMTRSSKRSCLLYAERVDFKELLQLRLTEKSDQRRMYITTVDSASFQDLKQDQSLNVSFSGFIDNVVRMLKDCQSGKLELHLTTRDQNLSSGREVHDYYLQFVEIRSDKNLVHLSLPCRSAPLNTVLFYINSMLEASHKKQYILEQSMQQMQAEINAQRAHAERLTTENTNLREALAENTR

SEQ ID NO: 461 - protein, 244 aa, synthetic construct
  REGION 1..244: note = Synthetic: 4ZRY
GSHMEAWFNEKSKELTTEIDNNIEQISSYKSEITELRRNVQALEIELQSQLALKQSLEASLAETEGRYCVQLSQIQAQISALEEQLQQIRAETECQNTEYQQLLDIKIRLENEIQTYRSLLEGEESLYQSKYEELQITAGRHGDSVRNSKIEISELNRVIQRLRSEIDNVKKQISNLQQSISDAEQRGENALKDAKNKLNDLEDALQQAKEDLARLLRDYQELMNTKLALDLEIATYRTLLEGE

SEQ ID NO: 462 - protein, 415 aa, synthetic construct; identical to SEQ ID NO: 287
  REGION 1..415: note = Synthetic: 5BML

SEQ ID NO: 463 - protein, 471 aa, synthetic construct
  REGION 1..471: note = Synthetic: 5AMO
MITNWMSQTLPSLVGLNTTRLSAASGGTLDRSTGVLPTNPEESWQVYSSAQDSEGRCICTVVAPQQTMCSRDARTKQLRQLLEKVQNMSQSIEVLDRRTQRDLQYVEKMENQMKGLETKFKQVEESHKQHLARQFKAIKAKMDELRPLIPVLEEYKADAKLVLQFKEEVQNLTSVLNELQEEIGAYDYDELQSRVSNLEERLRACMQKLACGKLTGISDPVTVKTSGSRFGSWMTDPLAPEGDNRVWYMDGYHNNRFVREYKSMVDFMNTDNFTSHRLPHPWSGTGQVVYNGSIYFNKFQSHIIIRFDLKTETILKTRSLDYAGYNNMYHYAWGGHSDIDLMVDENGLWAVYATNQNAGNIVISKLDPVSLQILQTWNTSYPKRSAGEAFIICGTLYVTNGYSGGTKVHYAYQTNASTYEYIDIPFQNKYSHISMLDYNPKDRALYAWNNGHQTLYNVTLFHAAAHHHHHH

SEQ ID NO: 464 - protein, 94 aa, synthetic construct
  REGION 1..94: note = Synthetic: 5DJN
GPGSHAVVNEDPNAKVIRELREEVEKLREQLSQAEAMKAELKEKLEESEKLIKELTVTWEEKLRKTEAIAQERQRQLESMGISLETSGIKVGDD

SEQ ID NO: 465 - protein, 174 aa, synthetic construct
  REGION 1..174: note = Synthetic: 5DJO
GPGSAMKAPELKEKLEESEKLIKELTVTWEEKLRKTEAIAQERQRQLESMGISLETSGIKVGDDKCYLVNLNADPALNELLVYYLKDHTRVGADTSQDIQLFGIGIQPEHCEIDIAADGDITLTPKENARSCVNGTLVCSTTQLWHGDRILWGNNHFFRINLPKRKRRDWLKDF

SEQ ID NO: 466 - protein, 85 aa, synthetic construct
  REGION 1..85: note = Synthetic: 5C9N
DSNFPLPDLCSWEEAQLSSQLYRNKQLQDTLVQKEEELARLHEENNHLRQYLNSALVKCEEEKAKKELSSDEFSKAYGKFRKGKR

SEQ ID NO: 467 - protein, 167 aa, synthetic construct
  REGION 1..167: note = Synthetic: 5CFF
GPGSEFELRRQASNYQLTLTNTRATVNILMERLKKSDADVEQYRAELESVQLAKGALEQSYLVLQADAEQLRQQLTESQDALNALRSSSQTLQSEGPGSMKEQLLYLSKLLDFEVNFSDYPKGNHNEFLTIVTLSTHPPQICHGVGKSSEESQNDAASNALKILSKL

SEQ ID NO: 468 - protein, 214 aa, synthetic construct
  REGION 1..214: note = Synthetic: 5CHX
GGSGERKISRIHLVSEPSITHFLQVSWEKTLESGFVITLTDGHSAWTGTVSESEISQEADDMAMEKGKYVGELRKALLSGAGPADVYTFNFSKESCYFFFEKNLKDVSFRLGSFNLEKVENPAEVIRELICYCLDTTAENQAKNEHHLRVVDSLQTSLDAETRSRNEALRVKKKMEGDLNEMEIQLSHANRMAAEAQKQVKSLQSLLKDTQIQL

SEQ ID NO: 469 - protein, 208 aa, synthetic construct
  REGION 1..208: note = Synthetic: 5CJ4
GGSGERKISRIHLVSEPSITHFLQVSWEKTLESGFVITLTDGHSAWTGTVSESEISQEADDMAMEKGKYVGELRKALLSGAGPADVYTFNFSKESCYFFFEKNLKDVSFRLGSFNLEKVENPAEVIRELICYCLDTTAENQAKNEHLQLEFNQIKAEIERKLAEKDEEMEQAKRNHLRVVDSLQTSLDAETRSRNEALRVKKKMEGDL

SEQ ID NO: 470 - protein, 101 aa, synthetic construct
  REGION 1..101: note = Synthetic: 5CJ1
GGSGPLKPEEHEDILNKLLDPELAQSERTEALQQLRVNYGSFVSEYNDLTKSHEKLEKVRKQLEAEKMELQSALEEAEASLEHEEGKILRAQLEFNQIKAE

SEQ ID NO: 471 - protein, 207 aa, synthetic construct
  REGION 1..207: note = Synthetic: 5CJ0
GGSGERKISRIHLVSEPSITHFLQVSWEKTLESGFVITLTDGHSAWTGTVSESEISQEADDMAMEKGKYVGELRKALLSGAGPADVYTFNFSKESCYFFFEKNLKDVSFRLGSFNLEKVENPAEVIRELICYCLDTTAENQAKNEHANRMAAEAQKQVKSLQSLLKDTQIQLDDAVRANDDLKENIAIVERRNNLLQAELEELRAVV

SEQ ID NO: 472 - protein, 82 aa, synthetic construct
  REGION 1..82: note = Synthetic: 5D3A
GSMTISNMEADMNRLLKQREELTKRREKLSKRREKIVKENGEGDKNVANINEEMESLTANIDYINDSISDCQANIMQMEEAK

SEQ ID NO: 473 - protein, 94 aa, synthetic construct
  REGION 1..94: note = Synthetic: 4BL6
GSHMYENEKIIVSDTMSKLRNELRLLKEDAATFSSLRAMFAARCEEYVTQVDDLNRQLEAAEEEKKTLNQLLRLAVQQKLALTQRLEEMEMDRE

SEQ ID NO: 474 - protein, 36 aa, synthetic construct
  REGION 1..36: note = Synthetic: 4C1A
GPAMEALELELEEVESQIRALVVRRSRLRERLLAVP

SEQ ID NO: 475 - protein, 171 aa, synthetic construct
  REGION 1..171: note = Synthetic: 4C1B
GPAMDVAIIGDSIVRHVRAASSKGNKVRTFCFPGARVKNISTQIPTILGAAESPGAVVLHVGTNDTGLRQSEILKKDFRSLIETVRRTSPATQIIVSGPLPTYRRGNERFSRLLALNEWLITWCKEQKLLFANNWNLFWERPRLFRPDGLHPSRAGAELLSDNISRLLRTI

SEQ ID NO: 476 - protein, 238 aa, synthetic construct
  REGION 1..238: note = Synthetic: 5F3K
AGHSTQTAEDKEEPLHSIISSTESVQGSTSKHEFQAETKKLLDIVARSLYSEKEVFIRELISNASDALEKLRHKLVSDGQALPEMEIHLQTNAEKGTITIQDTGIGMTQEELVSNLGTIARSGSKAFLDALQNQAEASSKIIGQFGVGFYSAFMVADRVEVYSRSAAPGSLGYQWLSDGSGVFEIAEASGVRTGTKIIIHLKSDCKEFSSEARVRDVVTKYSNFVSFPLYLNGRRMNT

SEQ ID NO: 477 - protein, 184 aa, synthetic construct
  REGION 1..184: note = Synthetic: 5F4Y
SDLDHDLSVKKQELIESISRKLQVLREARESLLEDVQANTVLGAEVEAIVKGVCKPSEFDKFRMFIGDLDKVVNLLLSLSGRLARVENALNNLDDGASPGDRQSLLEKQRVLIQQHEDAKELKENLDRRERIVFDILANYLSEESLADYEHFVKMKSALIIEQRELEDKIHLGEEQLKCLLDSL

SEQ ID NO: 478 - protein, 238 aa, synthetic construct; identical to SEQ ID NO: 476
  REGION 1..238: note = Synthetic: 5F5R

SEQ ID NO: 479 - protein, 314 aa, synthetic construct
  REGION 1..314: note = Synthetic: 5EYA
MAGLPRRIIKETQRLLAEPVPGIKAEPDESNARYFHVVIAGPQDSPFEGGTFKLELFLPEEYPMAAPKVRFMTKIYHPNVDKLGRIKLDILKDKWSPALQIRTVLLSIQALLSAPNPDDPLANDVAEQWKTNEAQAIETARAWTRLYAMNNIGSHMAELCPLAEELSCSICLEPFKEPVTTPCGHNFCGSCLNETWAVQGSPYLCPQCRAVYQARPQLHKNTVLCNVVEQFLQADLARMQIFVKTLTGKTITLEVEPSDTIENVKAKIQDKEGIPPDQQRLIFAGKQLEDGRTLSDYNIQKESTLHLVLRLRGG

SEQ ID NO: 480 - protein, 77 aa, synthetic construct
  REGION 1..77: note = Synthetic: 5M97
SGSAKQAQQQITSLETQLYEVNETMFGLERERDFYFNKLREIEILVQTHLTTSPMSMENMLERIQAILYSTEDGFEL

SEQ ID NO: 481 - protein, 97 aa, synthetic construct
  REGION 1..97: note = Synthetic: 5M9E
SGSAKQAQQQITSLETQLYEVNETMFGLERERDFYFNKLREIEILVQTHLTTSPMSMENMLERIQAILYSTEDGFELRRSLAGSMLQKPTQFSRPSF

SEQ ID NO: 482 - protein, 93 aa, synthetic construct
  REGION 1..93: note = Synthetic: 5HHE
DDSEQLQMELKELALEEERLIQELEDVEKNRKIVAENLEKVQAEAERLDQEEAQYQREYSEFKRQQLELDDELKSVENQMRYAQTQLDKLKLE

SEQ ID NO: 483 - protein, 134 aa, synthetic construct
  REGION 1..134: note = Synthetic: 5HMO
QKQLRGQIARRVYRQLLAEKRAEEEKRKREEEEKRKREEEERERERERREAELRAQQEEAARKQRELEALQQESQRAAELSRELEKQKENKQVEEILRLEKEIEDLQRMKERQELSLTEASLQKLQQLRDEELR

SEQ ID NO: 484 - protein, 43 aa, synthetic construct
  REGION 1..43: note = Synthetic: 4GDO
GSHMRAEERERLAEVEAALEKQRQLAEAHAQAKAQAEREAKEL

SEQ ID NO: 485 - protein, 189 aa, synthetic construct
  REGION 1..189: note = Synthetic: 4GFC
GSMTSKIALFDQTLIASLLQPLSLNQPDFKAYKTKVKLKISEQRNETSGEKELKFEISRSDDFEFLFSETLNNEKYQILARDHDLTVDFDAFPKVIIQHLLCKNTEINIILDAEKNFCSFELFSKTPGSKGKIFSIKLHAVRGDHLISHLLKICSSQAVKLSTFYKSADELASLRQKCGDLEKQVEKLS

SEQ ID NO: 486 - protein, 183 aa, synthetic construct
  REGION 1..183: note = Synthetic: 4GFA
GSMTSKIALFDQTLIASLLQPLSLNQPDFKAYKTKVKLKISEQRNETSGEKELKFEISRSDDFEFLFSETLNNEKYQILARDHDLTVDFDAFPKVIIQHLLCKNTEINIILDAEKNFCSFELFSGKIFSIKLHAVRGDHLISHLLKICSSQAVKLSTFYKSADELASLRQKCGDLEKQVEKLS

SEQ ID NO: 487 - protein, 168 aa, synthetic construct
  REGION 1..168: note = Synthetic: 4GEU
MTSKIALFDQTLIASLLQPLSLNQPDFKAYKTKVKLKISEQRNETSGEKELKFEISRSDDFEFLFSETLNNEKYQILARDHDLTVDFDAFPKVIIQHLLCKNIVKNLEEDGEVDARKKAGYHEIADPGKPTEINIILDAEKNFCSFELFSKTPWSKGKIFSIKLHAVR

SEQ ID NO: 488 - protein, 170 aa, synthetic construct
  REGION 1..170: note = Synthetic: 4GEX
GSMTSKIALFDQTLIASLLQPLSLNQPDFKAYKTKVKLKISEQRNETSGEKELKFEISRSDDFEFLFSETLNNEKYQILARDHDLTVDFDAFPKVIIQHLLCKNIVKNLEEDGEVDARKKAGYHEIADPGKPTEINIILDAEKNFCSFELFSKTPWSKGKIFSIKLHAVR

SEQ ID NO: 489 - protein, 68 aa, synthetic construct; identical to SEQ ID NO: 410
  REGION 1..68: note = Synthetic: 1A93
  VARIANT 1: note = Xaa can be any naturally occurring amino acid
  VARIANT 34..35: note = Xaa can be any naturally occurring amino acid
  VARIANT 68: note = Xaa can be any naturally occurring amino acid

SEQ ID NO: 490 - protein, 48 aa, synthetic construct
  REGION 1..48: note = Synthetic: 5HUZ
DFKKLYEQILAENEKLKAQLHDTNMELTDLKLQLEKATQRQERFADRS

SEQ ID NO: 491 - protein, 251 aa, synthetic construct
  REGION 1..251: note = Synthetic: 1JAD
GAMDGNMKEVTQLPEPQTASLAELQQMKLFLKLLKKQEKELKELERKGSKRREELLQKYSVLFLEPVYPRGLDSQVVELKERLEMELIHLGEEYHDGIRRRKEQHATEQTAKITELAREKQIAELKALKESSESNIKDIKKKLEAKRLDRIQVMMRSTSDKAAQERLKKEINNSHIQEVVQTIKLLTEKTARYQQKLEEKQAENLRAIQEKEGQLQQEAVAEYEEKLKTLTVEVQEMVKNYMKEVFPDGPE

SEQ ID NO: 492 - protein, 109 aa, synthetic construct
  REGION 1..109: note = Synthetic: 1JBG
MKYQVKQVAEISGVSIRTLHHYDNIELLNPSALTDAGYRLYSDADLERLQQILFFKEIGFRLDEIKEMLDHPNFDRKAALQSQKEILMKKKQRMDEMIQTIDRTLLSVD

SEQ ID NO: 493 - protein, 635 aa, synthetic construct
  REGION 1..635: note = Synthetic: 1JCH
MSGGDGRGHNTGAHSTSGNINGGPTGLGVGGGASDGSGWSSENNPWGGGSGSGIHWGGGSGHGNGGGNGNSGGGSGTGGNLSAVAAPVAFGFPALSTPGAGGLAVSISAGALSAAIADIMAALKGPFKFGLWGVALYGVLPSQIAKDDPNMMSKIVTSLPADDITESPVSSLPLDKATVNVNVRVVDDVKDERQNISVVSGVPMSVPVVDAKPTERPGVFTASIPGAPVLNISVNNSTPAVQTLSPGVTNNTDKDVRPAFGTQGGNTRDAVIRFPKDSGHNAVYVSVSDVLSPDQVKQRQDEENRRQQEWDATHPVEAAERNYERARAELNQANEDVARNQERQAKAVQVYNSRKSELDAANKTLADAIAEIKQFNRFAHDPMAGGHRMWQMAGLKAQRAQTDVNNKQAAFDAAAKEKSDADAALSSAMESRKKKEDKKRSAENNLNDEKNKPRKGFKDYGHDYHPAPKTENIKGLGDLKPGIPKTPKQNGGGKRKRWTGDKGRKIYEWDSQHGELEGYRASDGQHLGSFDPKTGNQLKGPDPKRNIKKYLGLKLDLTWFDKSTEDFKGEEYSKDFGDDGSVMESLGVPFKDNVNNGCFDVIAEWVPLLQPYFNHQIDISDNEYFVSFDYRDGDW

SEQ ID NO: 494 - protein, 37 aa, synthetic construct
  REGION 1..37: note = Synthetic: 1MV4
GCGKSIDDLEDELYAQKLKYKAISEELDHALKDMTSI

SEQ ID NO: 495 - protein, 97 aa, synthetic construct
  REGION 1..97: note = Synthetic: 1NO4
PLKPEEHEDILNKLLDPELAQSERTEALQQLRVNYGSFVSEYNDLTKSHEKLAAEKDDLIVSNSKLFRQIGLTEKQEEDHKKADISETITIEDLEAK

SEQ ID NO: 496 - protein, 33 aa, synthetic construct
  REGION 1..33: note = Synthetic: 1R48
CGGDNIEQKIDDIDHEIADLQAKRTRLVQQHPR

SEQ ID NO: 497 - protein, 95 aa, synthetic construct
  REGION 1..95: note = Synthetic: 1R7J
AKKKSKLEIIQAILEACKSGSPKTRIMYGANLSYALTGRYIKMLMDLEIIRQEGKQYMLTKKGEELLEDIRKFNEMRKNMDQLKEKINSVLSIRQ

SEQ ID NO: 498 - protein, 250 aa, synthetic construct
  REGION 1..250: note = Synthetic: 1QUU
GSSNEIRRLERLEHLAEKFRQKASTHETWAYGKEQILLQKDYESASLTEVRALLRKHEAFESDLAAHQDRVEQIAAIAQELNELDYHDAVNVNDRCQKICDQWDRLGTLTQKRREALERMEKLLETIDQLHLEFAKRAAPFNNWMEGAMEDLQDMFIVHSIEEIQSLITAHEQFKATLPEADGERQSIMAIQNEVEKVIQSYNIRISSSNPYSTVTMDELRTKWDKVKQLVPIRDQSLQEELARQHANER

SEQ ID NO: 499 - protein, 31 aa, synthetic construct
  REGION 1..31: note = Synthetic: 1P9I
MDQLNALLASLEAENKQLKAKVEELLAKVGE

SEQ ID NO: 500 - protein, 142 aa, synthetic construct
  REGION 1..142: note = Synthetic: 1PL5
SNTTEILTSVDVLGTHSQTGTQQSNMYTSTQKTELEIDNKDSVTECSKDMKEDGLSFVDIVLSKAASALDEKEKQLAVANEIIRSLSDEVMRNEIRITSLQGDLTFTKKCLENARSQISEKDAKINKLMEKDFQVNKEIKPY

SEQ ID NO: 501 - protein, 37 aa, synthetic construct
  REGION 1..37: note = Synthetic: 1T6F
TLYEALKENEKLHKEIEQKDNEIARLKKENKELAEVA

SEQ ID NO: 502 - protein, 96 aa, synthetic construct
  REGION 1..96: note = Synthetic: 1T3J
GSSHHHHHHSSGLVPRGSHMFTSANCSHQVQQEMATTFARLCQQVDMTQKHLEEEIARLSKEIDQLEKMQNNSKLLRNKAVQLESELENFSKQFLH

SEQ ID NO: 503 - protein, 33 aa, synthetic construct
  REGION 1..33: note = Synthetic: 1ZIL
RMKQLEDKVEELLSKQYHLENEVARLKKLVGER

SEQ ID NO: 504 - protein, 33 aa, synthetic construct
  REGION 1..33: note = Synthetic: 1ZIK
RMKQLEDKVEELLSKKYHLENEVARLKKLVGER

SEQ ID NO: 505 - protein, 34 aa, synthetic construct; identical to SEQ ID NO: 254
  REGION 1..34: note = Synthetic: 1ZII
  VARIANT 1: note = Xaa can be any naturally occurring amino acid

SEQ ID NO: 506 - protein, 58 aa, synthetic construct
  REGION 1..58: note = Synthetic: 1YSA
MKDPAALKRARNTEAARRSRARKLQRMKQLEDKVEELLSKNYHLENEVARLKKLVGER

SEQ ID NO: 507 - protein, 32 aa, synthetic construct
  REGION 1..32: note = Synthetic: 1TMZ
MDAIKKKMQMLKLDNYHLENEVARLKKLVGER

SEQ ID NO: 508 - protein, 42 aa, synthetic construct
  REGION 1..42: note = Synthetic: 1U0I
EIAALEKEIAALEKEIAALEKKIAALKEKIAALKEKIAALKE

SEQ ID NO: 509 - protein, 95 aa, synthetic construct; identical to SEQ ID NO: 497
  REGION 1..95: note = Synthetic: 1XSX

SEQ ID NO: 510 - protein, 76 aa, synthetic construct
  REGION 1..76: note = Synthetic: 1YIG
GPLGSGVGNGDDEAAELMQQVNVLKLTVEDLEKERDFYFGKLRNIELICQENEGENDPVLQRIMDILYATDEGFVI

SEQ ID NO: 511 - protein, 76 aa, synthetic construct
  REGION 1..76: note = Synthetic: 1YIB
GPLGSGVGNGDDEAAELMQQVNVLKLTVEDLEKERDFYFGKLRNIELICQENEGENDPVLQRIVDILYATDEGFVI

SEQ ID NO: 512 - protein, 476 aa, synthetic construct
  REGION 1..476: note = Synthetic: 1HCI
GSSAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHETWAYGKEQILLQKDYESASLTEVRALLRKHEAFESDLAAHQDRVEQIAAIAQELNELDYHDAVNVNDRCQKICDQWDRLGTLTQKRREALERMEKLLETIDQLHLEFAKRAAPFNNWMEGAMEDLQDMFIVHSIEEIQSLITAHEQFKATLPEADGERQSIMAIQNEVEKVIQSYNIRISSSNPYSTVTMDELRTKWDKVKQLVPIRDQSLQEELARQHANERLRRQFAAQANAIGPWIQNKMEEIARSSIQITGALEDQMNQLKQYEHNIINYKNNIDKLEGDHQLIQEALVFDNKHTNYTMEHIRVGWELLLTTIARTINEVETQILTRD

SEQ ID NO: 513 - protein, 57 aa, synthetic construct
  REGION 1..57: note = Synthetic: 1HBW
TRAHLTEVESRLERLEQLFLLIFPREDLDMILKMDSLRDIEALLTGLFVQDNVNKDA

SEQ ID NO: 514 - protein, 41 aa, synthetic construct
  REGION 1..41: note = Synthetic: 1HF9
ALKKHHENEISHHAKEIERLQKEIERHKQSIKKLKQSEDDD

SEQ ID NO: 515 - protein, 81 aa, synthetic construct
  REGION 1..81: note = Synthetic: 1IC2
MDAIKKKMQMLKLDKENALDRAEQAEADKKAAEERSKQLEDELVALQKKLKGTEDELDKYSESLKDAQEKLELADKKATDC

SEQ ID NO: 516 - protein, 157 aa, synthetic construct
  REGION 1..157: note = Synthetic: 1JY3
SACKETGWPFCSDEDWNTKCPSGCRMKGLIDEVDQDFTSRINKLRDSLFNYQKKVERKPPDADGCLHADPDLGVLCPTGCKLQDTLVRQERPIRKSIEDLRNTVDSVSRYVATRDNCCILDERFGSYCPTTCGIADFLNNYQTSVDKDLRTLEGILY

SEQ ID NO: 517 - protein, 157 aa, synthetic construct; identical to SEQ ID NO: 516
  REGION 1..157: note = Synthetic: 1JY2

SEQ ID NO: 518 - protein, 112 aa, synthetic construct
  REGION 1..112: note = Synthetic: 1L8D
MKKLLEELETKKTTIEEERNEITQRIGELKNKIGDLKTAIEELKKAKGKCPVCGRELTDEHREELLSKYHLDLNNSKNTLAKLIDRKSELERELRRIDMEIKRLTPLLTVAE

SEQ ID NO: 519 - protein, 138 aa, synthetic construct
  REGION 1..138: note = Synthetic: 1LJ2
MHSLQNVIPQQQAHIAELQVYNNKLERDLQNKIGSLTSSIEWYLRSMELDPEIKADIEQQINSIDAINPLHAFDDLESVIRNLISDYDKLFLMFKGLIQRSNYQYSFGSEAPKRERKTIRIRDPNQGGKDITEEIMSG

SEQ ID NO: 520 - protein, 61 aa, synthetic construct
  REGION 1..61: note = Synthetic: 1LR1
GSHMSEALKILNNIRTLRAQARESTLETLEEMLEKLEVVVNERREEESAAAAEVEERTRKL

SEQ ID NO: 521 - protein, 34 aa, synthetic construct [sequence omitted]

SEQ ID NO: 522 - protein, 122 aa, synthetic construct
  REGION 1..122: note = Synthetic: 1BM9
MKEEKRSSTGFLVKQRAFLKLYMITMTEQERLYGLKLLEVLRSEFKEIGFKPNHTEVYRSLHELLDDGILKQIKVKKEGAKLQEVVLYQFKDYEAAKLYKKQLKVELDRCKKLIEKALSDNF

SEQ ID NO: 523 - protein, 101 aa, synthetic construct
  REGION 1..101: note = Synthetic: 1D7M
EMANRLAGLENSLESEKVSREQLIKQKDQLNSLLASLESEGAEREKRLRELEAKLDETLKNLELEKLARMELEARLAKTEKDRAILELKLAEAIDEKSKLE

SEQ ID NO: 524 - protein, 90 aa, synthetic construct
  REGION 1..90: note = Synthetic: 1JR5
MNKNIDTVREIITVASILIKFSREDIVENRANFIAFLNEIGVTHEGRKLNQNSFRKIVSELTQEDKKTLIDEFNEGFEGVYRYLEMYTNK

SEQ ID NO: 525 - protein, 159 aa, synthetic construct
  REGION 1..159: note = Synthetic: 1JTH
ALSEIETRHSEIIKLENSIRELHDMFMDMAMLVESQGEMIDRIEYNVEHAVDYVERAVSDTKKAVKYQSKARRKKIMMAEDADMRNELEEMQRRADQLADESLESTRRMLQLVEESKDAGIRTLVMLDEQGEQLERIEEGMDQINKDMKEAEKNLTDLG

SEQ ID NO: 526 - protein, 44 aa, synthetic construct
  REGION 1..44: note = Synthetic: 1JUN
  VARIANT 1: note = Xaa can be any naturally occurring amino acid
XCGGRIARLEEKVKTLKAQNSELASTANMLREQVAQLKQKVMNY

SEQ ID NO: 527 - protein, 56 aa, synthetic construct
  REGION 1..56: note = Synthetic: 1KQL
MDKVEELLSKNYHLENEVARLKKLVDDLEDELYAQKLKYKAISEELDHALNDMTSI

SEQ ID NO: 528 - protein, 59 aa, synthetic construct
  REGION 1..59: note = Synthetic: 1GK6
RMKQLEDKVEELLSKNYHLENEVARLKKLVGDLLNVKMALDIEIATYRKLLEGEESRIS

SEQ ID NO: 529 - protein, 84 aa, synthetic construct
  REGION 1..84: note = Synthetic: 1GMJ
GSESGDNVRSSAGAVRDAGGAFGKREQAEEERYFRARAKEQLAALKKHKENEISHHAKEIERLQKEIERHKQSIKKLKQSEDDD

SEQ ID NO: 530 - protein, 349 aa, synthetic construct
  REGION 1..349: note = Synthetic: 1FXK
NVQHQLAQFQQLQQQAQAISVQKQTVEMQINETQKALEELSRAADDAEVYKSSGNILIRVAKDELTEELQEKLETLQLREKTIERQEERVMKKLQEMQVNIQEAMKGAGQNVQHQLAQFQQLQQQAQAISVQKQTVEMQINETQKALEELSRAADDAEVYKSSGNILIRVAKDELTEELQEKLETLQLREKTIERQEERVMKKLQEMQVNIQEAMKAALAEIVAQLNIYQSQVELIQQQMEAVRATISELEILEKTLSDIQGKDGSETLVPVGAGSFIKAELKDTSEVIMSVGAGVAIKKNFEDAMESIKSQKNELESTLQKMGENLRAITDIMMKLSPQAEELLAAVA

SEQ ID NO: 531 - protein, 213 aa, synthetic construct
  REGION 1..213: note = Synthetic: 1GXL
DAKEKRLREIQFEKEMIERDMREYRGFSRAVRAVFEEKERFPGLVDVVSNLIEVDEKYSLAVSVLLGGTAQNIVVRNVDTAKAIVEFLKQNEAGRVTILPLDLIDGSFNRISGLENERGFVGYAVDLVKFPSDLEVLGGFLFGNSVVVETLDDAIRMKKKYRLNTRIATLDGELISGRGAITGGREERSSNVFERRIKLKHLEQEMEETERQI

SEQ ID NO: 532 - protein, 186 aa, synthetic construct
  REGION 1..186: note = Synthetic: 1GXK
EKEMIERDMREYRGFSRAVRAVFEEKERFPGLVDVVSNLIEVDEKYSLAVSVLLGGTAQNIVVRNVDTAKAIVEFLKQNEAGRVTILPLDLIDGSFNRISGLENERGFVGYAVDLVKFPSDLEVLGGFLFGNSVVVETLDDAIRMKKKYRLNTRIATLDGELISGRGAITGGREERSSNVFERRIK

(19) United States
(12) Patent Application Publication
(10) Pub. No.: US 2024/0075120 A1
(43) Pub. Date: Mar. 7, 2024

(54) SELF-ASSEMBLING PROTEIN NANOPARTICLES ENCAPSULATING IMMUNOSTIMULATORY NUCLEID ACIDS

(71) Applicant: ALPHA-O PEPTIDES AG, Riehen (CH)

(72) Inventors: Caroline KULANGARA, Basel (CH); Sara Maria PAULILLO, Basel (CH); Matteo PIAZZA, Chester (GB); Senthil Kumar RAMAN, Basel (CH); Peter BURKHARD, Schopfheim (DE)

(21) Appl. No.: 18/297,207

(22) Filed: Apr. 7, 2023

Related U.S. Application Data
(63) Continuation of application No. 16/488,197, filed on Aug. 22, 2019, now abandoned.

(30) Foreign Application Priority Data
Feb. 23, 2017 (EP) 17157687.9

(51) Int. Cl. A61K 39/00; A61K 39/015; A61K 39/112; A61K 39/12; A61K 39/245; A61K 39/39; A61P 31/22; A61P 33/06; C12N 7/00

(52) U.S. Cl. CPC A61K 39/0013; A61K 39/015; A61K 39/0275; A61K 39/12; A61K 39/245; A61K 39/39; A61P 31/22; A61P 33/06; C12N 7/00; A61K 2039/55555; A61K 2039/55561; C07K 14/005; C12N 2710/16634

(57) ABSTRACT

The present invention relates to self-assembling protein nanoparticles encapsulating immunostimulatory nucleid acids. Furthermore, the invention relates to the use of such nanoparticles for vaccination.

### FIELD OF THE INVENTION

The present invention relates to self-assembling protein nanoparticles encapsulating immunostimulatory nucleid acids. Furthermore, the invention relates to the use of such nanoparticles for vaccination.

### BACKGROUND OF THE INVENTION

### CpGs—TLR9

Short single-stranded synthetic DNA molecules that contain a cytosine followed by a guanine are called CpG oligodeoxynucleotides (or CpG ODN). The “p” refers to the phosphodiester bond between the two consecutive nucleotides—as opposed to a CG base pairing in double stranded DNA—while some synthetic ODN have a modified phosphorothioate backbone instead to increase their in vivo stability. When the cytosine of these CpG motifs is unmethylated, they may act as immunostimulatory molecules. Due to their abundance in microbial genomes in contrast to their relative rarity in the genomes of vertebrates—in mammals about 70% to 80% of the cytosines in all CpG pairs are methylated—CpG motifs are considered pathogen-associated molecular patterns (PAMPs). The CpG PAMP is recognized by the Toll-Like Receptor 9 (TLR9), which is a so-called pattern recognition receptor. TLR9 is the toll like receptor that recognizes DNA both from bacteria and viruses, while TLR3, TLR7 and TLR8 recognize pathogen-derived RNA. TLR9 is constitutively expressed only in plasmacytoid dendritic cells and B cells in higher primates and humans, thus unmethylated CpG dinucleotide sites can be detected by TLR9 on these cells in humans. This is used by the immune system to detect intracellular infection.

### RNA

Pathogen-derived RNA is also recognized by toll like receptors. TLR3 recognizes double-stranded RNA and poly I:C, largely from viruses that carry a genome of double-stranded RNA; TLR7 recognizes single-stranded RNA from RNA viruses while TLR8 recognizes small synthetic compounds, single-stranded viral RNA and phagocytized bacterial RNA.

### TLR3

The most commonly used experimental TLR3 agonist is polyl:polyC (pIC). pIC is a large synthetic polymeric complex mimicking double-stranded RNA (dsRNA). Preparations of pIC vary in the distribution of the strand length, the solubility, and other biological properties including toxicity.

Experimental studies have shown that TLR3 can trigger apoptosis in cancer cells. In addition, there are other dsRNA binding receptors in cytoplasm such as MDA5 and RIG-I, which can also bind pIC and contribute to apoptosis in cancer cells. The capability of TLR3 to induce apoptosis and activate the immune system at the same time renders TLR3 ligands such as pIC an attractive therapeutic option for cancer treatment.

### TLR7 and TLR8

Localized in the endosomes TLR7 and TLR8 recognize single-stranded RNA (ssRNA). This is a common feature of the genomes of ssRNA viruses such as Influenza, Sendai, and Coxsackie B viruses that are internalized by immune cells such as macrophages or dendritic cells. While TLR7 can recognize GU-rich ssRNA the presence of GU-rich sequences in ssRNA is not sufficient to stimulate TLR7. Imiquimod is a prescription medication that acts as an immune response modifier by interacting with TLR7. Imiquimod is used to treat superficial basal cell carcinoma, genital warts, and actinic keratosis. Resiquimod (R-848) and Gardiquimod are derivatives of Imiquimod.

### SUMMARY OF THE INVENTION

In a first aspect the invention relates to a composition for inducing an immune response in a subject comprising:
(a) A self-assembling protein nanoparticle (SAPN) consisting of a multitude of building blocks of formula (I)

X1-ND1-L1-ND2-Y1 (I),
consisting of a continuous chain comprising a coiled-coil oligomerization domain ND1, a linker L1, a coiled-coil oligomerization domain ND2 and further substituents X1 and Y1, wherein
ND1 is a coiled-coil oligomerization domain that comprises oligomers (ND1)ₘ of m subunits ND1,
ND2 is a coiled-coil oligomerization domain that comprises oligomers (ND2)ₙ of n subunits ND2,
m and n each is a figure between 2 and 10, with the proviso that m is not equal n and not a multiple of n, and n is not a multiple of m,
L1 is a peptide linker with an overall positive charge of at least +2 at physiological conditions,
X1 is absent or a peptide or protein sequence comprising 1 to 1000 amino acids that may be further substituted.
Y1 is absent or a peptide or protein sequence comprising 1 to 1000 amino acids that may be further substituted,
wherein the multitude of building blocks of formula (I) is optionally co-assembled with a multitude of building blocks of formula (II)

X2-ND3-L2-ND4-Y2 (II),
consisting of a continuous chain comprising a coiled-coil oligomerization domain ND3, a linker L2, a coiled-coil oligomerization domain ND4, and further substituents X2 and Y2, wherein
ND3 is a coiled-coil oligomerization domain that comprises oligomers (ND3)_{y} of y subunits ND3,
ND4 is a coiled-coil oligomerization domain that comprises oligomers (ND4)_{z} of z subunits ND4,
y and z each is a figure between 2 and 10, with the proviso that y is not equal z and not a multiple of z, and z is not a multiple of y, and wherein
either ND3 is identical to ND1, or ND4 is identical to ND2 or both ND3 and ND4 are identical to ND1 and ND2, respectively,
L2 is a peptide linker with an overall positive charge of at least +2 at physiological conditions,
X2 is absent or a peptide or protein sequence comprising 1 to 1000 amino acids that may be further substituted
Y2 is absent or a peptide or protein sequence comprising 1 to 1000 amino acids that may be further substituted,
(b) an immunostimulatory substance, wherein said immunostimulatory substance is a nucleic acid derivative wherein said nucleic acid derivative is encapsulated into said SAPN.

In a second aspect the invention relates to a method of vaccinating a human or non-human animal, which comprises administering an effective amount of a composition as described herein to a subject in need of such vaccination.

In a third aspect the invention relates to a method of producing a SAPN as described herein, comprising i) adding a SAPN to a buffer comprising a nucleic acid derivative and ii) refolding the SAPN in the presence of the nucleic acid derivative using a regular refolding protocol.

### BRIEF DESCRIPTION OF THE FIGURES

FIG. 1: Schematic diagram of a monomer of an encapsulating CpG nanoparticle.

The following are the building blocks of the monomer:
X1 is a peptide or protein sequence comprising 1 to 1000 amino acids that may be further substituted.
ND1 is a coiled coil that forms oligomers (ND1)ₘ of m subunits ND1
L1 is a peptide linker with an overall positive charge of +3,
ND2 is a coiled coil that forms oligomers (ND2)ₙ of n subunits ND2
Y1 is absent or a peptide or protein sequence comprising 1 to 1000 amino acids that may be further substituted.

FIG. 2: Molecular model of DEDDLI-RR.

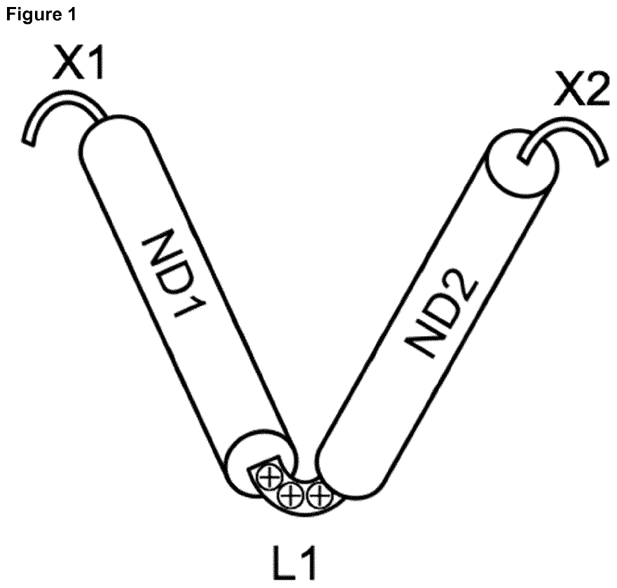

A) X-ray crystal structures of the TLR5 and TLR9 receptors with their respective agonists: The TLR5-dimer interacts with two molecules of flagellin (yellow and magenta), while the TLR9 interacts with CpG. B) Left: Monomeric building block of the self-assembling protein composed of the his-tag (X1) pentameric coiled coil (ND1), the dimeric coiled-coil (ND2) and the D0 and D1 domains of flagellin (X2). The two coiled-coil oligomerization domains ND1 and ND2 are joined by a linker with three positive charges (L1). Right: CpG molecule. C) Assembled protein nanoparticle with 60 protein chains and about 36 CpG molecules encapsulated in the central cavity. For better clarity the protein chains inside the circle (representing positive charges) are not shown to make the (negatively charged) CpG molecules inside the particle visible. Note, not all structures in panels A), B) and C) are drawn to size.

FIG. 3: Vector map of pPEP-T.

“prom”: promoter; “term”: terminator; “ori”: origin; “bp”: base pairs; “amp”: ampicillin resistance gene.

FIG. 4: SDS-PAGE of the construct DEDDLI-RR.

This construct has a theoretical molecular weight of 44.8 kDa

A) Expression levels with two different concentrations for the sample

UI—Uninduced

I—Induced

B) Elution profile from the FPLC. The protein elutes at 120 to 122 mM imidazole.

C) Purity after Ni-affinity purification. First lane: Mw Marker; CL: cleared lysate; lanes 3 to 9: flow through; lanes 15 to 20: elution peak.

D) Mass-spec analysis before (bottom) and after (top) coupling of NHS-nicotine to DEDDLI-RR.

FIG. 5: Relative Fluorescence Units (RFUs) with and without encapsulation of fluorescent-labelled ODN1826F in construct DEDDLI-RR.

RFU values for the CpG-ODN1826F only (black columns) and encapsulated CpG-ODN1826F in the SAPN DEDDLI-RR (dashed columns) for increasing encapsulation ratios. The molar ratios of protein chains of DEDDLI-RR to DNA chains of ODN1826F are indicated.

FIG. 6: Difference in Relative Fluorescence Units (RFUs) after encapsulation of fluorescent-labelled ODN1826F in construct DEDDLI-RR.

RFU values for the CpG-ODN1826F only (black diamonds) and difference corresponding to the free CpG in the sample of the encapsulated CpG in DEDDLI-RR (dashed squares) for increasing encapsulation ratios. The two curves are closely overlapping.
The values of the difference in the RFU are calculated as the signal from DEDDLI-RR with encapsulated CpG at a given CpG encapsulation ratio minus the signal at the encapsulation ratio of 1:0.6.
The values of the ratios of the “difference” curve (dashed squares) are calculated as the ratio minus 0.6.

FIG. 7: Transmission electron micrograph of DEDDLI-RR.

After refolding and co-assembly of recombinantly expressed protein, the sample was adsorbed on carbon-coated grids and negatively stained with 2% uranyl acetate. The nanoparticles have the sequence SEQ ID NO:1 described in Example 1. The bars for the top and bottom sections represent 200 nm and 500 nm, respectively.

FIG. 8: Immune response for DEDDLI-RR with and without encapsulated ODN1826.

Three injection modes (IM, IN and IV) at two protein concentrations of 10 μg and 30 μg each with their corresponding antibody titers. 0.85 μg and 2.56 μg of CpG were encapsulated for the 10 μg and 30 μg doses, respectively indicated by “+” or “−” signs. The antibody titer was determined by an ELISA binding assay to a plate coated with BSA-nicotine, i.e. nicotine covalently coupled to BSA. Significant increases in antibody titers can be observed in the samples from encapsulated CpG in the immunization.

FIG. 9: Relative Fluorescence Units (RFUs) with and without encapsulation of fluorescent-labelled ODN1826F in the constructs DEDDLI-RR, 2RR and 3RR.

RFU values for the CpG-ODN1826F only (diamonds) and encapsulated CpG-ODN1826F in the SAPN DEDDLI-RR (squares), 2RR (triangles) and 3RR (circles) for increasing encapsulation ratios. The molar ratios of protein chains of DEDDLI-RR to DNA chains of ODN1826F are indicated.

♦ CpG only (i.e. without encapsulation)

▪ DEDDLI-RR

▴ 2RR

● 3RR

FIG. 10: Immune response for LIVELI-based constructs with and without encapsulated ODN1826.

Groups of five Balb/C mice each were immunized with a dose of 30 μg protein, either with (LIVELI1-RR and LIVELI2-RR) or without encapsulated CpG (LIVELI1 and LIVELI2). The amount of encapsulated CpG in the LIVELI1-RR and LIVELI2-RR doses is about 2.5 μg. Three injections each two weeks apart were given intramuscular. Significant increases in antibody titers can be observed in the samples from encapsulated CpG.

FIG. 11: Transmission electron micrograph of LIVELI1, LIVELI2, LIVELI1-RR and LIVELI2-RR.

After refolding and co-assembly of recombinantly expressed protein, the samples were adsorbed on carbon-coated grids and negatively stained with 2% uranyl acetate. The nanoparticles correspond to A) LIVELI1, B) LIVELI2, C) LIVELI1-RR and D) LIVELI2-RR and have the sequence SEQ ID NO:20, SEQ ID NO:21, SEQ ID NO:18 and SEQ ID NO:19, respectively, described in Example 10. The bars in all panels represent 200 nm.

FIG. 12: Relative Fluorescence Units (RFUs) with and without encapsulation of fluorescent-labelled ODN1826F in construct CC-RR.

RFU values for the CpG-ODN1826F only (black columns) and encapsulated CpG-ODN1826F in the SAPN CC-RR (dashed columns) for increasing encapsulation ratios. The molar ratios of protein chains of DEDDLI-RR to DNA chains of ODN1826F are indicated.

FIG. 13: Molecular model of CC-RR-NN.

A) Monomeric building block of the first self-assembling protein chain composed of the his-tag and CeITOS (X1) the first coiled-coil domain (ND1), the second coiled-coil domain (ND2) and the second molecule of CeITOS (Y1) in which the two coiled-coil domains are joined by a short peptide linker with three positive charges (L1). B) Monomeric building block of the second self-assembling protein chain composed of the his-tag and CeITOS (X2) the first coiled-coil domain (ND3), the second coiled-coil domain (ND4) and the D0 and D1 domains of flagellin (Y2), in which the two coiled-coil domains are joined by a short peptide linker with three positive charges (L2). C) A CpG molecule (not drawn to size with panels A and B). During refolding co-assembly and encapsulation occur at the same time. D) Assembled protein nanoparticle with 60 protein chains at a co-assembly ratio of 58:2 of the first and second protein chains and about 36 CpG molecules encapsulated in the central cavity. For better clarity the protein chains inside the circle (representing positive charges) are not shown to make the (negatively charged) CpG molecules inside the particle visible. E) Transmission electron micrograph of the co-assembled SAPNs with encapsulated CpG. The bar represents 100 nm.

FIG. 14: Transmission electron micrograph of RR-SSIEF.

After refolding of recombinantly expressed protein, the sample was adsorbed on carbon-coated grids and negatively stained with 2% uranyl acetate. The nanoparticles correspond to RR-SSIEF and have the sequence SEQ ID NO:34 described in Example 12 with encapsulated CpG ODN1585 (SEQ ID NO:39). The bar represents 200 nm.

### DETAILED DESCRIPTION OF THE INVENTION

In the present invention DNA and/or RNA binding sites are described that are built-in into the architecture of SAPNs with the goal to encapsulate nucleic acids into the SAPN. The SAPNs are described e.g. in Raman S. K. et al. Nanomed 2006, 2(2): 95-102; Pimentel T. A., et al. Chem Biol Drug Des. 2009. 73(1): 53-61; Indelicato, G., et al. Biophys J. 2016, 110(3): 646-660; Karch, C. P., et al. Nanomedicine 2016, 13(1): 241-251. The SAPNs are also described in WO2004071493, WO2009109428 and WO2015104352. In a first aspect the invention relates to a composition for inducing an immune response in a subject comprising:
(a) A self-assembling protein nanoparticle (SAPN) consisting of a multitude of building blocks of formula (I)

X1-ND1-L1-ND2-Y1 (I),
consisting of a continuous chain comprising a coiled-coil oligomerization domain ND1, a linker L1, a coiled-coil oligomerization domain ND2 and further substituents X1 and Y1, wherein
ND1 is a coiled-coil oligomerization domain that comprises oligomers (ND1)ₘ of m subunits ND1,
ND2 is a coiled-coil oligomerization domain that comprises oligomers (ND2)ₙ of n subunits ND2,
m and n each is a figure between 2 and 10, with the proviso that m is not equal n and not a multiple of n, and n is not a multiple of m,
L1 is a peptide linker with an overall positive charge of at least +2 at physiological conditions,
X1 is absent or a peptide or protein sequence comprising 1 to 1000 amino acids that may be further substituted.
Y1 is absent or a peptide or protein sequence comprising 1 to 1000 amino acids that may be further substituted,
wherein the multitude of building blocks of formula (I) is optionally co-assembled with a multitude of building blocks of formula (II)

X2-ND3-L2-ND4-Y2 (II),
consisting of a continuous chain comprising a coiled-coil oligomerization domain ND3, a linker L2, a coiled-coil oligomerization domain ND4, and further substituents X2 and Y2, wherein
ND3 is a coiled-coil oligomerization domain that comprises oligomers (ND3)_{y} of y subunits ND3,
ND4 is a coiled-coil oligomerization domain that comprises oligomers (ND4)_{z} of z subunits ND4,
y and z each is a figure between 2 and 10, with the proviso that y is not equal z and not a multiple of z, and z is not a multiple of y, and wherein
either ND3 is identical to ND1, or ND4 is identical to ND2 or both ND3 and ND4 are identical to ND1 and ND2, respectively,
L2 is a peptide linker with an overall positive charge of at least +2 at physiological conditions,
X2 is absent or a peptide or protein sequence comprising 1 to 1000 amino acids that may be further substituted
Y2 is absent or a peptide or protein sequence comprising 1 to 1000 amino acids that may be further substituted,
(b) an immunostimulatory substance, wherein said immunostimulatory substance is a nucleic acid derivative wherein said nucleic acid derivative is encapsulated into said SAPN.

It has now surprisingly been found that if the linker connecting the two oligomerization domains of the SAPN contains a stretch of positively charged amino acids, thus rendering the overall charge of the linker to at least plus two, negatively charged nucleic acids can be encapsulated into the SAPN. This is because the linker harboring the positive charges is conveniently oriented towards the central cavity of the SAPN thus providing a positively charged surface coating of the central cavity, akin of the positively charged cavities of viral capsids that encapsulate the genomic material of the virus. This was nevertheless unexpected as in a SAPN with T1 icosahedral symmetry 60 protein chains assemble to for the SAPN, thus with at least two positive charges per linker as many as 120 positive charges will be lining up the relatively small space of the central cavity thus leading to significant repulsive forces that counteract formation of SAPNs during refolding.

It is noteworthy, that this encapsulation of nucleic acids in SAPNs does not need any special chemical attachment of the nucleic acids to the SAPNs. Encapsulation of the nucleic acids occurs when adding the nucleic acid to the refolding buffer before refolding and then refolding the SAPNs in the presence of nucleic acids using the regular refolding protocol.

Specific nucleic acids that can be encapsulated into the SAPN may contain immunostimulatory properties. For example, using SAPNs with encapsulated CpG during an immunization protocol increases the overall immune response significantly. The SAPNs of the present invention therefore offer an elegant way to efficiently increase the immune response and hence the immunogenicity of SAPN-based vaccines.

### Monomeric Building Blocks

A peptide (or polypeptide or protein) is a chain or sequence of amino acids covalently linked by amide bonds. The peptide may be natural, modified natural, partially synthetic or fully synthetic. Modified natural, partially synthetic or fully synthetic is understood as meaning not occurring in nature. The term amino acid embraces both naturally occurring amino acids selected from the 20 essential natural α-L-amino acids, synthetic amino acids, such as α-D-amino acids, 6-aminohexanoic acid, norleucine, homocysteine, or the like, as well as naturally occurring amino acids which have been modified in some way to alter certain properties such as charge, such as phoshoserine or phosphotyrosine, or other modifications such as n-octanoyl-serine, or the like. Derivatives of amino acids are amino acids in which for example the amino group forming the amide bond is alkylated, or a side chain amino-, hydroxyl- or thio-group is alkylated or acylated, or a side chain carboxy-group is amidated or esterified. Preferably a peptide or protein of the invention comprises amino acids selected from the 20 essential natural α-L-amino acids.

In a rough approximation, peptides can be distinguished from proteins on the basis of their size, i.e. approximately a chain of 50 amino acids or less can be considered to be a peptide, while longer chains can be considered to be proteins. Thus, the term “peptide” as used herein refers to an amino acid chain of 50 amino acids or less, preferably to an amino acid chain of 2 to 50 amino acids, the term “protein” as used herein refers to an amino acid chain of more than 50 amino acids, preferably to an amino acid chain of 51 to 10000 amino acids. Dipeptides are the shortest peptides and consist of 2 amino acids joined by a single peptide bond. Likewise, tripeptides consist of three amino acids, tetrapeptides consist of four amino acids, etc. A polypeptide is a long, continuous, and unbranched peptide chain. In the literature boundaries of the size that distinguish peptides from proteins are somewhat weak. Sometimes long “peptides” such as amyloid beta have been considered proteins, and vice versa smaller proteins such as insulin have been referred to as peptides.

Oligomerization domains according to the invention are coiled-coils. A coiled coil is a protein sequence with a contiguous pattern of mainly hydrophobic residues spaced 3 and 4 residues apart, which assembles to form a multimeric bundle of helices, as will be explained in more detail herein below.

The components ND1, ND2, X1 and Y1 of the monomeric building block of formula (I) and/or the components (ND3, ND4, X2 and Y2) of the monomeric building block of formula (II) may optionally be further substituted by targeting entities, or substituents reinforcing the adjuvant properties of the nanoparticle. Substituted means a replacement of one chemical group on the monomeric building block by another chemical group yielding a substituent that is covalently linked to the monomeric building block. Such substituents may be an immunostimulatory nucleic acid, preferably an oligodeoxynucleotide containing deoxyinosine, an oligodeoxynucleotide containing deoxyuridine, an oligodeoxynucleotide containing a CG motif, CpGs, imiquimod, resiquimod, gardiquimod, an inosine and cytidine containing nucleic acid molecule, or the like. A particular targeting entity considered as substituent is an ER-targeting signal, i.e. a signal peptide that induces the transport of a protein or peptide to the endoplasmic reticulum (ER).

In a preferred embodiment, the building blocks of formula (I) or (II) comprises either substituent X1 or substituent Y1 or substituent X2 or substituent Y2.

In another preferred embodiment, the building blocks of formula (I) or (II) comprises substituents X1 and Y1 or substituents X2 and Y2. Thus in a most preferred embodiment the substituent X1, X2, Y1 or Y2 is a peptide or protein substituent representing an extension of the protein chain, e.g. as X1-ND1-L1-ND2-Y1 or X2-ND3-L2-ND4-Y2 usually at one end, preferably at both ends to generate a combined single continuous protein sequence. Conveniently, such a single continuous protein chain may be expressed in a recombinant protein expression system as one single molecule. Substituents X1, Y1, X2 and Y2 independently form each other are a peptide or a protein sequence comprising 1 to 1000 amino acids preferably sequences corresponding to fully folded proteins or protein domains to be used either as B-cell epitopes, or flagellin or a subset of its four domains as described in WO2015104352 to enhance the immune response.

Flagellin has a molecular architecture that is composed of four domains D0, D1, D2 and D3. The protein chain starts with the N-terminus in the D0 domain and runs in a big loop through the other domains D1, D2 and D3 to the tip of the molecule where it turns and runs back through D3, D2 and D1 to bring its C-terminal end in the D0 domain very close to the N-terminal end. Flagellin has two modes of activation of the innate immune system. The first mode is by binding to the TLRS receptor mainly through a highly conserved portion of its D1 domain (Yoon et al., loc. cit.). The other mode of activation is by interaction with the inflammasome mainly through a highly conserved C-terminal portion of its D0 domain (Lightfield K. L. et al., Nat Immunol. 2008, 9:1171-8).

Thus in a preferred embodiment at least one of substituents X1, Y1, X2 and Y2 is a full length flagellin e.g. a full length Salmonella typhimurium flagellin or a flagellin comprising only two or three domains, preferably a flagellin comprising at least the TLR5 binding domain D1 more preferably a flagellin comprising the D0 and D1 domains, in particular the flaggellin as shown in SEQ ID NO: 6. The missing domain(s) may be substituted by a flexible linker segment of 1 to 20 amino acids joining the two ends of the remaining flagellin sequence, or they may be replaced by a fully folded protein antigen. In a preferred embodiment the flexible linker comprises the amino acid sequence as shown in SEQ ID NO: 9. The flexible linker region may contain suitable attachment sites for the covalent coupling of antigens. Thus, a flagellin derivative construct lacking the D2 and D3 domains of flagellin can easily be engineered, simply by connecting the protein chain at the interface of the D1 and D2 domains. Similar, the tip domains (either D3, or D2 and D3 together) can be replaced by a protein antigen, provided this protein antigen with its N- and C-termini can be connected to the N- and C-termini at the interface between D1 and D2. The tip domains D2 and D3 can also be replaced by a peptide sequence with suitable residues for the covalent coupling of antigen molecules.

In another preferred embodiment X1, Y1, X2 and Y2 independently from each other may also comprise a string of one or more CD4 or CD8 epitopes. In another preferred embodiment X1, Y1, X2 and Y2 independently from each other may comprise a combination of one or more of these types of immunological relevant peptide and protein sequences.

A tendency to form oligomers means that such proteins can form oligomers depending on the conditions, e.g. under denaturing conditions they are monomers, while under physiological conditions they may form, for example, dimers, trimers, tetramers or pentamers. Under predefined conditions they adopt one single oligomerization state, which is needed for nanoparticle formation. However, their oligomerization state may be changed upon changing conditions, e.g. from trimers to dimers upon decreasing salt concentration (Burkhard P. et al., Protein Science 2000, 9:2294-2301) or from pentamers to monomers upon decreasing pH.

A building block architecture according to formula (I) or (II) is clearly distinct from viral capsid proteins. Viral capsids are composed of either one single protein, which forms oligomers of 60 or a multiple thereof, as e.g. the hepatitis virus B particles (EP 1 262 555, EP 0 201 416), or of more than one protein, which co-assemble to form the viral capsid structure, which can adopt also other geometries apart from icosahedra, depending on the type of virus (Fender P. et al., Nature Biotechnology 1997, 15:52-56). SAPNs of the present invention are also clearly distinct from virus-like particles, as they (a) are constructed from other than viral capsid proteins and (b) that the cavity in the middle of the nanoparticle is too small to accommodate the DNA/RNA of a whole viral genome.

Protein oligomerization domains are well-known (Burkhard P. et al., Trends Cell Biol 2001, 11:82-88). In the present invention the oligomerization domains are a coiled-coil domain. A coiled coil is a protein sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled coils with sequences including some irregular distribution of the 3 and 4 residues spacing are also contemplated. Hydrophobic residues are in particular the hydrophobic amino acids Val, Ile, Leu, Met, Tyr, Phe and Trp. Mainly hydrophobic means that at least 50% of the residues must be selected from the mentioned hydrophobic amino acids.

### Heptad Repeats and Coiled Coils

For example, in a preferred monomeric building block of formula (I) and/or (II), ND1, ND2, ND3 and/or ND4 comprise a heptad repeat or an undecad repeat, more preferably a heptad repeat, in particular proteins of any of the formulae

[aa(a)-aa(b)-aa(c)-aa(d)-aa(e)-aa(f)-aa(g)]ₓ (IIIa),

[aa(b)-aa(c)-aa(d)-aa(e)-aa(f)-aa(g)-aa(a)]ₓ (IIIb),

[aa(c)-aa(d)-aa(e)-aa(f)-aa(g)-aa(a)-aa(b)]ₓ (IIIc),

[aa(d)-aa(e)-aa(f)-aa(g)-aa(a)-aa(b)-aa(c)]ₓ (IIId),

[aa(e)-aa(f)-aa(g)-aa(a)-aa(b)-aa(c)-aa(d)]ₓ (IIIe),

[aa(f)-aa(g)-aa(a)-aa(b)-aa(c)-aa(d)-aa(e)]ₓ (IIIf),

[aa(g)-aa(a)-aa(b)-aa(c)-aa(d)-aa(e)-aa(f)]ₓ (IIIg),

wherein aa means an amino acid or a derivative thereof, aa(a), aa(b), aa(c), aa(d), aa(e), aa(f), and aa(g) are the same or different amino acids or derivatives thereof, preferably aa(a) and aa(d) are the same or different hydrophobic amino acids or derivatives thereof; and x is a figure between 2 and 20, preferably between 3 and 10.

A heptad is a heptapeptide of the formula aa(a)-aa(b)-aa(c)-aa(d)-aa(e)-aa(f)-aa(g) (IIIa) or any of its permutations of formulae (IIIb) to (IIIg).

Preferred are monomeric building blocks of formula (I) or (II) wherein the protein oligomerization domain ND1, ND2, ND3 and/or ND4 comprise
(1) a protein of any of the formulae (IIIa) to (IIIg) wherein x is 3, and aa(a) and aa(d) are selected from the 20 natural α-L-amino acids such that the sum of scores from Table 1 for these 6 amino acids is at least 14, and such proteins comprising up to 17 further heptads; or
(2) a protein of any of the formulae (IIIa) to (IIIg) wherein x is 3, and aa(a) and aa(d) are selected from the 20 natural α-L-amino acids such that the sum of scores from Table 1 for these 6 amino acids is at least 12, with the proviso that one amino acid aa(a) is a charged amino acid able to form an inter-helical salt bridge to an amino acid aa(d) or aa(g) of a neighboring heptad, or that one amino acid aa(d) is a charged amino acid able to form an inter-helical salt bridge to an amino acid aa(a) or aa(e) of a neighboring heptad, and such proteins comprising up to two further heptads. A charged amino acid able to form an inter-helical salt bridge to an amino acid of a neighboring heptad is, for example, Asp or Glu if the other amino acid is Lys, Arg or His, or vice versa.

TABLE 1
Scores of amino acid for determination of preference
(coiled−coil propensity)

| Amino acid | Position aa(a) | Position aa(d) |
|---|---|---|
| L (Leu) | 3.5 | 3.8 |
| M (Met) | 3.4 | 3.2 |
| I (Ile) | 3.9 | 3.0 |
| Y (Tyr) | 2.1 | 1.4 |
| F (Phe) | 3.0 | 1.2 |
| V (Val) | 4.1 | 1.1 |
| Q (Gln) | −0.1 | 0.5 |
| A (Ala) | 0.0 | 0.0 |
| W (Trp) | 0.8 | −0.1 |
| N (Asn) | 0.9 | −0.6 |
| H (His) | −1.2 | −0.8 |
| T (Thr) | 0.2 | −1.2 |
| K (Lys) | −0.4 | −1.8 |
| S (Ser) | −1.3 | −1.8 |
| D (Asp) | −2.5 | −1.8 |
| E (Glu) | −2.0 | −2.7 |
| R (Arg) | −0.8 | −2.9 |
| G (Gly) | −2.5 | −3.6 |
| P (Pro) | −3.0 | −3.0 |
| C (Cys) | 0.2 | −1.2 |

Also preferred are monomeric building blocks of formula (I) or (II) wherein the protein oligomerization domain ND1, ND2, ND3 and/or ND4 comprise a protein selected from the following preferred proteins:
(11) Protein of any of the formulae (IIIa) to (IIIg) wherein
aa(a) is selected from Val, Ile, Leu and Met, and a derivative thereof, and
aa(d) is selected from Leu, Met, Val and Ile, and a derivative thereof.
(12) Protein of any of the formulae (IIIa) to (IIIg) wherein one aa(a) is Asn and the other aa(a) are selected from Asn, Ile and Leu, and aa(d) is Leu. Such a protein is usually a dimerization domain.
(13) Protein of any of the formulae (IIIa) to (IIIg) wherein aa(a) and aa(d) are both Leu or both Ile. Such a protein is usually a trimerization domain.
(14) Protein of any of the formulae (IIIa) to (IIIg) wherein aa(a) and aa(d) are both Trp. Such a protein is usually a pentamerization domain.
(15) Protein of any of the formulae (IIIa) to (IIIg) wherein aa(a) and aa(d) are both Phe. Such a protein is usually a tetramerization domain.
(16) Protein of any of the formulae (IIIa) to (IIIg) wherein aa(a) and aa(d) are both either Trp or Phe. Such a protein is usually a pentamerization domain.
(17) Protein of any of the formulae (IIIa) to (IIIg) wherein aa(a) is either Leu or Ile, and one aa(d) is Gln and the other aa(d) are selected from Gln, Leu and Met. Such a protein has the potential to be a pentamerization domain.

Other preferred proteins are proteins (1), (2), (11), (12), (13), (14), (15) (16) and (17) as defined hereinbefore, and wherein further
(18) at least one aa(g) is selected from Asp and Glu and aa(e) in a following heptad is Lys, Arg or His; and/or
(19) at least one aa(g) is selected from Lys, Arg and His, and aa(e) in a following heptad is Asp or Glu, and/or
(20) at least one aa(a to g) is selected from Lys, Arg and His, and an aa(a to g) 3 or 4 amino acids apart in the sequence is Asp or Glu. Such pairs of amino acids aa(a to g) are, for example aa(b) and aa(e) or aa(f).

Coiled-coil prediction programs such as PCOILS (http://toolkit.tuebingen.mpg.de/pcoils; Gruber M. et al., J. Struct. Biol. 2006, 155(2): 140-5) or MULTICOIL (http://groups.csail.mitedu/cb/multicoil/cgi-bin/multicoil.cgi) can predict coiled-coil forming protein sequences. Therefore, in a monomeric building block of formula (I) or (II) ND1, ND2, ND3 and/or ND4 comprise a protein that contain at least a sequence two heptad-repeats long that is predicted by the coiled-coil prediction program PCOILS to form a coiled-coil with higher probability than 0.9 for all its amino acids with at least one of the window sizes of 14, 21, or 28.

In a more preferred monomeric building block of formula (I) or (II) ND1, ND2, ND3 and/or ND4 comprises a protein that contains at least one sequence three heptad-repeats long that is predicted by the coiled-coil prediction program PCOILS to form a coiled-coil with higher probability than 0.9 for all its amino acids with at least one of the window sizes of 14, 21, or 28.

In another more preferred monomeric building block of formula (I) or (II) ND1, ND2, ND3 and/or ND4 comprises a protein that contains at least two separate sequences two heptad-repeats long that are predicted by the coiled-coil prediction program PCOILS to form a coiled-coil with higher probability than 0.9 for all its amino acids with at least one of the window sizes of 14, 21, or 28.

### The RCSB Structural Database

Known coiled-coil sequences may be retrieved from data banks such as the RCSB protein data bank (http://www.rcsb.org).

### Pentameric Coiled Coils

Pentameric coiled coils can be retrieved from the RCSB database (http://www.rcsb.org/pdb/) by the search for the symmetry in biological assembly using the discriminator “Protein symmetry is cyclic—C5” combined with a text search for “coiled” or “zipper”. A list of suitable entries contains 4PN8, 4PND, 4WBA, 3V2N, 3V2P, 3V2Q, 3V2R, 4EEB, 4EED, 3MIW, 1MZ9, 1FBM, 1VDF, 2GUV, 2HYN, 1ZLL, 1T8Z.

### Tetrameric, Trimeric and Dimeric Coiled Coils

Likewise, tetrameric coiled coils can be retrieved using “Protein symmetry is ‘cyclic—C4’”, trimeric coiled coils can be retrieved using “Protein symmetry is ‘cyclic—C3’” and dimeric coiled coils using “Protein symmetry is ‘cyclic—C2’”, each combined with a text search for “coiled” or “zipper”.

For tetrameric coiled coils this yields the following suitable entries: 5D60, 5D5Y, 5AL6, 4WB4, 4BHV, 4C5Q, 4GJW, 4H7R, 4H8F, 4BXT, 4LTO, 4LTP, 4LTQ, 4LTR, 3ZDO, 3RQA, 3R4A, 3R4H, 3TSI, 3K4T, 3F6N, 2O6N, 2OVC, 2O1J, 2O1K, 2AG3, 2CCE, 1YBK, 1U9F, 1U9G, 1U9H, 1USD, 1USE, 1UNT, 1UNU, 1UNV, 1UNW, 1UNX, 1UNY, 1UNZ, 1UO0, 1UO1, 1UO2, 1UO3, 1UO4, 1UO5, 1W5I, 1W5L, 1FE6, 1G1I, 1G1J, 1EZJ, 1RH4, 1GCL.

For trimeric coiled coils this yields the following suitable entries: 5TOH, 5TO1, 5K92, 5KB0, 5KB1, 5KB2, 5KKV, 5EFM, 2N64, 5ABS, 5IEA, 5APP, 5APQ, 5APS, 5APY, 5APZ, 5D5Z, 4YPC, 4YV3, 4CGB, 4CGC, 4CJD, 4R0R, 4UW0, 4P67, 4OXM, 3W8V, 3W92, 3W93, 4I2L, 4K8U, 4JBZ, 3VTQ, 4L1R, 4JDO, 4J4A, 4E52, 3VY1, 3ZMF, 3VU5, 3VU6, 2YNY, 2YNZ, 2YO0, 2YO1, 2YO2, 4G1A, 4GIF, 3TQ2, 4DZK, 4DZL, 4DZN, 3TE3, 3R48, 3SWF, 3SWY, 3PR7, 2YKO, 2YKP, 2YKQ, 3NTN, 3PP5, 3MKO, 3MGN, 3NWA, 3NWD, 3NWF, 3L35, 3L36, 3L37, 3M9B, 3M9D, 2X6P, 3LJM, 3AHA, 3H7X, 3H7Z, 3LT6, 3LT7, 3GJP, 2KP8, 3KPE, 2WPR, 2WPS, 2WPY, 2WPZ, 2WQ0, 2WQ1, 2WQ2, 2WQ3, 3HFC, 3HFE, 3HRN, 3HRO, 3H5F, 3H5G, 2WG5, 2WG6, 2W6B, 2JJL, 2VRS, 3EFG, 3DUZ, 2OT5, 2Z2T, 2QIH, 3BK6, 2O7H, 2R32, 2JGO, 2Q7C, 2Q3I, 2Q5U, 2IBL, 1ZV8, 1ZVB, 2FXP, 1WT6, 2AKF, 1TGG, 1SLQ, 1S9Z, 1PW9, 1PWB, 1M7L, 1GZL, 1KYC, 1KFM, 1KFN, 1IJ0, 1IJ1, 1IJ2, 1IJ3, 1HQJ, 1QU1, 1B08, 1CZQ, 1CUN, 1SVF, 1CE0, 1PIQ, 1AQ5, 1AVY, 1HTN, 1AA0, 1ZIJ, 1ZIM, 1CIO, 1SWI, 1GCM, 1HUP

For dimeric coiled coils this yields the following suitable entries: 5M97, 5M9E, 5FIY, 5F4Y, 5D3A, 5HMO, 5EYA, 5IX1, 5IX2, 5JHF, 5JVM, 5JVP, 5JVR, 5JVS, 5JVU, 5JX1, 5FCN, 5HHE, 2N9B, 4ZRY, 4Z6Y, 4YTO, 4ZI3, 5AJS, 5F3K, 5F5R, 5HUZ, 5DJN, 5DJO, 5CHX, 5CJ0, 5CJ1, 5CJ4, 5C9N, 5CFF, 4WHV, 3WUT, 3WUU, 3WUV, 4ZQA, 4XA3, 4XA4, 4PXJ, 4YVC, 4YVE, SGML, 5AL7, 4WOT, 4CG4, 5AMO, 4WII, 4WIK, 4RSJ, 4CFG, 4R3Q, 4WID, 4CKG, 4CKH, 4NSW, 4W7P, 4QQ4, 4OJK, 4TL1, 40H9, 4LPZ, 4Q62, 4L2W, 4M3L, 4CKM, 4CKN, 4N6J, 4LTB, 4LRZ, 2MAJ, 2MAK, 4NAD, 4HW0, 4BT8, 4BT9, 4BTA, 4HHD, 4M8M, 4J3N, 4L6Q, 4C1A, 4C1B, 4GDO, 4BWK, 4BWP, 4BWX, 4HU5, 4HU6, 4L9U, 4G0U, 4G0V, 4G0W, 4L3I, 4G79, 4GEU, 4GEX, 4GFA, 4GFC, 4BL6, 4JMR, 4JNH, 2YMY, 4HAN, 3VMY, 3VMZ, 3VN0, 4ABX, 3W03, 2LW9, 4DZM, 4ETO, 3TNU, 3THF, 4E8U, 3VMX, 4E61, 3VEM, 3VBB, 4DJG, 3TV7, 3STQ, 3V8S, 3Q8T, 3U1C, 3QH9, 3AZD, 3ONX, 3OKQ, 3QX3, 3SJA, 3SJB, 3SJC, 2L2L, 3QFL, 3QKT, 2XV5, 2Y3W, 3QOX, 3AJW, 3NCZ, 3NI0, 2XU6, 3M91, 3NMD, 3LLL, 3LX7, 3ME9, 3MEU, 3MEV, 3ABH, 3ACO, 3IAO, 3HLS, 2WMM, 3A6M, 3A7O, 2WVR, 3ICX, 3ID5, 3ID6, 3HNW, 3I1G, 2K6S, 3GHG, 3G1E, 2W6A, 2V51, 3ERR, 3E1R, 2VY2, 2ZR2, 2ZR3, 3CL3, 3D9V, 2Z17, 2JEE, 3BBP, 3BAS, 3BAT, 2QM4, 2V71, 2NO2, 2PON, 2V0O, 2DQ0, 2DQ3, 2Q2F, 2NRN, 2E7S, 2H9V, 2FXM, 2HJD, 2GZD, 2GZH, 2FV4, 2F2U, 2EUL, 2ESM, 2ETK, 2ETR, 1ZXA, 1YIB, 1YIG, 1XSX, 1RFY, 1UO1, 1XJA, 1T3J, 1T6F, 1R7J, 1UII, 1PL5, 1S1C, 1P9I, 1R48, 1URU, 1OV9, 1UIX, 1NO4, 1NYH, 1MV4, 1LR1, 1L8D, 1LJ2, 1KQL, 1GXK, 1GXL, 1GK6, 1JR5, 1GMJ, 1JAD, 1JCH, 1J6G, 1JTH, 1JY2, 1JY3, 1IC2, 1HCl, 1HF9, 1HBW, 1FXK, 1D7M, 1QUU, 1CE9, 2A93, 1BM9, 1A93, 1TMZ, 2AAC, 1ZII, 1ZIK, 1ZIL, 2ARA, 2ARC, 1JUN, 1YSA, 2ZTA. However, this list of dimeric structures also contains antiparallel coiled coils since dimeric coiled coils with cyclic two-fold symmetry selects parallel and antiparallel coiled-coil. Visual inspection of the structure can easily tell apart the parallel from the antiparallel dimeric coiled coils.

Some of those entries for pentameric, tetrameric, trimeric and dimeric coiled coils also contain additional protein domains, but upon visual inspection those additional domains can easily be detected and removed.

As an alternative the website http://coiledcoils.chm.bris.ac.uk/ccplus/search/periodic-table/gives a periodic table of coiled-coil structures from which dimeric, trimeric, tetrameric and pentameric (such as 2GUV) coiled coils.

Amino acid modifications of these pentameric, tetrameric, trimeric and dimeric coiled coil domains are also envisaged. Such modifications may be e.g. the substitution of amino acids that are non-core residues (aa(a) and aa(d)) at the outside of the oligomer at positions aa(e), aa(g), aa(b), aa(c) or aa(f), preferably at positions aa(b), aa(c) or aa(f), most preferably in position aa(f). Possible modifications are substitutions to charged residues to make these oligomers more soluble. Also, shorter constructs of these domains are envisaged.

Other amino acid modifications may be e.g. the substitution of amino acids at core positions (aa(a) and aa(d)) for the purpose of stabilizing the oligomer, i.e. by replacing less favorable core residues by more favorable residues, i.e. as a general rule, residues at core positions with a lower coiled-coil propensity according to Table 1 can be replaced with residues with higher coiled-coil propensity if they do not change the oligomerization state of the coiled coil.

The term “amino acid modification” used herein includes an amino acid substitution, insertion, and/or deletion in a polypeptide sequence. By “amino acid substitution” or “substitution” herein is meant the replacement of an amino acid at a particular position in a parent polypeptide sequence with another amino acid. For example, the substitution R94K refers to a variant polypeptide, in which the arginine at position 94 is replaced with a lysine. For the purposes herein, multiple substitutions are typically separated by a slash. For example, R94K/L78V refers to a double variant comprising the substitutions R94K and L78V. By “amino acid insertion” or “insertion” as used herein is meant the addition of an amino acid at a particular position in a parent polypeptide sequence. For example, insert −94 designates an insertion at position 94. By “amino acid deletion” or “deletion” as used herein is meant the removal of an amino acid at a particular position in a parent polypeptide sequence. For example, R94− designates the deletion of arginine at position 94.

A peptide or protein containing an amino acid modification as described herein will preferably possess at least about 80%, most preferably at least about 90%, more preferably at least about 95%, in particular 99% amino acid sequence identity with a parent (un-modified) peptide or protein. Preferably the amino acid modification is a conservative modification.

As used herein, the term “conservative modification” or “conservative sequence modification” is intended to refer to amino acid modifications that do not significantly affect or alter the binding characteristics of the antibody containing the amino acid sequence. Such conservative modifications include amino acid substitutions, insertions and deletions. Modifications can be introduced into a protein of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

### Specific Coiled Coils

Most preferred are the coiled-coil sequences and monomeric building blocks described in the examples.

### Linkers

The linker connects the two coiled-coil oligomerization domains from the last core residue (either aa(a) or aa(d)) of the first oligomerization domain to the first core residue (either aa(a) or aa(d)) of the second coiled-coil oligomerization domain.

A peptide linker L1 and/or L2 is usually composed of a peptide chain with 3 to 50 amino acids, preferably with 3 to 10 amino acids, more preferably with 4 to 9 amino acids. In a preferred embodiment the peptide linker L1 and/or the peptide linker L2 independently from each other consists of at least two amino acids, of at least four amino acids, of at least five amino acids, of at least six amino acids, of at least seven amino acids, of at least eight amino acids, of at least nine amino acids, or of at least ten amino acids. In a more preferred embodiment the peptide linker L1 and/or the peptide linker L2 independently from each other consists of at least four amino acids, of at least seven amino acids, or of at least nine amino acids. In an even more preferred embodiment the peptide linker L1 and/or the peptide linker L2 independently from each other consists of at least four amino acids.

In a further preferred embodiment the peptide linker L1 and/or the peptide linker L2 independently from each other consists of two amino acids, four amino acids, five amino acids, six amino acids, seven amino acids, eight amino acids, nine amino acids, or ten amino acids. In a more preferred embodiment the peptide linker L1 and/or the peptide linker L2 independently from each other consists of four amino acids, seven amino acids, or nine amino acids. In an even more preferred embodiment the peptide linker L1 and/or the peptide linker L2 independently from each other consists of four amino acids.

In a particular embodiment the peptide linker L1 and/or the peptide linker L2 independently from each other comprises an amino acid sequence selected from the group consisting of the amino acid sequence as shown in SEQ ID NO:4, the amino acid sequence as shown in SEQ ID NO:12, the amino acid sequence as shown in SEQ ID NO: 14 and the amino acid sequence as shown in SEQ ID NO: 15, preferably the amino acid sequence as shown in SEQ ID NO: 4 and the amino acid sequence as shown in SEQ ID NO: 12, more preferably the amino acid sequence as shown in SEQ ID NO: 4.

The peptide linker L1 and/or L2 independently from each other usually contain between two and ten, preferably between three and seven positive charges at physiological conditions. Physiological conditions correspond to conditions in aqueous solution at a pH from 6.5 to 8.5, preferably at a pH of about 7.0 to 7.6. In a preferred embodiment the peptide linker L1 and/or the peptide linker L2 independently from each other contain at least two positive charges, at least three positive charges, at least four positive charges, at least five positive charges, at least six positive charges, at least seven positive charges, at least eight positive charges, at least nine positive charges, or at least ten positive charges. In a more preferred embodiment the peptide linker L1 and/or the peptide linker L2 independently from each other contain, at least three positive charges, at least five positive charges, or at least seven positive charges. In an even more preferred embodiment the peptide linker L1 and/or the peptide linker L2 independently from each other contain at least three positive charges.

In a further preferred embodiment the peptide linker L1 and/or the peptide linker L2 independently from each other contain two positive charges, three positive charges, four positive charges, five positive charges, six positive charges, seven positive charges, eight positive charges, nine positive charges, or ten positive charges. In a more preferred embodiment the peptide linker L1 and/or the peptide linker L2 independently from each other contain three positive charges, five positive charges, or seven positive charges. In an even more preferred embodiment the peptide linker L1 and/or the peptide linker L2 independently from each other contain three positive charges.

In a preferred embodiment the peptide linker L1 and/or the peptide linker L2 independently from each other contain at least one glycine residue such as RRGR (SEQ ID NO: 4) or KKGK (SEQ ID NO: 12).

In a preferred embodiment the peptide linker L1 and/or the peptide linker L2 independently from each other consists of at least four amino acids and has an overall positive charge of at least +3 at physiological conditions.

In a preferred embodiment the peptide linker L1 and the peptide linker L2 are identical.

### Nucleic Acid Derivatives

The term nucleic acid derivatives as used herein includes single-stranded DNA that contain a cytosine followed by a guanine wherein the cytosine nucleotide is unmethylated, single-stranded RNA from RNA viruses, double-stranded RNA from RNA viruses and polymeric complexes mimicking double-stranded RNA from RNA viruses.

A polymeric complex mimicking double-stranded RNA (dsRNA) is e.g. polyl:polyC (pIC), which is preferred. pIC is a large synthetic polymeric complex mimicking double-stranded RNA (dsRNA). Preparations of pIC vary in the distribution of the strand length, the solubility, and other biological properties including toxicity.

Single-stranded DNA that contains a cytosine followed by a guanine wherein the cytosine nucleotide is unmethylated is usually a CpG oligodeoxynucleotide (CpG ODN).

CpG oligodeoxynucleotide (CpG ODN) which are synthetic molecules differ from natural microbial DNA in that instead of the typical phosphodiester backbone they have a completely or partially phosphorothioated backbone and optionally a tail of poly G at the 5′ end, 3′ end. The poly G tail that forms intermolecular tetrads which result in high molecular weight aggregates thus enhancing cellular uptake while modification with phosphorothioate protects the ODN from being degraded by nucleases in vivo such as DNase.

Many different sequences have been shown to stimulate TLR9 that vary in the number and location of CpG dimers, as well as the exact base sequences flanking the CpG dimers. They can be classified in five unofficial classes or categories of CpG ODN. These classes are based on their sequence, secondary structures, and effect on human peripheral blood mononuclear cells (PBMCs) and are called Class A (Type D), Class B (Type K), Class C, Class P, and Class S.

Class A ODN are distinctly different from the Class B ODN in that it stimulates the production of large amounts of Type I interferons, the most important one being IFNα, and induced the maturation of plasmacytoid dendritic cells. Class A ODN are also strong activators of NK cells through indirect cytokine signaling. Class B ODN on the other hand are strong stimulators of human monocyte and B cell maturation. While they also stimulate the maturation of plasmacytoid dendritic cells they do this to a lesser extent than Class A ODN. They also stimulate very small amounts of IFNα.

### Class A

ODN 2216 is a class A CpG ODN and is a ligand of choice for human TLR9. It is a 20mer with the sequence 5′-ggGGGACGA:TCGTCgggggg-3′ (SEQ ID NO:43). Bases shown in capital letters are phosphodiester, and those in lower case are nuclease resistant phosphorothioates. The palindrome is underlined. ODN 2336 is another A-class CpG ODN with a preference for human TLR9. It is a 21mer with the sequence 5′-gggGACGAC:GTCGTGgggggg-3′ (SEQ ID NO:44).

### Class B

ODN 1826 is a class B CpG ODN specific for murine TLR9. It is a 20mer with the sequence 5′-tccatgacgttcctgacgtt-3′ (SEQ ID NO:13). All bases are nuclease resistant phosphorothioates. ODN 2006 is a class B CpG ODN and is a ligand of choice for human TLR9. It is a 24mer with the sequence 5′-tcgtcgttttgtcgttttgtcgtt-3′ (SEQ ID NO:42). ODN BW006 is a further type B CpG ODN and contains twice the optimal motif in human, GTCGTT. It is a 23mer with the sequence 5′-tcgacgttcgtcgttcgtcgttc-3′ (SEQ ID NO:45). Another type B CpG is ODN D-SL01. It is a TLR9 agonist in diverse vertebrate species, namely humans, mice, rats, rabbits, pigs and dogs and has the sequence 5′-tcgcgacgttcgcccgacgttcggta-3′ (SEQ ID NO:49) (26 mer).

### Class C

ODN 2395 is a CpG ODN class C specific for human and mouse TLR9. As a C-class CpG ODN it contains a complete phosphorothioate backbone and a CpG-containing palindromic motif. C-class CpG ODNs induce strong IFN-α production from pDC and B cell stimulation. It is a 22mer with the sequence 5′-tcgtcgttttcggcgc:gcgccg-3′ (SEQ ID NO:46). All bases are phosphorothioate and palindrome is underlined. ODN M362 is another CpG ODN class C specific for human and mouse TLR9. It is a 25mer with the sequence 5′-tcgtcgttc:gaacgacgttgat-3′ (SEQ ID NO:47). Another type C CpG ODN is ODN D-SL03. It is a TLR9 agonist in diverse vertebrate species, namely humans, mice, rats, rabbits, pigs and dogs. ODN D-SL03 is composed of double stem loops, a phosphorothioate backbone and two palindromes with AACGTT motif and TTCGAA motif in each loop. ODN D-SL03 is a robust inducer of IFN-α apparently due to the presence of the palindrome sequence. D-SL03 has been shown to potently activate human B cells, NK cells and mononuclear cells as well as PBMC/splenocytes obtained from diverse vertebrate species, namely mice, rats, rabbits, dogs and pigs. ODN D-SL03 demonstrates anti-tumor activity in mice with established breast cancer. It is a 29mer with the sequence 5′-tcgcgaacgttcgccgcgttcgaacgcgg-3′ (SEQ ID NO:48).

In a preferred embodiment the nucleic acid derivative is a CpG oligodeoxynucleotide (CpG ODN). In a preferred embodiment the nucleic acid derivative is a CpG oligodeoxynucleotide (CpG ODN) wherein at least one nucleotide, preferably at least one cytosine nucleotide in a CpG motif is unmethylated. In a preferred embodiment the nucleic acid derivative is a CpG oligodeoxynucleotide (CpG ODN) wherein between one and ten, preferably between two and eight, more preferably between two and five cytosine nucleotides in CpG motifs are unmethylated.

In an even more preferred embodiment the nucleic acid derivative is a CpG oligodeoxynucleotide (CpG ODN) selected from the group consisting of Class A CpG ODN, Class B CpG ODN and Class C CpG ODN. In a particular preferred embodiment the nucleic acid derivative is a CpG oligodeoxynucleotide (CpG ODN) selected from the group consisting of the nucleotide acid sequence as shown in SEQ ID NO:13, SEQ ID NO:39, SEQ ID NO:42, SEQ ID NO:43, SEQ ID NO:44, SEQ ID NO:45, SEQ ID NO:46, SEQ ID NO:47, SEQ ID NO:48 and SEQ ID NO:49, in particular the nucleic acid derivative is a CpG oligodeoxynucleotide (CpG ODN) is selected from the group consisting of the nucleotide acid sequence as shown in SEQ ID NO:13 and the nucleotide acid sequence as shown in SEQ ID NO:39.

In the composition according to the invention the nucleic acid derivative is not covalently bound to the SAPN i.e. the nucleic acid derivative is bound to the SAPN by ionic interactions.

Usually the nucleic acid derivative is bound to the peptide linker L1 and/or L2 by ionic interactions.

### Self-Assembling Protein Nanoparticles: LCM Units

SAPNs are formed from monomeric building blocks of formula (I) optionally co-assembled with monomeric building blocks of formula (II). If such building blocks assemble, they will form so-called “LCM units”. The number of monomeric building blocks, which will assemble into such an LCM unit will be defined by the least common multiple (LCM). Hence, if for example the oligomerization domains of the monomeric building block form a pentamer (ND1)₅ (m=5) and a trimeric (ND2)₃ (n=5), 15 monomers will form an LCM unit. If the linker segments L1 and L2 have the appropriate length, this LCM unit may assemble in the form of a spherical protein nanoparticle. SAPNs may be formed by the assembly of only one or more than one LCM units (Table 2). Such SAPNs represent topologically closed structures.

### Regular Polyhedra

There exist five regular polyhedra, the tetrahedron, the cube, the octahedron, the dodecahedron and the icosahedron. They have different internal rotational symmetry elements. The tetrahedron has a 2-fold and two 3-fold axes, the cube and the octahedron have a 2-fold, a 3-fold and a 4-fold rotational symmetry axis, and the dodecahedron and the icosahedron have a 2-fold, a 3-fold and a 5-fold rotational symmetry axis. In the cube the spatial orientation of these axes is exactly the same as in the octahedron, and also in the dodecahedron and the icosahedron the spatial orientation of these axes relative to each other is exactly the same. Hence, for the purpose of SAPNs of the invention the dodecahedron and the icosahedron can be considered to be identical. The dodecahedron/icosahedron is built up from 60 identical three-dimensional building blocks (Table 1). These building blocks are the asymmetric units (AUs) of the polyhedron. They are pyramids and the pyramid edges correspond to one of the rotational symmetry axes, hence these AUs will carry at their edges 2-fold, 3-fold, and 5-fold symmetry elements. If these symmetry elements are generated from protein oligomerization domains such AUs are constructed from monomeric building blocks as described above. It is sufficient to align the two oligomerization domains ND1 and ND2 or ND3 and ND4 along two of the symmetry axes of the AU. If these two oligomerization domains form stable oligomers, the symmetry interface along the third symmetry axis will be generated automatically, and it may be stabilized by optimizing interactions along this interface, e.g. hydrophobic, hydrophilic or ionic interactions, or covalent bonds such as disulfide bridges.

In a preferred embodiment at least one of the oligomerization domains ND1, ND2, ND3 and ND4, preferably either ND1 and/or ND3 or ND2 and/or ND4 of formula (I) or (II) comprises a dimeric, a trimeric, a tetrameric and/or a pentameric domain, more preferably a dimeric, a tetrameric and/or a pentameric domain, even more preferably a dimeric and/or a pentameric domain.

In a more preferred embodiment one of the oligomerization domains ND1, ND2, ND3 and/or ND4 of formula (I) or (II), more preferably either ND1 and/or ND3 or ND2 and/or ND4comprises a pentameric coiled coil selected from the group consisting of 4PN8, 4PND, 4WBA, 3V2N, 3V2P, 3V2Q, 3V2R, 4EEB, 4EED, 3MIW, 1MZ9, 1FBM, 1VDF, 2GUV, 2HYN, 1ZLL, and 1T8Z or a pentameric coiled coil selected from the group consisting of 4PN8, 4PND, 4WBA, 3V2N, 3V2P, 3V2Q, 3V2R, 4EEB, 4EED, 3MIW, 1MZ9, 1FBM, 1VDF, 2GUV, 2HYN, 1ZLL, and 1T8Z, which contains an amino acid modification and/or is shortened at either or both ends wherein each coiled coil is indicated according to the pdb entry numbering of the RCSB Protein Data Bank (RCSB PDB). Even more preferably ND1 is a pentameric coiled coil selected from the group consisting of the tryptophan-zipper pentamerization domain (pdb-entry: 1T8Z) or a tryptophan-zipper pentamerization domain (pdb-entry: 1T8Z) contains an amino acid modification and/or is shortened at either or both ends, in particular a pentameric coiled coil comprising SEQ ID NO: 3 or SEQ ID NO: 25) or a pentameric coiled coil comprising SEQ ID NO: 3 or SEQ ID NO: 25 with amino acid modifications and/or shortened at either or both ends,

In another more preferred embodiment at least one of the oligomerization domains ND1, ND2, ND3 and ND4 of formula (I) or (II) more preferably either ND1 and/or ND3 or ND2 and/or ND4 comprises a tetrameric coiled coil selected from the group consisting of tetrameric coiled coil 5D60, 5D5Y, 5AL6, 4WB4, 4BHV, 4C5Q, 4GJW, 4H7R, 4H8F, 4BXT, 4LTO, 4LTP, 4LTQ, 4LTR, 3ZDO, 3RQA, 3R4A, 3R4H, 3TSI, 3K4T, 3F6N, 2O6N, 2OVC, 2O1J, 2OK, 2AG3, 2CCE, 1YBK, 1U9F, 1U9G, 1U9H, 1USD, 1USE, 1UNT, 1UNU, 1UNV, 1UNW, 1UNX, 1UNY, 1UNZ, 1UO0, 1UO1, 1UO2, 1UO3, 1UO4, 1UO5, 1W5I, 1W5L, 1FE6, 1G1I, 1G1J, 1EZJ, 1RH4, 1GCL or a tetrameric coiled coil selected from the group consisting of 5D60, 5D5Y, 5AL6, 4WB4, 4BHV, 4C5Q, 4GJW, 4H7R, 4H8F, 4BXT, 4LTO, 4LTP, 4LTQ, 4LTR, 3ZDO, 3RQA, 3R4A, 3R4H, 3TS1, 3K4T, 3F6N, 2O6N, 2OVC, 2O1J, 2OK, 2AG3, 2CCE, 1YBK, 1U9F, 1U9G, 1U9H, 1USD, 1USE, 1UNT, 1UNU, 1UNV, 1UNW, 1UNX, 1UNY, 1UNZ, 1UO0, 1UO1, 1UO2, 1UO3, 1UO4, 1UO5, 1W5I, 1W5L, 1FE6, 1G1I, 1G1J, 1EZJ, 1RH4, 1GCL, which contains an amino acid modification and/or is shortened at either or both ends wherein each coiled coil is indicated according to the pdb entry numbering of the RCSB Protein Data Bank (RCSB PDB).

In a most preferred embodiment the tetrameric coiled coil is from tetrabrachion (pdb-entry code 1FE6) or the tetrameric coiled coil is from tetrabrachion (pdb-entry code 1FE6) which contains an amino acid modification and/or is shortened at either or both ends, wherein each SHB is indicated according to the pdp entry numbering of the RCSB Protein Data Bank (RCSB PDB).

In another more preferred embodiment one of the oligomerization domains ND1, ND2, ND3 and ND4 of formula (I) or (II) more preferably either ND1 and/or ND3 or ND2 and/or ND4 comprises a trimeric coiled coil selected from the group consisting of trimeric coiled coil 5TOH, 5TO1, 5K92, 5KB0, 5KB1, 5KB2, 5KKV, 5EFM, 2N64, 5ABS, 5IEA, 5APP, 5APQ, 5APS, 5APY, 5APZ, 5D5Z, 4YPC, 4YV3, 4CGB, 4CGC, 4CJD, 4R0R, 4UW0, 4P67, 4OXM, 3W8V, 3W92, 3W93, 4I2L, 4K8U, 4JBZ, 3VTQ, 4L1R, 4JDO, 4J4A, 4E52, 3VYI, 3ZMF, 3VU5, 3VU6, 2YNY, 2YNZ, 2YO0, 2YO1, 2YO2, 4G1A, 4GIF, 3TQ2, 4DZK, 4DZL, 4DZN, 3TE3, 3R48, 3SWF, 3SWY, 3PR7, 2YKO, 2YKP, 2YKQ, 3NTN, 3PP5, 3MKO, 3MGN, 3NWA, 3NWD, 3NWF, 3L35, 3L36, 3L37, 3M9B, 3M9D, 2X6P, 3LJM, 3AHA, 3H7X, 3H7Z, 3LT6, 3LT7, 3GJP, 2KP8, 3KPE, 2WPR, 2WPS, 2WPY, 2WPZ, 2WQ0, 2WQ1, 2WQ2, 2WQ3, 3HFC, 3HFE, 3HRN, 3HRO, 3H5F, 3H5G, 2WG5, 2WG6, 2W6B, 2JJL, 2VRS, 3EFG, 3DUZ, 2OT5, 2Z2T, 2QIH, 36K6, 207H, 2R32, 2JGO, 2Q7C, 2Q3I, 2Q5U, 2IBL, 1ZV8, 1ZVB, 2FXP, 1WT6, 2AKF, 1TGG, 1SLQ, 1S9Z, 1PW9, 1PWB, 1M7L, 1GZL, 1KYC, 1KFM, 1KFN, 1J0, 1IJ1, 1IJ2, 1IJ3, 1HQJ, 1QU1, 1B08, 1CZQ, 1CUN, 1SVF, 1CE0, 1PIQ, 1AQ5, 1AVY, 1HTN, 1AA0, 1ZIJ, 1ZIM, COI, 1SWI, 1GCM, 1HUP or a trimeric coiled coil selected from the group consisting of 5TOH, 5TOI, 5K92, 5KB0, 5KB1, 5KB2, 5KKV, 5EFM, 2N64, 5ABS, 5IEA, 5APP, 5APQ, 5APS, 5APY, 5APZ, 5D5Z, 4YPC, 4YV3, 4CGB, 4CGC, 4CJD, 4R0R, 4UW0, 4P67, 4OXM, 3W8V, 3W92, 3W93, 4I2L, 4K8U, 4JBZ, 3VTQ, 4L1R, 4JDO, 4J4A, 4E52, 3VYI, 3ZMF, 3VU5, 3VU6, 2YNY, 2YNZ, 2YO0, 2YO1, 2YO2, 4G1A, 4GIF, 3TQ2, 4DZK, 4DZL, 4DZN, 3TE3, 3R48, 3SWF, 3SWY, 3PR7, 2YKO, 2YKP, 2YKQ, 3NTN, 3PP5, 3MKO, 3MGN, 3NWA, 3NWD, 3NWF, 3L35, 3L36, 3L37, 3M9B, 3M9D, 2X6P, 3LJM, 3AHA, 3H7X, 3H7Z, 3LT6, 3LT7, 3GJP, 2KP8, 3KPE, 2WPR, 2WPS, 2WPY, 2WPZ, 2WQ0, 2WQ1, 2WQ2, 2WQ3, 3HFC, 3HFE, 3HRN, 3HRO, 3H5F, 3H5G, 2WG5, 2WG6, 2W6B, 2JJL, 2VRS, 3EFG, 3DUZ, 2OT5, 2Z2T, 2QIH, 36K6, 207H, 2R32, 2JGO, 2Q7C, 2Q3I, 2Q5U, 2IBL, 1ZV8, 1ZVB, 2FXP, 1WT6, 2AKF, 1TGG, 1SLQ, 1S9Z, 1PW9, 1PWB, 1M7L, 1GZL, 1KYC, 1KFM, 1KFN, 1J0, 1IJ1, 1IJ2, 1IJ3, 1HQJ, 1QU1, 1B08, 1CZQ, 1CUN, 1SVF, 1CE0, 1PIQ, 1AQ5, 1AVY, 1HTN, 1AA0, 1ZIJ, 1ZIM, 1COI, 1SWI, 1GCM, 1HUP, which contains an amino acid modification and/or is shortened at either or both ends wherein each coiled coil is indicated according to the pdb entry numbering of the RCSB Protein Data Bank (RCSB PDB)

In another more preferred embodiment one of the oligomerization domains ND1, ND2, ND3 and ND4 of formula (1) or (11) more preferably either ND1 and/or ND3 or ND2 and/or ND4 comprises a dimeric coiled coil selected from the group consisting of dimeric coiled coil 5M97, 5M9E, 5FIY, 5F4Y, 5D3A, 5HMO, 5EYA, 5IX1, 5IX2, 5JHF, 5JVM, 5JVP, 5JVR, 5JVS, 5JVU, 5JX1, 5FCN, 5HHE, 2N9B, 4ZRY, 4Z6Y, 4YTO, 4ZI3, 5AJS, 5F3K, 5F5R, 5HUZ, 5DJN, 5DJO, 5CHX, 5CJ0, 5CJ1, 5CJ4, 5C9N, 5CFF, 4WHV, 3WUT, 3WUU, 3WUV, 4ZQA, 4XA3, 4XA4, 4PXJ, 4YVC, 4YVE, SGML, 5AL7, 4WOT, 4CG4, 5AMO, 4WII, 4WIK, 4RSJ, 4CFG, 4R3Q, 4WID, 4CKG, 4CKH, 4NSW, 4W7P, 4QQ4, 4OJK, 4TL1, 4OH9, 4LPZ, 4Q62, 4L2W, 4M3L, 4CKM, 4CKN, 4N6J, 4LTB, 4LRZ, 2MAJ, 2MAK, 4NAD, 4HW0, 4BT8, 4BT9, 4BTA, 4HHD, 4M8M, 4J3N, 4L6Q, 4C1A, 4C1B, 4GDO, 4BWK, 4BWP, 4BWX, 4HU5, 4HU6, 4L9U, 4G0U, 4G0V, 4G0W, 4L3I, 4G79, 4GEU, 4GEX, 4GFA, 4GFC, 4BL6, 4JMR, 4JNH, 2YMY, 4HAN, 3VMY, 3VMZ, 3VNO, 4ABX, 3W03, 2LW9, 4DZM, 4ETO, 3TNU, 3THF, 4E8U, 3VMX, 4E61, 3VEM, 3VBB, 4DJG, 3TV7, 3STQ, 3V8S, 3Q8T, 3U1C, 3QH9, 3AZD, 3ONX, 3OKQ, 3QX3, 3SJA, 3SJB, 3SJC, 2L2L, 3QFL, 3QKT, 2XV5, 2Y3W, 3QOX, 3AJW, 3NCZ, 3NI0, 2XU6, 3M91, 3NMD, 3LLL, 3LX7, 3ME9, 3MEU, 3MEV, 3ABH, 3ACO, 3IAO, 3HLS, 2WMM, 3A6M, 3A7O, 2WVR, 3ICX, 3ID5, 3ID6, 3HNW, 3I1G, 2K6S, 3GHG, 3G1E, 2W6A, 2V51, 3ERR, 3E1R, 2VY2, 2ZR2, 2ZR3, 3CL3, 3D9V, 2Z17, 2JEE, 366P, 3BAS, 3BAT, 2QM4, 2V71, 2NO2, 2PON, 2V0O, 2DQ0, 2DQ3, 2Q2F, 2NRN, 2E7S, 2H9V, 2FXM, 2HJD, 2GZD, 2GZH, 2FV4, 2F2U, 2EUL, 2ESM, 2ETK, 2ETR, 1ZXA, 1YIB, 1YIG, 1XSX, 1RFY, 1UO1, 1XJA, 1T3J, 1T6F, 1R7J, 1UII, 1PL5, 1S1C, 1P9I, 1R48, 1URU, 1OV9, 1UIX, 1NO4, 1NYH, 1MV4, 1LR1, 1L8D, 1LJ2, 1KQL, 1GXK, 1GXL, 1GK6, 1JR5, 1GMJ, 1JAD, 1JCH, 1J6G, 1JTH, 1JY2, 1JY3, 1IC2, 1HCl, 1HF9, 1HBW, 1FXK, 1D7M, 1QUU, 1CE9, 2A93, 1BM9, 1A93, 1TMZ, 2AAC, 1Z1I, 1ZIK, 1ZIL, 2ARA, 2ARC, 1JUN, 1YSA, 2ZTA or a dimeric coiled coil selected from the group consisting of 5M97, 5M9E, 5FIY, 5F4Y, 5D3A, 5HMO, 5EYA, 5IX1, 5IX2, 5JHF, 5JVM, 5JVP, 5JVR, 5JVS, 5JVU, 5JX1, 5FCN, 5HHE, 2N9B, 4ZRY, 4Z6Y, 4YTO, 4ZI3, 5AJS, 5F3K, 5F5R, 5HUZ, 5DJN, 5DJO, 5CHX, 5CJ0, 5CJ1, 5CJ4, 5C9N, 5CFF, 4WHV, 3WUT, 3WUU, 3WUV, 4ZQA, 4XA3, 4XA4, 4PXJ, 4YVC, 4YVE, 5BML, 5AL7, 4WOT, 4CG4, SAMO, 4W11, 4WIK, 4RSJ, 4CFG, 4R3Q, 4WID, 4CKG, 4CKH, 4NSW, 4W7P, 4QQ4, 4OJK, 4TL1, 40H9, 4LPZ, 4Q62, 4L2W, 4M3L, 4CKM, 4CKN, 4N6J, 4LTB, 4LRZ, 2MAJ, 2MAK, 4NAD, 4HWO, 4BT8, 4BT9, 4BTA, 4HHD, 4M8M, 4J3N, 4L6Q, 4C1A, 4C1B, 4GDO, 4BWK, 4BWP, 4BWX, 4HU5, 4HU6, 4L9U, 4G0U, 4G0V, 4G0W, 4L3I, 4G79, 4GEU, 4GEX, 4GFA, 4GFC, 4BL6, 4JMR, 4JNH, 2YMY, 4HAN, 3VMY, 3VMZ, 3VNO, 4ABX, 3W03, 2LW9, 4DZM, 4ETO, 3TNU, 3THF, 4E8U, 3VMX, 4E61, 3VEM, 3VBB, 4DJG, 3TV7, 3STQ, 3V8S, 3Q8T, 3U1C, 3QH9, 3AZD, 3ONX, 3OKQ, 3QX3, 3SJA, 3SJB, 3SJC, 2L2L, 3QFL, 3QKT, 2XV5, 2Y3W, 3QOX, 3AJW, 3NCZ, 3NI0, 2XU6, 3M91, 3NMD, 3LLL, 3LX7, 3ME9, 3MEU, 3MEV, 3ABH, 3ACO, 3IAO, 3HLS, 2WMM, 3A6M, 3A7O, 2WVR, 3ICX, 3ID5, 3ID6, 3HNW, 3I1G, 2K6S, 3GHG, 3G1E, 2W6A, 2V51, 3ERR, 3E1R, 2VY2, 2ZR2, 2ZR3, 3CL3, 3D9V, 2Z17, 2JEE, 3BBP, 3BAS, 3BAT, 2QM4, 2V71, 2NO2, 2PON, 2V0O, 2DQ0, 2DQ3, 2Q2F, 2NRN, 2E7S, 2H9V, 2FXM, 2HJD, 2GZD, 2GZH, 2FV4, 2F2U, 2EUL, 2ESM, 2ETK, 2ETR, 1ZXA, 1YIB, 1YIG, 1XSX, 1RFY, 1U01, 1XJA, 1T3J, 1T6F, 1R7J, 1UII, 1PL5, 1S1C, 1P9I, 1R48, 1URU, 10V9, 1UIX, 1NO4, 1NYH, 1MV4, 1LR1, 1L8D, 1LJ2, 1KQL, 1GXK, 1GXL, 1GK6, 1JR5, 1GMJ, 1JAD, 1JCH, 1JBG, 1JTH, 1JY2, 1JY3, 1IC2, 1HCl, 1HF9, 1HBW, 1FXK, 1D7M, 1QUU, 1CE9, 2A93, 1BM9, 1A93, 1TMZ, 2AAC, 1Z1I, 1ZIK, 1ZIL, 2ARA, 2ARC, 1JUN, 1YSA, 2ZTA, which contains an amino acid modification and/or is shortened at either or both ends wherein each coiled coil is indicated according to the pdb entry numbering of the RCSB Protein Data Bank (RCSB PDB).

In a preferred embodiment X1 is selected from the group consisting of an amino acid sequence comprising a Histag, an amino acid sequence comprising the Histag as shown in SEQ ID NO: 29, an amino acid sequence comprising a Histag and the cell-traversal protein of Plasmodium ookinetes and sporozoites (CeITOS), an amino acid sequence comprising a Histag and the cell-traversal protein of Plasmodium ookinetes and sporozoites (CeITOS) as shown in SEQ ID NO: 30, an amino acid sequence as shown in SEQ ID NO: 2, an amino acid sequence as shown in SEQ ID NO: 29, an amino acid sequence as shown in SEQ ID NO: 24, and an amino acid sequence as shown in SEQ ID NO: 2, SEQ ID NO: 30, SEQ ID NO: 29 or SEQ ID NO: 24, wherein the amino acid sequence contains an amino acid modification and/or is shortened at either or both ends. More preferably X1 is selected from the group consisting of an amino acid sequence as shown in SEQ ID NO: 2, an amino acid sequence as shown in SEQ ID NO: 29, an amino acid sequence as shown in SEQ ID NO: 24, and an amino acid sequence as shown in SEQ ID NO: 2, SEQ ID NO: 29 or SEQ ID NO: 24, wherein the amino acid sequence contains an amino acid modification and/or is shortened at either or both ends.

In a preferred embodiment X2 is selected from the group consisting of an amino acid sequence comprising a Histag, an amino acid sequence comprising the Histag as shown in SEQ ID NO: 29, an amino acid sequence comprising a Histag and the cell-traversal protein of Plasmodium ookinetes and sporozoites (CeITOS), an amino acid sequence comprising a Histag and the cell-traversal protein of Plasmodium ookinetes and sporozoites (CeITOS) as shown in SEQ ID NO: 30, an amino acid sequence as shown in SEQ ID NO: 2, an amino acid sequence as shown in SEQ ID NO: 29, an amino acid sequence as shown in SEQ ID NO: 24, and an amino acid sequence as shown in SEQ ID NO: 2, SEQ ID NO: 30, SEQ ID NO: 29 or SEQ ID NO: 24, wherein the amino acid sequence contains an amino acid modification and/or is shortened at either or both ends. More preferably X1 is selected from the group consisting of an amino acid sequence as shown in SEQ ID NO: 2, an amino acid sequence as shown in SEQ ID NO: 29, an amino acid sequence as shown in SEQ ID NO: 24, and an amino acid sequence as shown in SEQ ID NO: 2, SEQ ID NO: 29 or SEQ ID NO: 24, wherein the amino acid sequence contains an amino acid modification and/or is shortened at either or both ends.

In a preferred embodiment Y1 is selected from the group consisting of an amino acid sequence comprising the cell-traversal protein of Plasmodium ookinetes and sporozoites (CeITOS), an amino acid sequence as shown in SEQ ID NO: 27, and an amino acid sequence as shown in SEQ ID NO: 27, wherein the amino acid sequence contains an amino acid modification and/or is shortened at either or both ends.

In a preferred embodiment Y2 is an amino acid sequence comprising the D0 and D1 domains of flagellin, an amino acid sequence as shown in SEQ ID NO: 28 or SEQ ID NO: 6 or an amino acid sequence as shown SEQ ID NO: 28 or SEQ ID NO: 6, wherein the amino acid sequence contains an amino acid modification and/or is shortened at either or both ends.

In a preferred embodiment the peptide linker L1 consists of at least three amino acids and at least one, preferably at least two, more preferably at least three, even more preferably all of X1, ND1, ND2 and Y1 of the building block of formula (I) are selected from the group consisting of X1 as shown in SEQ ID NO:2 or in SEQ ID NO: 24; ND1 as shown in SEQ ID NO: 3 or in SEQ ID NO: 25; ND2 as shown in SEQ ID NO: 5 or in SEQ ID NO: 26; and Y1 as shown in SEQ ID NO: 6 or in SEQ ID NO:27 or the peptide linker L1 consists of at least three amino acids and at least one, preferably at least two, more preferably at least three, even more preferably all of X1, ND1, ND2 and Y1 of the building block of formula (I) are selected from the group consisting of X1 as shown in SEQ ID NO:2 or in SEQ ID NO: 24; ND1 as shown in SEQ ID NO: 3 or in SEQ ID NO: 25; ND2 as shown in SEQ ID NO: 5 or in SEQ ID NO: 26; and Y1 as shown in SEQ ID NO: 6 or in SEQ ID NO:27, wherein at least one of SEQ ID NO:2, SEQ ID NO: 24, SEQ ID NO: 3, SEQ ID NO: 25, SEQ ID NO: 5, SEQ ID NO: 26, SEQ ID NO: 6 or SEQ ID NO:27 contains an amino acid modification and/or is shortened at either or both ends.

In a preferred embodiment the peptide linker L2 consists of at least three amino acids and at least one, preferably at least two, more preferably at least three, even more preferably all of X2, ND3, ND4 and Y2 of the building block of formula (I) are selected from the group consisting of X2 as shown in SEQ ID NO:24; ND3 as shown in SEQ ID NO: 25; ND4 as shown in SEQ ID NO: 26; and Y2 as shown in SEQ ID NO:28 or wherein the peptide linker L2 consists of at least three amino acids and at least one, preferably at least two, more preferably at least three, even more preferably all of X2, ND3, ND4 and Y2 of the building block of formula (I) are selected from the group consisting of X2 as shown in SEQ ID NO:24; ND3 as shown in SEQ ID NO: 25; ND4 as shown in SEQ ID NO: 26; and Y2 as shown in SEQ ID NO:28, wherein at least one of SEQ ID NO:24; SEQ ID NO: 25; SEQ ID NO: 26 or SEQ ID NO:28 contains an amino acid modification and/or is shortened at either or both ends.

In a preferred embodiment the building block of formula (I) comprises a continuous chain of amino acids selected from the group consisting of the amino acid sequence as shown in SEQ ID NO: 1, the amino acid sequence as shown in SEQ ID NO: 16, the amino acid sequence as shown in SEQ ID NO: 17, the amino acid sequence as shown in SEQ ID NO: 18, the amino acid sequence as shown in SEQ ID NO: 19, the amino acid sequence as shown in SEQ ID NO: 22 and the amino acid sequence as shown in SEQ ID NO: 34 or the building block of formula (I) comprises a continuous chain of amino acids selected from the group consisting of the amino acid sequence as shown in SEQ ID NO: 1, the amino acid sequence as shown in SEQ ID NO: 16, the amino acid sequence as shown in SEQ ID NO: 17, the amino acid sequence as shown in SEQ ID NO: 18, the amino acid sequence as shown in SEQ ID NO: 19, the amino acid sequence as shown in SEQ ID NO: 22 and the amino acid sequence as shown in SEQ ID NO: 34, at least one SEQ ID NO:16; SEQ ID NO: 17; SEQ ID NO: 18, SEQ ID NO: 19, SEQ ID NO: 22 or SEQ ID NO: 34 contains an amino acid modification and/or is shortened at either or both ends.

In a preferred embodiment the building block of formula (II) comprises a continuous chain of amino acids as shown in SEQ ID NO: 23 or the building block of formula (II) comprises a continuous chain of amino acids as shown in SEQ ID NO: 23, wherein the amino acid as shown in SEQ ID NO: 23 contains an amino acid modification and/or is shortened at either or both ends.

In a preferred embodiment the molar ratio of the protein chains of SAPN consisting of a multitude of building blocks of formula (I), a multitude of building blocks of formula (II) or a multitude of co-assembled building blocks of formula (I) and formula (II), more preferably of the protein chains of SAPN consisting of a multitude of building blocks of formula (I) to the nucleic acid derivative is about 1 to about 0.4 to 0.8, preferably about 1 to about 0.6.

In a preferred embodiment the composition comprises a SAPN consisting of a multitude of building blocks of formula (I) co-assembled with a multitude of building blocks of formula (II).

In a preferred embodiment the co-assembled SAPN comprising a multitude of building blocks of formula (I) and a multitude of building blocks of formula (II), more preferably the co-assembled SAPN comprising a multitude of building blocks of formula (I) and a multitude of building blocks of formula (II) comprising a flagellin as described herein, has a co-assembly ratio of about 48 to about 59 of the continuous chain comprising a building block of formula (I) to about 12 to about 1 of the continuous chain comprising a building block of formula (II), more preferably about 55 to about 58 of the continuous chain comprising a building block of formula (I) to about 5 to about 2 of the continuous chain comprising a building block of formula (II), e.g. about 55 of the continuous chain comprising a building block of formula (I) to about 5 of the continuous chain comprising a building block of formula (II), about 56 of the continuous chain comprising a building block of formula (I) to about 4 of the continuous chain comprising a building block of formula (II), about 57 of the continuous chain comprising a building block of formula (I) to about 3 of the continuous chain comprising a building block of formula (II), or about 58 of the continuous chain comprising a building block of formula (I) to about 2 of the continuous chain comprising a building block of formula (II), even more preferably about 58 of the continuous chain comprising a building block of formula (I) to about 2 of the continuous chain comprising a building block of formula (II).

### Assembly to Self-Assembling Protein Nanoparticles (SAPNs) with Regular Polyhedral Symmetry

To generate self-assembling protein nanoparticles (SAPNs) with a regular geometry (dodecahedron, icosahedron, octahedron, cube and tetrahedron), more than one LCM unit is needed. E.g. to form an icosahedron from a monomer containing trimeric and pentameric oligomerization domains, 4 LCM units, each composed of 15 monomeric building blocks are needed, i.e. the protein nanoparticle with regular geometry will be composed of 60 monomeric building blocks. The combinations of the oligomerization states of the two oligomerization domains needed and the number of LCM units to form the corresponding polyhedra are listed in Table 2.

TABLE 2
Possible combinations of oligomerization states
in the formation of regular polyhedra

| | | | | | No. of | No. of |
|---|---|---|---|---|---|---|
| | | | | | Even | Building |
| ID No. | m | n | Polyhedron Type | LCM | Units | Blocks |
| 1 | 5 | 2 | dodecahedron/icosahedrons | 10 | 6 | 60 |
| 2 | 5 | 3 | dodecahedron/icosahedrons | 15 | 4 | 60 |
| 3 | 4 | 3 | cube/octahedron | 12 | 2 | 24 |
| 4 | 3 | 4 | cube/octahedron | 12 | 2 | 24 |
| 5 | 3 | 5 | dodecahedron/icosahedrons | 15 | 4 | 60 |
| 6 | 2 | 5 | dodecahedron/icosahedrons | 10 | 6 | 60 |
| 7 | 5 | 4 | Irregular | 20 | 1 | 20 |
| 8 | 4 | 5 | Irregular | 20 | 1 | 20 |

Whether the LCM units will further assemble to form regular polyhedra composed of more than one LCM unit depends on the geometrical alignment of the two oligomerizations domains ND1 and ND2 and of the two oligomerizations domains ND3 and ND4, respectively, with respect to each other, especially on the angle between the rotational symmetry axes of the two oligomerization domains. This is mainly governed by i) the interactions between neighboring domains in a nanoparticle, ii) the length of the linker segment L1 and L2, iii) the shape of the individual oligomerization domains. This angle is larger in the LCM units compared to the arrangement in a regular polyhedron. Also this angle is not identical in monomeric building blocks as opposed to the regular polyhedron.

If the angle between the two oligomerization domains is sufficiently small (even smaller than in a regular polyhedron with icosahedral symmetry), then a large number (several hundred) protein chains can assemble into a protein nanoparticle. A biophysical and mathematical analysis of SAPNs with trimer-pentamer architecture has recently been published (Indelicato, G., et al. Biophys J 2016, 110(3): 646-660).

Preferably, antigens to be displayed in a loop-conformation on the SAPNs are selected from the group consisting of: (a) proteins or peptides suited to induce an immune response against cancer cells; (b) proteins or peptides suited to induce an immune response against infectious diseases; (c) proteins or peptides suited to induce an immune response against allergens; (d) proteins or peptides suited to induce an immune response for the treatment of a human disease.

SAPNs comprising such proteins or peptides may be suited to induce an immune response in humans, or also in farm animals and pets.

In a further aspect, the invention relates to monomeric building blocks of formula (I) or (II) as defined above.

In another aspect, the invention relates to a composition comprising a protein nanoparticle as herein described suitable as a vaccine e.g. a composition comprising a protein nanoparticle as herein described for use as a vaccine. Preferred vaccine compositions comprise the protein nanoparticle in an aqueous buffer solution, and may further comprise, for example, sugar derived excipients (such as glycerol, trehalose, sucrose, etc.) or amino acid derived excipients (such as arginine, proline, glutamate, etc.) or anionic, cationic, non-ionic or twitter-ionic detergents (such as cholate, deoxycholate, tween, etc.) or any kind of salt (such as NaCl, MgCl₂, etc.) to adjust the ionic strength of the solution.

In another aspect, the invention relates to a method of vaccinating a human or non-human animal, which comprises administering an effective amount of a composition as described hereinbefore to a subject in need of such vaccination. The subject in need of such vaccination is usually a human or non-human animal.

Also provided is a composition as described hereinbefore for use in a method of vaccinating a human or non-human animal, the method comprising administering an effective amount of said composition to a human or non-human animal in need of such vaccination.

Also provided is the use of a composition as described hereinbefore for the manufacture of a medicament for vaccinating a human or non-human animal.

Also provided is the use of a composition as described hereinbefore for vaccinating a human or non-human animal.

The terms “individual,” “subject” or “patient” are used herein interchangeably. In certain embodiments, the subject is a mammal. Mammals include, but are not limited to primates (including human and non-human primates). In a preferred embodiment, the subject is a human. In a further aspect the invention relates to a method of producing a SAPN as described herein, comprising i) adding a SAPN to a buffer comprising a nucleic acid derivative and ii) refolding the SAPN in the presence of the nucleic acid derivative using a regular refolding protocol.

### Design of an CpG-SAPN (Self-Assembling Protein Nanoparticle Encapsulating CpG)

A particular example of a CpG-SAPN according to the invention is the following construct “DEDDLI-RR”, corresponding to formula (I) with the sequence

(SEQ ID NO: 1)
MGDKHHHHHHHHHHKDGSDKGSWEEWNARWDEWENDWNDW
REDWQAWRDDWARWRATWRRGRLLSRLERLERRNEELRRL
LQLIRHENRMVLQFVRALSMQNAELERRLEELARGMAQVI
NTNSLSLLTQNNLNRSQSALGTAIERLSSGLRINSARDDA
AGQAIANRFTANIRGLTQASRNANDGISIAQTTEGALNEI
NNNLQRVRELAVQSANSTNSQSDLDSIQAEITQRLNEIDR
VSGQTQFNGVRVLAQDNTLTIQVGANDGETIDIDLRQINS
QTLGLDQLNVQQKYKDGDKGDDKTENPLQRIDAALAQVDA
LRSDLGAVONRENSAITNLGNTVNNLSEARSRIEDSDYAT
EVSNMSRAQILQQAGTSVLAQANQVPQNVLSLLR

This is a construct composed of the following partial structures:

X1:
(SEQ ID NO: 2)
MGDKHHHHHHHHHHKDGSDKGS
ND1:
(SEQ ID NO: 3)
WEEWNARWDEWENDWNDWREDWQAWRDDWARWRATW
L1:
(SEQ ID NO: 4)
RRGR
ND2:
(SEQ ID NO: 5)
LLSRLERLERRNEELRRLLQLIRHENRMVLQFVRALSMQN
AELERRLEEL
Y1:
(SEQ ID NO: 6)
ARGMAQVINTNSLSLLTQNNLNRSQSALGTAIERLSSGLR
INSARDDAAGQAIANRFTANIRGLTQASRNANDGISIAQT
TEGALNEINNNLQRVRELAVQSANSTNSQSDLDSIQAEIT
QRLNEIDRVSGQTQFNGVRVLAQDNTLTIQVGANDGETID
IDLRQINSQTLGLDQLNVQQKYKDGDKGDDKTENPLQRID
AALAQVDALRSDLGAVQNRFNSAITNLGNTVNNLSEARSR
IEDSDYATEVSNMSRAQILQQAGTSVLAQANQVPQNVLSL
LR

For ease of purification DEDDLI-RR starts with the sequence X1 as defined in formula (I):

(SEQ ID NO: 2)
MGDKHHHHHHHHHHKDGSDKGS

which contains a His-tag for nickel affinity purification and at the DNA level restriction sites for further sub-cloning (NcoI and BamHI).

For ND1 a pentamerization domain was chosen (m=5). The particular pentameric coiled coil is a novel modification of the tryptophan-zipper pentamerization domain (Liu, J., et al. Proc Natl Acad Sci USA 2004, 101(46): 16156-16161) with pdb-entry 1T8Z.

The original tryptophan-zipper pentamerization domain has the sequence

(SEQ ID NO: 7)
SSNAKWDQWSSDWQTWNAKWDQWSNDWNAWRSDWQAWKDDWARWNQRWDN
WAT

The modified coiled-coil sequence of the pentamerization domain used for DEDDLI-RR starts at position 13, ends at position 49 and contains sequence variations at the C-terminal end (RATW (SEQ ID NO:36) instead of NQRW (SEQ ID NO:37)) and for solubility purposes several charge modifications at non-core positions of the coiled-coil but keeping the heptad repeat pattern of the tryptophane residues at core positions as in the original sequence (SEQ ID NO:8). Also, the two lysine residues are changed to arginine residues to avoid coupling of hapten molecules to the pentameric coiled-coil. Coiled-coil core residues at positions aa(a) and aa(d) are indicated in bold and are underscored.

(SEQ ID NO: 3)
13-WEEWNARWDEWENDWNDWREDWQAWRDDWARWRATW-48

This sequence is extended then by the short linker L1 with the sequence RRGR (SEQ ID NO:4), to connect with the coiled-coil sequence ND2. L1 contains a flexible residue G (glycine) between the two coiled-coil parts of the nanoparticle. It contains three positively charged arginine amino acids that provide the ionic interaction with the negatively charged encapsulated nucleic acid.

L1 is followed by a second coiled-coil domain ND2 with the following sequence:

(SEQ ID NO: 5)
LLSRLERLERRNEELRRLLQLIRHENRMVLQFVRALSMQNAELERRLEEL

It is a de-novo designed coiled-coil aimed at forming a dimeric coiled coil with three core aa(a) positions occupied by asparagine residues, which favor dimeric coiled-coil formation. Coiled-coil core residues at positions aa(a) and aa(d) are indicated in bold and are underscored. It contains the pan DR binding CD4 epitope string ELRRLLQLIRHENRMVLQFVRALSMQNA (SEQ ID NO:7) which in itself contains the promiscuous CD4/CD8 epitope IRHENRMVL (SEQ ID NO:8) (Parida R. et al., Vaccine 2007, 25:7530-7539) corresponding to residues 173 to 181 of the matrix protein 1 of influenza A virus with the sequence ID BAA01449.1.

The segment Y1 has the following sequence:

(SEQ ID NO: 6)
ARGMAQVINTNSLSLLTQNNLNRSQSALGTAIERLSSGLRINSARDDAAG
QAIANRFTANIRGLTQASRNANDGISIAQTTEGALNEINNNLQRVRELAV
QSANSTNSQSDLDSIQAEITQRLNEIDRVSGQTQFNGVRVLAQDNTLTIQ
VGANDGETIDIDLRQINSQTLGLDQLNVQQKYKDGDKGDDKTENPLQRID
AALAQVDALRSDLGAVQNRENSAITNLGNTVNNLSEARSRIEDSDYATEV
SNMSRAQILQQAGTSVLAQANQVPQNVLSLLR

It contains the sequence ARG harboring a XmaI restriction site followed by a fragment of flagellin and is composed of the D0 and D1 domains of Salmonella typhimurium flagellin (as in U.S. Pat. No. 8,420,102), that is further modified such that the lysine side chains that are not surface exposed are mutated to arginines, while in the loop connecting the D0 and D1 domain of flagellin with the sequence DGDKGDDK (SEQ ID NO:9) four lysine residues are built-in for the purpose of covalently coupling hapten molecules such as nicotine, heroin, cocaine or the like. This loop is surface exposed.

The sequence

(SEQ ID NO: 10)
MAQVINTNSLSLLTQNNLNRSQSALGTAIERLSSGLRINSARDDAAGQAI
ANRFTANIRGLTQASRNANDGISIAQTTEGALNEINNNLQRVRELAVQSA
NSTNSQSDLDSIQAEITQRLNEIDRVSGQTQFNGVRVLAQDNTLTIQVGA
NDGETIDIDLRQINSQTLGLDQLNVQQKYK

Corresponds to residues 1 to 180 of P06175.2 of the flagellar biosynthesis protein FliC, in which residues 20, 42, 59, 136 and 161 are mutated from lysine to arginine, while residue 172 is mutated from threonine to glutamine to insert a MfeI restriction site at the DNA level.

The sequence

(SEQ ID NO: 11)
TENPLQRIDAALAQVDALRSDLGAVQNRENSAITNLGNTVNNLSEARSRI
EDSDYATEVSNMSRAQILQQAGTSVLAQANQVPQNVLSLLR

Corresponds to residues 403 to 493 of P06175.2, in which residue 409 is mutated from lysine to arginine. It contains also the mutations T419A, T446S and S447E.

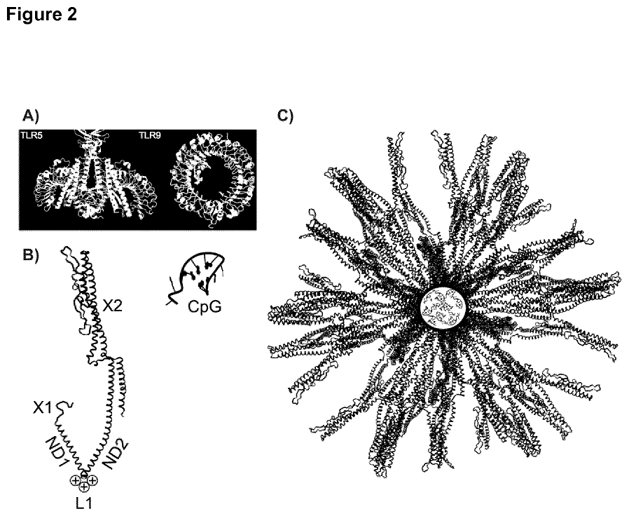

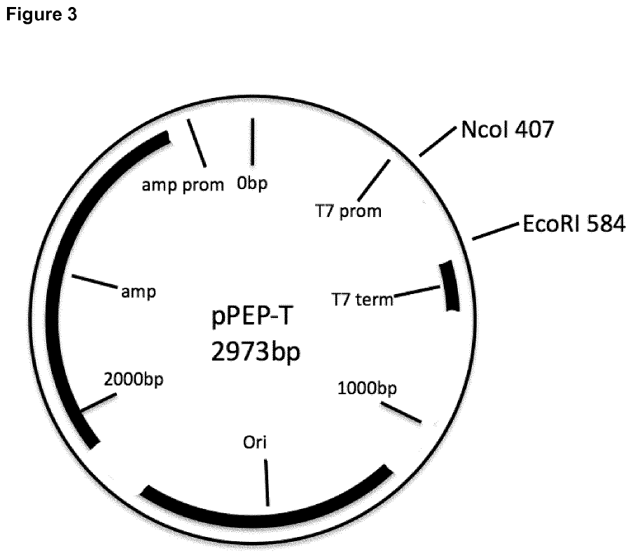

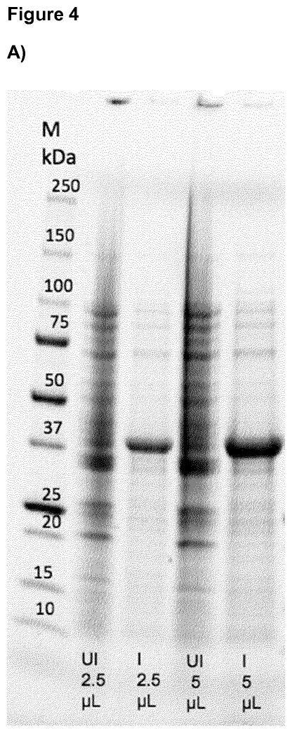

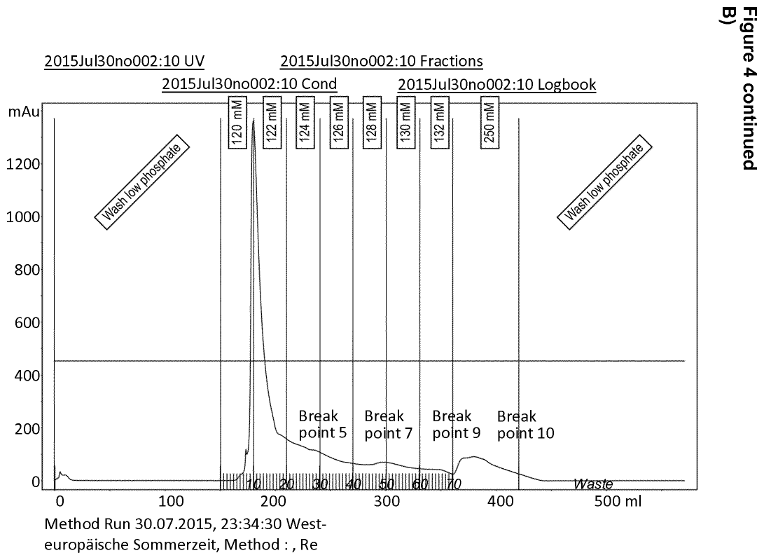

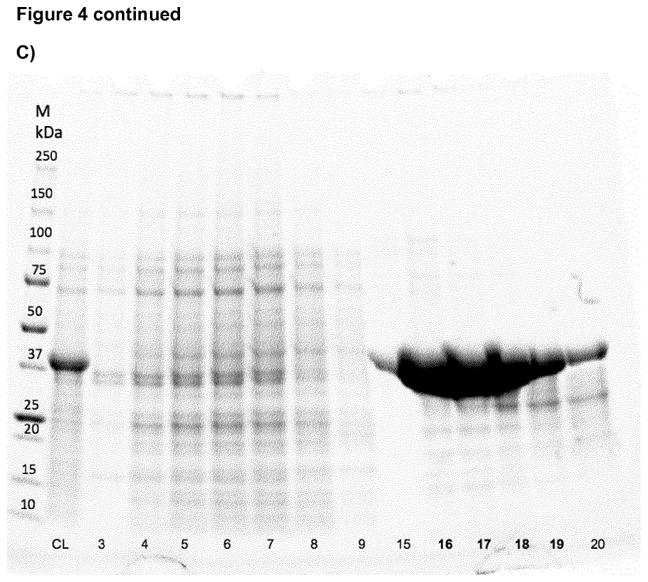

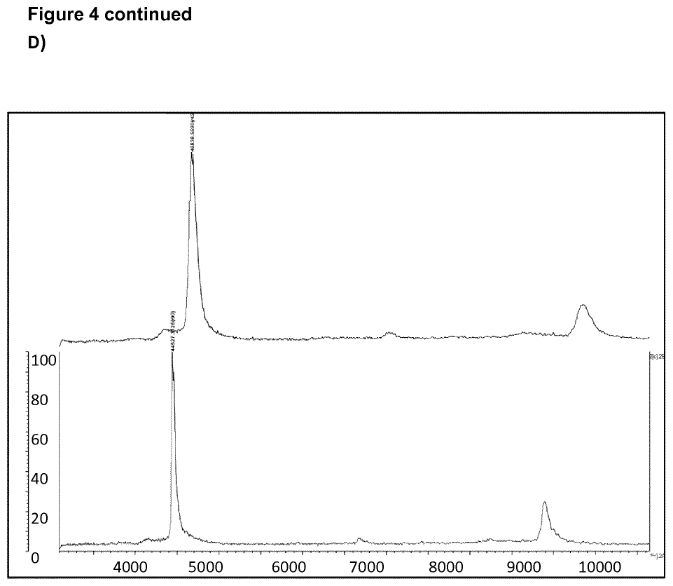

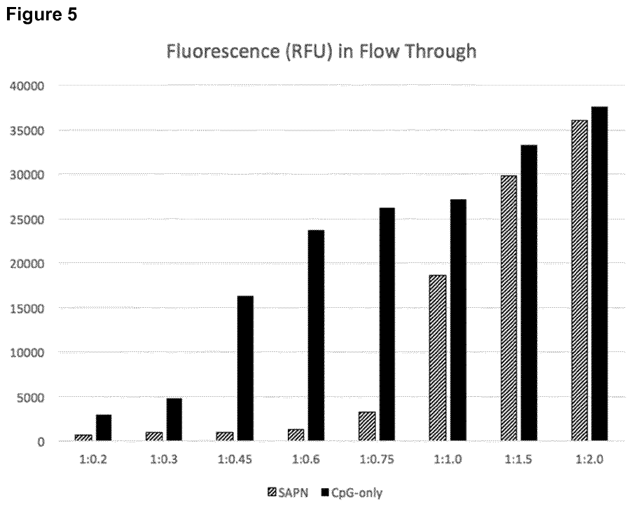

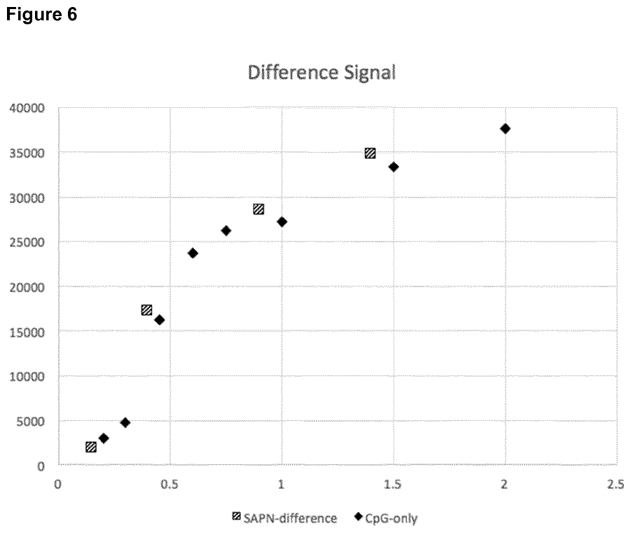

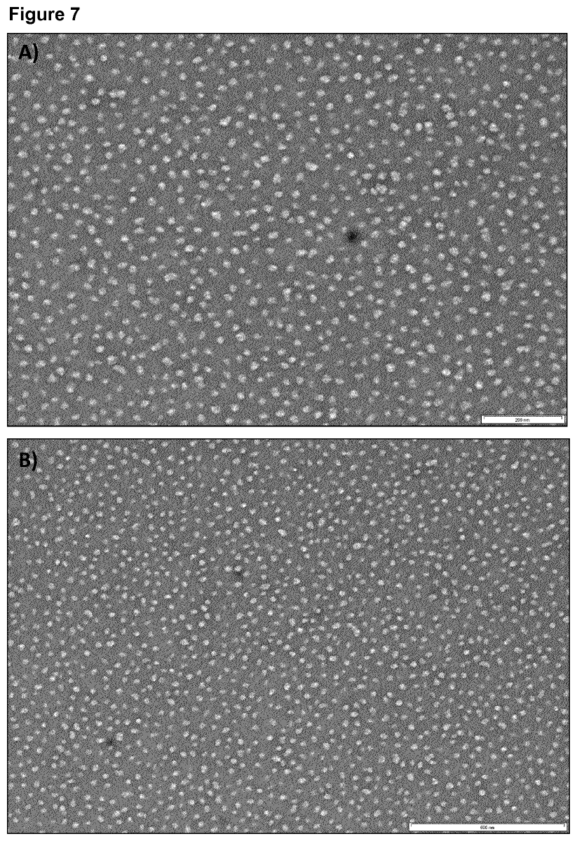

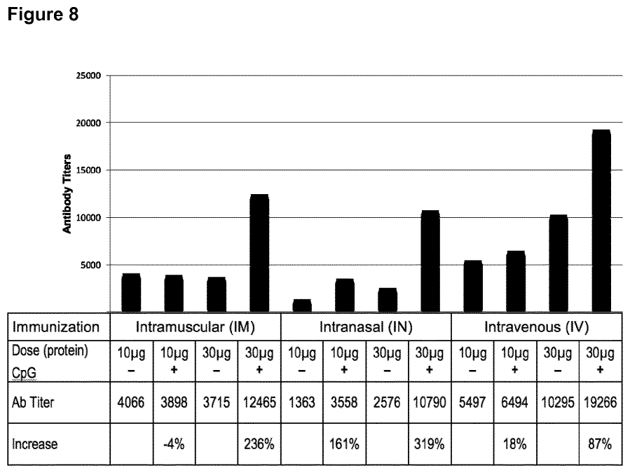

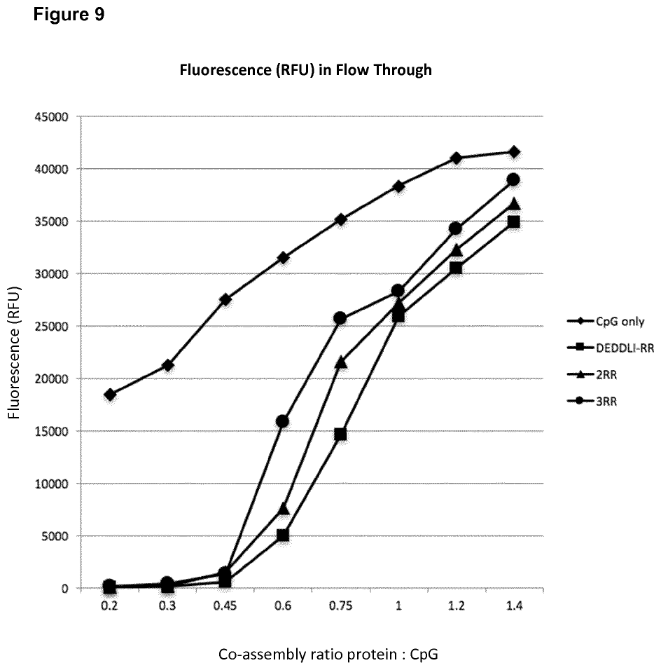

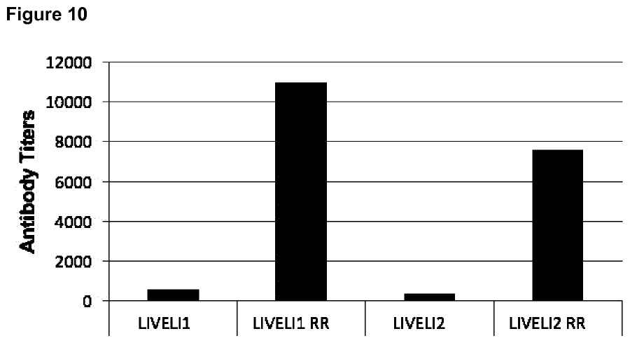

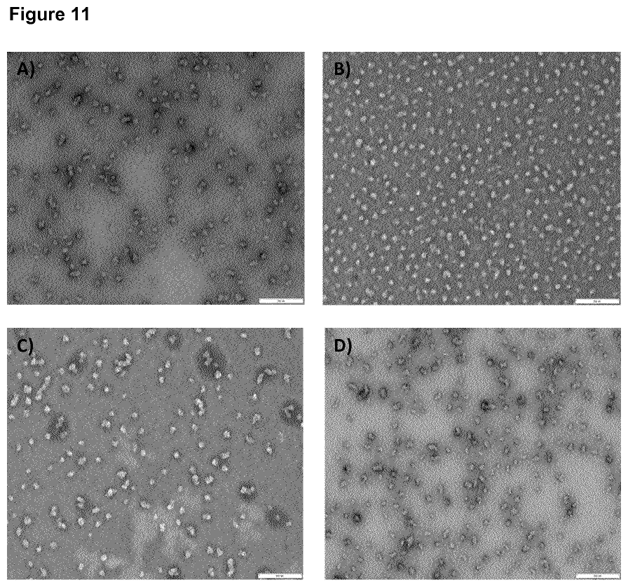

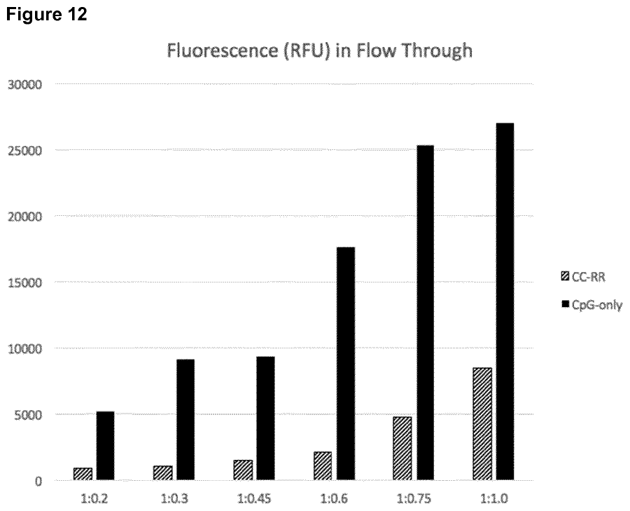

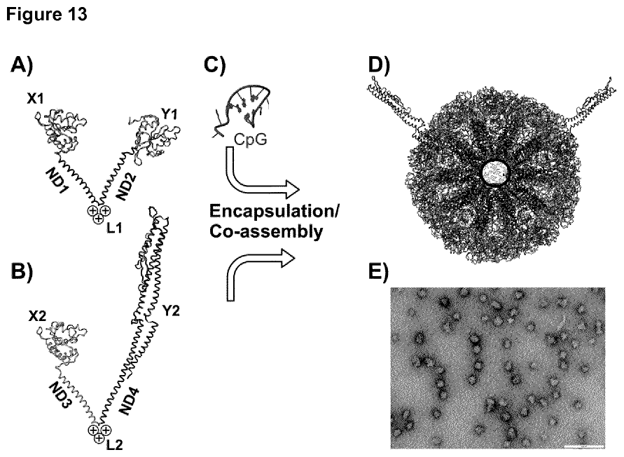

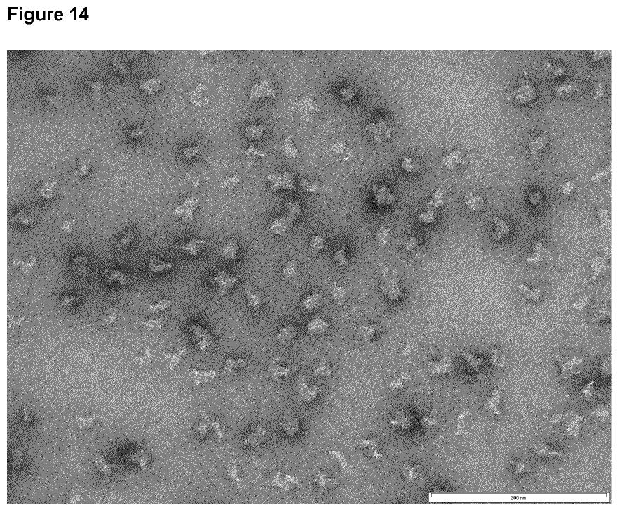

A model of DEDDLI-RR monomer is shown in FIG. 2 in its monomeric and icosahedral forms, assuming T=1 icosahedral symmetry. An EM picture of DEDDLI-RR is shown in FIG. 7.

### EXAMPLES

The following examples are useful to further explain the invention but in no way limit the scope of the invention.

### Example 1

### Molecular Cloning of DEDDLI-RR

The DNA coding for the nanoparticle constructs were prepared using standard molecular biology procedures. Plasmids containing the DNA coding for the protein sequence DEDDLI-RR

(SEQ ID NO: 12)
MGDKHHHHHHHHHHKDGSDKGSWEEWNARWDEWENDWNDWREDWQAWRDD
WARWRATWRRGRLLSRLERLERRNEELRRLLQLIRHENRMVLQFVRALSM
QNAELERRLEELARGMAQVINTNSLSLLTQNNLNRSQSALGTAIERLSSG
LRINSARDDAAGQAIANRFTANIRGLTQASRNANDGISIAQTTEGALNEI
NNNLQRVRELAVQSANSTNSQSDLDSIQAEITQRLNEIDRVSGQTQFNGV
RVLAQDNTLTIQVGANDGETIDIDLRQINSQTLGLDQLNVQQKYKDGDKG
DDKTENPLQRIDAALAQVDALRSDLGAVQNRENSAITNLGNTVNNLSEAR
SRIEDSDYATEVSNMSRAQILQQAGTSVLAQANQVPQNVLSLLR

was constructed by cloning into the NcoI/EcoRI restriction sites of the basic SAPN expression construct of pPEP-T (FIG. 3). The vaccine immunogen has been generated by covalently attaching the epitope nicotine to the carrier to the lysine residues by using the activated form of nicotine, NHS-nicotine.

The sequence of this construct with the architecture X1-ND1-L1-ND2-Y1 is described in detail above. Shortly, this construct is composed of a pentameric coiled-coil tryptophan zipper (ND1) linked to the dimeric de-novo designed coiled-coil (ND2) by the linker L1 with the sequence RRGR (SEQ ID NO: 4), that contains three positive charges between the last core position of the pentameric coiled coil and the first core position of the dimeric coiled coil. The sequence X1 at the N-terminus contains a His-tag and three hapten/nicotine binding sites (lysines), while the sequence Y1 contains a fragment of Salmonella typhimurium flagellin and is composed of the modified D0 and D1 domains of flagellin.

### Example 2

### Expression

The plasmids were transformed into Escherichia coli Tuner (DE3) cells, which were grown in Hyper Broth in presence of the antibiotic ampicillin (FIG. 4A). The pre-culture was grown at 28° C. The next day, a 1:500 dilution of pre-culture was inoculated into the expression 1 L culture and cells were grown at 37° C. with shaking in 5 L Erlenmeyer flask until an OD600 of about 0.8-0.9 was reached. The cell culture was then induced with IPTG (final concentration of 1 mM). After induction, the culture was grown under shaking at 37° C. for 3 hours. Then the cells were harvested by centrifugation at 4,000×g for 15 min. The cell pellet was stored at −20° C. The pellet was thawed on ice and suspended in a lysis buffer consisting of 6M Guanidine HCl, 300 mM NaH₂PO₄, 20 mM imidazole at pH 8.0.

### Example 3

### Purification

The following purification buffers were used:
1. High phosphate lysis buffer: 6M Guanidine HCl, 300 mM NaH₂PO₄, 20 mM imidazole pH 8.0
2. Low phosphate wash buffer: 6M Guanidine HCl, 20 mM NaH₂PO₄, 20 mM Imidazole, pH 8.0
3. Wash buffer for endotoxin removal: 10 mM Tris pH 8.0, 60% (v/v) Isopropanol
4. Elution buffer: low phosphate buffer 6M Guanidine HCl, 20 mM NaH₂PO₄ pH 8.0 with varying concentration of imidazole

Per gram of cell pellet, 5 to 10 mL volume of lysis buffer (6M GuHCl, 300 mM NaH₂PO₄, 20 mM imidazole pH 8.0) were used. 25 mL of lysis buffer was sonicated for 3 minutes on ice. The lysate was clarified using centrifugation at 15 K rpm for 45 min. After centrifugation, the cleared lysate was filtered using 0.45 μm filter (Sartorius) and purified on 2*5 mL His-trap HP affinity column.

First, the protein was bound to the column followed by washing steps according to the following scheme:
1. Equilibrating the column with high phosphate lysis buffer
2. Binding of filtered cleared lysate (CL) to the column
3. Wash 1 with high phosphate lysis buffer (5 column volumes)
4. Wash 2 with low phosphate buffer (5 column volumes)
5. Wash 3 with 60% isopropanol in 10 mM Tris pH 8.0 (10 column volumes)
6. Wash 4 with low phosphate buffer (15 column volumes)

Step 3 was performed to remove nucleic acid fragments while step 5 was used to remove endotoxin.

Thereafter, a stepwise elution with increasing imidazole concentrations of 120 to 132 mM imidazole was performed according to the following scheme (FIG. 4B):
1. Column wash with low phosphate buffer (5 column volumes)
2. Elution with 120 mM Imidazole (2 column volumes−fraction size 3 mL)
3. Elution with 122 mM Imidazole (2 column volumes−fraction size 3 mL)
4. Elution with 124 mM Imidazole (2 column volumes−fraction size 3 mL)
5. Elution with 126 mM Imidazole (2 column volumes−fraction size 3 mL)
6. Elution with 128 mM Imidazole (2 column volumes−fraction size 3mL)
7. Elution with 130 mM Imidazole (2 column volumes−fraction size 3 mL)
8. Elution with 132 mM Imidazole (2 column volumes−fraction size 3 mL)
9. Elution with 250 mM Imidazole (4 column volumes−fraction size 6 mL)
10. Column wash with low phosphate buffer (10 column volumes)

An SDS-PAGE of the purified DEDDLI-RR is shown in (FIG. 4C) and indicates a high yield of pure protein.

### Example 4

### Coupling of Nicotine

The pooled elution fractions of DEDDLI-RR and LIVELI1-RR were first incubated with 5 mM EDTA for at least one hour to remove any leached metal ions. This was followed by a dialysis using tangential flow filtration of the pooled elution fractions against the coupling buffer consisting of 6M Guanidine hydrochloride, 100 mM HEPES pH 7.2, 150 mM NaCl. A Spectra-Por 6-8 kDa cut-off membrane was used for dialysis.

12 mg of DEDDLI-RR at a concentration of 11.03 mg/mL was used for coupling correspond-ding to a volume of 1090 μL. The protein to NHS-nicotine molar ratio was 1:50. Hence, for this ratio the following amounts of protein and NHS-nicotine were used:
DEDDLI-RR: 0.267 μmoles (12 mg)
enantiopure NHS-nicotine: 13.35 μmoles

The coupling reaction was run in the dark (i.e. covered with an aluminum foil) at room temperature for 3 hours while stirring using a magnetic stirrer. After the coupling reaction, the sample was passed through PD minitrap G-25 prepacked columns to remove uncoupled NHS-nicotine and to buffer exchange to the pre-refolding buffer consisting of 8M Urea, 20 mM Tris pH 8.5, 150 mM NaCl and 10% Trehalose (FIG. 4D). The molecular masses of the construct before and after coupling were determined to be 44527.31 and 46838.55 Da, respectively, corresponding to an average of 8.9 nicotine molecules per proteins chains, i.e. all eight lysine side chains and the N-terminal amine are almost completely coupled with NHS-nicotine (FIG. 4D).

### Example 5

### Refolding

The final refolding buffer was prepared that contained either CpG for immunization experiments or fluorescent labeled CpG for the encapsulation studies. Mouse specific CpG (1826) with the sequence 5′-T*C*C*A*T*G*A*C*G*T*T*C*C*T*G*A*C*G*T*T-3′ (SEQ ID NO:13) in which the bases in the DNA backbone are connected by phosphorothioate bonds (indicated by the symbol *). The molecular weight of CpG 1826 is 6362.7 g/mol. The fluorescein-labeled CpG ODN1826F has a molecular weight of 6899.7 g/mol. ODN1826 is a Class B CpG sequence and contains two unmethylated CpG dinucleotides, which are highlighted in bold and underscore. Class B CpGs contain one or more CpG dinucleotides within a full phosphorothioate backbone that prevents rapid degradation. They strongly activate B cells but stimulate weakly IFN-α secretion.

Compared to mammalian DNA these unmethylated CpG dinucleotides exist at a 20-fold greater frequency in bacterial DNA. These motifs in this mouse-specific ODN1826 sequence are recognized by the mouse Toll-like receptor 9, which then leads to a strong immunostimulatory effect.

After quick refolding the final protein concentration is 0.05 mg/mL corresponding to 0.31 nmoles of protein. For the encapsulation experiments different molar ratios of protein to CpG were prepared. The following amounts of CpG were prepared for the different final refolding buffers: 0.06, 0.09, 0.14, 0.186, 0.233, 0.031, 0.451 and 0.62 nmoles corresponding to ratios of 1:0.2, 1:0.3, 1:0.45, 1:0.6, 1:0.75, 1:1, 1:1.5 and 1:2 of DEDDLI-RR:ODN1826F.

The protein in the pre-refolding buffer was then dropwise diluted into those final refolding buffer containing 20 mM Tris pH 8.0, 50 mM NaCl, 10% Trehalose containing different amounts of ODN1826. The quick refolding process was performed as follows: the final refolding buffer containing CpG was constantly kept stirring. The protein DEDDLI-RR was dropwise added to the final refolding buffer (with CpG) to initiate the refolding process. After addition of the protein the refolding process was allowed to continue for 5 minutes while constantly stirring.

### Example 6

### Encapsulation

After quick refolding, the total relative fluorescence units (RFU) was measured before filtration. Then, with the total volume of 300 μL of DEDDLI-RR:ODN1826F, a first filtration step was carried out by concentrating the protein by a factor of 2.5 fold (i.e. reducing the retentate from 300 μL to about 120 μL). The filtration step was carried out using 100 kDa cut-off centrifugal filter that allows free CpG to pass but retains the assembled SAPNs with the possibly encapsulated CpG. After the first filtration step, the RFU of the flow through and the retentate was measured.

TABLE 3
Fluorescence after encapsulation

| | | RFU | RFU after | |
|---|---|---|---|---|
| | Ratio/ | before | filtration | Flow |
| Sample | amount | filtration | Retentate | through |
| DEDDLI−RR:ODN1826F | 1:0.2 | 20213 | 19894 | 623 |
| | 1:0.3 | 24002 | 23916 | 1006 |
| | 1:0.45 | 26838 | 27099 | 1031 |
| | 1:0.6 | 27328 | 27226 | 1338 |
| | 1:0.75 | 30608 | 29400 | 3199 |
| | 1:1 | 37291 | 33636 | 18600 |
| | 1:1.5 | 44474 | 38639 | 29848 |
| | 1:2 | 47463 | 41840 | 36059 |
| ODN1826F only | 0.2 | 27357 | 15369 | 2981 |
| | 0.3 | 30792 | 23780 | 4803 |
| | 0.45 | 35611 | 26132 | 16263 |
| | 0.6 | 37880 | 27995 | 23710 |
| | 0.75 | 40254 | 28328 | 26177 |
| | 1 | 42871 | 34370 | 27173 |
| | 1.5 | 46935 | 41114 | 33326 |
| | 2 | 49291 | 42812 | 37622 |

It is important to be aware that due to fluorescence quenching the signal (RFU) of the fluorescence reading is highly non-linear with respect to the concentration. Therefore, successful encapsulation can best be observed in the column “Flow through” fraction. If CpG is encapsulated in the SAPN then it will not pass the filter and not give a signal in the “Flow through”. Up to an encapsulation ratio of 1:0.6 there is hardly any fluorescence detectable in the “Flow through” of the sample with the SAPN (DEDDLI-RR:ODN1826F) while in the sample without SAPN (ODN1826F-only) there is a rapid increase of the fluorescence intensity at these concentrations corresponding to lower encapsulation ratios 1:0.3, 1:0.45 and 1:0.6 (FIG. 5). At higher ratios no longer all fluorescence (i.e. CpG) can be retained by the SAPNs and hence the fluorescence signal increases significantly in the “Flow through” of the SAPN-containing samples (DEDDLI-RR:ODN1826F). This means that the SAPN can encapsulate an amount of CpG that corresponds to 0.6 times the molar ratio of protein chains, i.e. assuming a T1 icosahedral symmetry of the SAPN with 60 protein chains, roughly a total of 36 CpG molecules are encapsulated per nanoparticle.

Assuming a density of 1.8 g/cm³ of DNA in NaCl buffer, with a molecular weight of 6899.7 g/mol 36 molecules of ODN1826F occupy a sphere with a diameter of 7.6 nm. This is in very close agreement with the volume of the central cavity based on computer models of the SAPN.

If more CpG is added than what can be encapsulated by the SAPNs, then the additional CpG will pass through the membrane, leading to a increase in fluorescence intensity in the flow-through. This increase in fluorescence intensity in the flow-through due to the non-encapsulated CpG nicely correlates with the signal that is measured from CpG-only sample at the corresponding CpG concentrations (FIG. 6). Hence, the signal that is detected from the non-encapsulated CpG in the SAPN-containing sample is very similar to the signal from CpG-only sample and the concentration-dependent curves are almost overlapping (FIG. 6).

### Example 7

### Electron Microscopy

A transmission electron microscope analysis of the encapsulated DEDDLI-RR with ODN1826 shows very nice, non-aggregating nanoparticle formation (FIG. 7).

### Example 8

### Mouse Immunization Experiments

For the immunization experiments the molar ratio 1:0.6 of DEDDLI-RR:ODN1826 was used. After quick refolding, the solution containing refolded DEDDLI-RR with encapsulated CpG was dialyzed and filtered. The sample was then concentrated using a 100 kDa cut-off centrifugal filter (Millipore). A final sterile filtration step was done in the sterile hood using a 0.2 μm syringe filter (Sartorius).

Groups of five Balb/C mice each were immunized with two different doses of 10 μg and 30 μg protein, either with or without encapsulated CpG. The amount of CpG in those doses is 0.85 μg and 2.56 μg, respectively. Three injections each two weeks apart were given for the three different immunization protocols of intramuscular (IM), intranasal (IN) and intravenous (IV) injection. For each of the three immunization protocols a significant increase of the antibody titer can be observed when CpG is encapsulated in the SAPNs of the immunogen (Table 3, FIG. 8).
While for the IM immunization the 10 μg dose immunization shows the same strength of the immune response in terms of antibody titer with and without CpG, for the dose of 30 μg an increase of 236% can be observed for the sample with encapsulated CpG compared to the sample without CpG.
For the IN immunization the encapsulated CpG already increases the immune response at the lower dose of 10 μg of protein (corresponding to 0.85 μg of CpG) by 161%. For the 30 μg protein dose (2.56 μg CpG) the increase is as much as 319%.
While the immune response for the IM immunization is in general strongest, the influenza of the CpG is more moderate with increases of 18% and 87% for the low and high doses of 10 μg and 30 μg of protein (0.85 μg and 2.56 μg of CpG).

TABLE 3
Immune response with and without CpG encapsulation

| Immunization | Intramuscular (IM) | | | | Intranasal (IN) | | | | Intravenous (IV) | | | |
|---|---|---|---|---|---|---|---|---|---|---|---|---|
| Dose (protein) | 10 μg | 10 μg | 30 μg | 30 μg | 10 μg | 10 μg | 30 μg | 30 μg | 10 μg | 10 μg | 30 μg | 30 μg |
| CpG | − | + | − | + | − | + | − | + | − | + | − | + |
| Ab Titer | 4066 | 3898 | 3715 | 12465 | 1363 | 3558 | 2576 | 10790 | 5497 | 6494 | 10295 | 19266 |
| Increase | | −4% | | 236% | | 161% | | 319% | | 18% | | 87% |

### Example 9

### Testing Different Lengths and Overall Charges of Linker L1

It was expected that SAPNs with longer linkers L1 and carrying more positive charges than DEDDLI-RR with an overall charge of plus three would encapsulate negatively charged nucleic acids more efficiently, i.e. they could carry a bigger payload of nucleic acid. To test this assumption two new particles were designed that had a linker L1 of RRGRRGR (SEQ ID NO:14) and RRGRRGRRGR (SEQ ID NO:15), respectively. The length of the linker L1 of the first construct (dubbed 2RR) was seven amino acids with five positive charges (arginines), while the second construct (dubbed 3RR) had a nine amino acid long linker L1 with a total of seven positive charges (arginines).

The rationale for the modified linker is, that increasing the length of the linker L1 allows the two oligomerization domains ND1 and ND2 to be farther apart, thus increasing the size of the central cavity giving more space for cargo loading. Adding additional charges to the linker allows for better charge compensation between the protein and the negatively charged nucleic acid as the payload.

Since refolding behavior is critically dependent on the overall charges of the protein chain and hence of the overall charge of the particle itself, the additional positive charges in linker L1 of 2RR and 3RR were compensated by insertion of the negatively charged glutamic acids at the end of X1 right before the beginning of the pentamer ND1 and for 3RR by the change of an arginine residue close to the C-terminal end of the pentamer ND1 to a negatively charged aspartic acid. This keeps the overall charge of the protein chains of 2RR and 3RR at −7, the same as the overall charge of DEDDLI-RR. The sequences of 2RR and 3RR are then

(SEQ ID NO: 16)
MGDKHHHHHHHHHHKDGSDKGSEEWEEWNARWDEWENDWNDWREDWQAWR
DDWARWRATWRRGRRGRLLSRLERLERRNEELRRLLQLIRHENRMVLQFV
RALSMQNAELERRLEELARGMAQVINTNSLSLLTQNNLNRSQSALGTAIE
RLSSGLRINSARDDAAGQAIANRFTANIRGLTQASRNANDGISIAQTTEG
ALNEINNNLQRVRELAVQSANSTNSQSDLDSIQAEITQRLNEIDRVSGQT
QFNGVRVLAQDNTLTIQVGANDGETIDIDLRQINSQTLGLDQLNVQQKYK
DGDKGDDKTENPLQRIDAALAQVDALRSDLGAVQNRENSAITNLGNTVNN
LSEARSRIEDSDYATEVSNMSRAQILQQAGTSVLAQANQVPQNVLSLLR
and
(SEQ ID NO: 17)
MGDKHHHHHHHHHHKDGSDKGSEEWEEWNARWDEWENDWNDWREDWQAWR
DDWARWDATWRRGRRGRRGRLLSRLERLERRNEELRRLLQLIRHENRMVL
QFVRALSMQNAELERRLEELARGMAQVINTNSLSLLTQNNLNRSQSALGT
AIERLSSGLRINSARDDAAGQAIANRETANIRGLTQASRNANDGISIAQT
TEGALNEINNNLQRVRELAVQSANSTNSQSDLDSIQAEITQRLNEIDRVS
GQTQFNGVRVLAQDNTLTIQVGANDGETIDIDLRQINSQTLGLDQLNVQQ
KYKDGDKGDDKTENPLQRIDAALAQVDALRSDLGAVQNRFNSAITNLGNT
VNNLSEARSRIEDSDYATEVSNMSRAQILQQAGTSVLAQANQVPQNVLSL
LR

respectively. The calculated molecular weights of 2RR and 3RR are 45431.93 Da and Mw 45760.26 Da, respectively.

The two constructs were cloned, expressed and purified as in Examples 1, 2 and 3. Refolding and concomitant encapsulation was performed as described in Example 5 for DEDDLI-RR with slightly modified protein amounts used for the encapsulation ratios to account for the slightly different molecular weights compared to DEDDLI-RR.

The surprising finding, according to FIG. 9 is, that the longer and more positively charged linker of 2RR and 3RR did not allow more CpG to be encapsulated. While there is still very clearly CpG retained in the supernatant and not passing the filter into the Flow Through compared to the CpG-only sample, it is somewhat less than the encapsulation efficiency of DEDDLI-RR.

### Example 10

### Testing TLR9 Activation Without TLR5 Background Immunostimulation

In the construct DEDDLI-RR the D0/D1 domains of flagellin molecule activate the TLR5 to induce a strong immune response. This will overlay the immune response from CpG binding to TLR9. To test the immune response originating from TLR9 activation mainly, the TLR5 interaction site in DEDDLI-RR was modified to abrogate the interaction with the receptor. Arginine residues at the TLR5/flagellin interactions sites (Yoon S. I. et al., Science 2012, 335:859-64) were mutated to lysines. Also, the inflammasome interaction site at the C-terminal end of flagellin was mutated to disrupt the interaction with the inflammasome (Lightfield K. L. et al., Nat Immunol. 2008, 9:1171-8). The names of the two protein sequences are LIVELI1-RR and LIVELI2-RR and the corresponding sequences are then

(SEQ ID NO: 18)
MGDKHHHHHHHHHHKDGSDKGSWEEWNARWDEWENDWNDWREDWQAWRDD
WARWRATWRRGRLLSRLERLERRNEELRRLLQLIRHENRMVLQFVRALSM
QNAELERRLEELARGMAQVINTNSLSLLTQNNLNRSQSALGTAIERLSSG
LRINSARDDAAGQAIANRFTANIRGLTQASRNANDGISIAQTTEGALNEI
NNNLQKVKELAVQSANSTNSQSDLDSIQAEITQRLNEIDRVSGQTQFNGV
RVLAQDNTLTIQVGANDGETIDIDLRQINSQTLGLDQLNVQQKYKDGDKG
DDKTENPLQRIDAALAQVDALKSDLGAVQNRENSAITNLGNTVNNLSEAR
SRIEDSDYATEVSNMSRAQILQQAGTSVLAQANQVPQNVAAAAR
and
(SEQ ID NO: 19)
MGDKHHHHHHHHHHKDGSDKGSWEEWNARWDEWENDWNDWREDWQAWRDD
WARWRATWRRGRLLSRLERLERRNEELRRLLQLIRHENRMVLQFVRALSM
QNAELERRLEELARGMAQVINTNSLSLLTQNNLNKSQSALGTAIERLSSG
LRINSARDDAAGQAIANRFTANIKGLTQASRNANDGISIAQTTEGALNEI
NNNLQKVKELAVQSANSTNSQSDLDSIQAEITQRLNEIDRVSGQTQFNGV
KVLAQDNTLTIQVGANDGETIDIDLRQINSQTLGLDQLNVQQKYKDGDKG
DDKTENPLQKIDAALAQVDALKSDLGAVQNRENSAITNLGNTVNNLSEAR
SRIEDSDYATEVSNMSRAQILQQAGTSVLAQANQVPQNVAAAAR

In construct LIVELI1-RR the arginine residues 206, 208 and 322 of the construct DEDDLI-RR are mutated to lysines, while in LIVELI2-RR also arginine residues 135, 174, 251 and 310 of DEDDLI-RR are changed to lysines. Coupling the hapten nicotine at the primary amines of the lysine residues inserts bulky moieties at the interface between flagellin and TLR5, thus inhibiting complex formation and thus toll-like receptor based immunostimulation. In both construct LIVELI1-RR and LIVELI2-RR the inflammasome interaction site of the D0 domain of flagellin was modified to replace the residues 390 to 393 of DEDDLI-RR (LSLL) with four alanines (AAAA) (SEQ ID NO: 40). This modification will inhibit inflammasome activation of the two constructs LIVELI1-RR and LIVELI2-RR (Lightfield K. L. et al., Nat Immunol. 2008, 9:1171-8).

Since refolding without encapsulated CpG doesn't work so well for constructs with positively charged linkers a pair of constructs was prepared in which the positively charged linker RRGR (SEQ ID NO: 4) was replaced with the sequence MGGR (SEQ ID NO: 41), thus removing two of the three positive charges in the linker L1. Those constructs named LIVELI1 and LIVELI2 were used for the immunization without encapsulated CpG and had the overall sequences

(SEQ ID NO: 20)
MGDKHHHHHHHHHHKDGSDKGSWEEWNARWDEWENDWNDWREDWQAWRDD
WARWRATWMGGRLLSRLERLERRNEELRRLLQLIRHENRMVLQFVRALSM
QNAELERRLEELARGMAQVINTNSLSLLTQNNLNRSQSALGTAIERLSSG
LRINSARDDAAGQAIANRFTANIRGLTQASRNANDGISIAQTTEGALNEI
NNNLQKVKELAVQSANSTNSQSDLDSIQAEITQRLNEIDRVSGQTQFNGV
RVLAQDNTLTIQVGANDGETIDIDLRQINSQTLGLDQLNVQQKYKDGDKG
DDKTENPLQRIDAALAQVDALKSDLGAVQNRENSAITNLGNTVNNLSEAR
SRIEDSDYATEVSNMSRAQILQQAGTSVLAQANQVPQNVAAAAR
and
(SEQ ID NO: 21)
MGDKHHHHHHHHHHKDGSDKGSWEEWNARWDEWENDWNDWREDWQAWRDD
WARWRATWMGGRLLSRLERLERRNEELRRLLQLIRHENRMVLQFVRALSM
QNAELERRLEELARGMAQVINTNSLSLLTQNNLNKSQSALGTAIERLSSG
LRINSARDDAAGQAIANRFTANIKGLTQASRNANDGISIAQTTEGALNEI
NNNLQKVKELAVQSANSTNSQSDLDSIQAEITQRLNEIDRVSGQTQFNGV
KVLAQDNTLTIQVGANDGETIDIDLRQINSQTLGLDQLNVQQKYKDGDKG
DDKTENPLQKIDAALAQVDALKSDLGAVQNRENSAITNLGNTVNNLSEAR
SRIEDSDYATEVSNMSRAQILQQAGTSVLAQANQVPQNVAAAAR

The two pairs of constructs LIVELI1/LIVELI1-RR and LIVELI2/LIVELI2-RR were cloned, expressed and purified as in Examples 1, 2 and 3. Refolding and concomitant encapsulation was performed as described in Example 5 for DEDDLI-RR with slightly modified protein amounts used for the encapsulation ratios to account for the slightly different molecular weights compared to DEDDLI-RR. For the immunization experiments the molar ratio 1:0.6 of protein:ODN1826 was used. After quick refolding, the solution containing refolded nanoparticles with encapsulated CpG was dialyzed and filtered. The samples were then concentrated using a 100 kDa cut-off centrifugal filter (Millipore). A final sterile filtration step was done in the sterile hood using a 0.2 μm syringe filter (Sartorius).

Groups of five Balb/C mice each were immunized with a dose of 30 μg protein, either with (LIVELI1-RR and LIVELI2-RR) or without encapsulated CpG (LIVELI1 and LIVELI2). The amount of encapsulated CpG in the LIVELI1-RR and LIVELI2-RR doses is about 2.5 μg. Three injections each two weeks apart were given intramuscular in the immunization protocol. For both pairs of immunogens a very significant increase of the antibody titer can be observed when CpG is encapsulated in the SAPNs of the immunogen (FIG. 10). The antibody titer without ODN1826 for LIVELI1 and LIVELI2 immunogens were 576.3 and 367.6, respectively, while encapsulated CpG in LIVELI1-RR and LIVELI2-RR increased the antibody titer to 10958.0 and 7618.4, respectively, corresponding to a roughly twenty-fold increase.

### Example 11

### Malaria Vaccine by Co-Assembly with a Flagellin-Containing Protein Chain (CC-RR)

A further example of a CpG-SAPN according to the invention is the following construct “CC-RR”, in which two different protein chains are co-assembled, corresponding to formulas (I) and (II) with the sequences

(SEQ ID NO: 22)
MGHHHHHHHHHHTFRGNNGHNSSSSLYNGSQFIEQLNNSFTSAFLESQSM
NKIGDDLAETISNELVSVLQKNSPTFLESSFDIKSEVKKHAKSMLKELIK
VGLPSFENLVAENVKPPKVDPATYGIIVPVLTSLENKVETAVGAKVSDEI
WNYNSPDVSESEESLSDDFFDASGSAKFVAAWTLKAAASGSWERWNAKWD
EWRNDQNDWREDWQAWRDDWAYWTLTWRRGRLYSRLARIERRVEELRRLL
QLIRHENRMVLQFVRALSMQARRLEALIDYNKAALSKFKEDARGTFRGNN
GHNSSSSLYNGSQFIEQLNNSFTSAFLESQSMNKIGDDLAETISNELVSV
LQKNSPTFLESSFDIKSEVKKHAKSMLKELIKVGLPSFENLVAENVKPPK
VDPATYGIIVPVLTSLFNKVETAVGAKVSDEIWNYNSPDVSESEESLSDD
FFD

for formula (I) and

(SEQ ID NO: 23)
MGHHHHHHHHHHTFRGNNGHNSSSSLYNGSQFIEQLNNSFTSAFLESQSM
NKIGDDLAETISNELVSVLQKNSPTFLESSFDIKSEVKKHAKSMLKELIK
VGLPSFENLVAENVKPPKVDPATYGIIVPVLTSLENKVETAVGAKVSDEI
WNYNSPDVSESEESLSDDFFDASGSAKFVAAWTLKAAASGSWERWNAKWD
EWRNDQNDWREDWQAWRDDWAYWTLTWRRGRLYSRLARIERRVEELRRLL
QLIRHENRMVLQFVRALSMQARRLERRLEELARGMAQVINTNSLSLLTQN
NLNRSQSALGTAIERLSSGLRINSARDDAAGQAIANRFTANIRGLTQASR
NANDGISIAQTTEGALNEINNNLQRVRELAVQSANSTNSQSDLDSIQAEI
TQRLNEIDRVSGQTQENGVRVLAQDNTLTIQVGANDGETIDIDLRQINSQ
TLGLDQLNVQQKYKDGDKGDDKTENPLQRIDAALAQVDALRSDLGAVQNR
ENSAITNLGNTVNNLSEARSRIEDSDYATEVSNMSRAQILQQAGTSVLAQ
ANQVPQNVLSLLR

for formula (II)

The first construct corresponds to formula X1-ND1-L1-ND2-Y1 (I) with the following partial structures.

X1:
(SEQ ID NO: 24)
MGHHHHHHHHHHTFRGNNGHNSSSSLYNGSQFIEQLNNSFTSAFLESQSM
NKIGDDLAETISNELVSVLQKNSPTFLESSFDIKSEVKKHAKSMLKELIK
VGLPSFENLVAENVKPPKVDPATYGIIVPVLTSLFNKVETAVGAKVSDEI
WNYNSPDVSESEESLSDDFFDASGSAKFVAAWTLKAAASGS
ND1:
(SEQ ID NO: 25)
WERWNAKWDEWRNDQNDWREDWQAWRDDWAYWTLTW
L1:
(SEQ ID NO: 4)
RRGR
ND2:
(SEQ ID NO: 26)
LYSRLARIERRVEELRRLLQLIRHENRMVLQFVRALSMQARRL
Y1:
(SEQ ID NO: 27)
EALIDYNKAALSKFKEDARGTFRGNNGHNSSSSLYNGSQFIEQLNNSFTS
AFLESQSMNKIGDDLAETISNELVSVLQKNSPTFLESSFDIKSEVKKHAK
SMLKELIKVGLPSFENLVAENVKPPKVDPATYGIIVPVLTSLFNKVETAV
GAKVSDEIWNYNSPDVSESEESLSDDFFD

The second construct corresponds to formula X2-ND3-L2-ND4-Y2 (II) with the following partial structures.

X2:
(SEQ ID NO: 24)
MGHHHHHHHHHHTFRGNNGHNSSSSLYNGSQFIEQLNNSFTSAFLESQSM
NKIGDDLAETISNELVSVLQKNSPTFLESSFDIKSEVKKHAKSMLKELIK
VGLPSFENLVAENVKPPKVDPATYGIIVPVLTSLFNKVETAVGAKVSDEI
WNYNSPDVSESEESLSDDFFDASGSAKFVAAWTLKAAASGS
ND3:
(SEQ ID NO: 25)
WERWNAKWDEWRNDQNDWREDWQAWRDDWAYWTLTW
L2:
(SEQ ID NO: 4)
RRGR
ND4:
(SEQ ID NO: 26)
LYSRLARIERRVEELRRLLQLIRHENRMVLQFVRALSMQARRL
Y2:
(SEQ ID NO: 28)
ERRLEELARGMAQVINTNSLSLLTQNNLNRSQSALGTAIERLSSGLRINS
ARDDAAGQAIANRFTANIRGLTQASRNANDGISIAQTTEGALNEINNNLQ
RVRELAVQSANSTNSQSDLDSIQAEITQRLNEIDRVSGQTQFNGVRVLAQ
DNTLTIQVGANDGETIDIDLRQINSQTLGLDQLNVQQKYKDGDKGDDKTE
NPLQRIDAALAQVDALRSDLGAVQNRFNSAITNLGNTVNNLSEARSRIED
SDYATEVSNMSRAQILQQAGTSVLAQANQVPQNVLSLLR

In particular, the different fragments in these constructs are the following: X1 contains the His-tag (HHHHHHHHHH) (SEQ ID NO: 29), followed by the malarial antigen CeITOS (TFRGNNGHNSSSSLYNGSQFIEQLNNSFTSAFLESQSMNKIGDDLAETISNELVSVLQKNSP TFLESSFDIKSEVKKHAKSMLKELIKVGLPSFENLVAENVKPPKVDPATYGIIVPVLTSLFNKVE TAVGAKVSDEIWNYNSPDVSESEESLSDDFFD) (SEQ ID NO: 30) and the pan-DR binding epitope PADRE (AKFVAAWTLKAAA) (SEQ ID NO: 31) flanked and separated by peptide sequences that code for the restrictions sites NcoI, NheI and BamHI (MG, ASGS and SGS).

ND1 is a pentameric coiled coil derived from the tryptophane zipper (Liu J et al., Proc Natl Acad Sci USA 2004; 101(46):16156-61, pdb-entry 1T8Z, SEQ ID NO:7) with some charge modifications. It is similar to the ND1 domain of DEDDLI-RR with SEQ ID NO:3.

L1 is the same linker as in DEDDLI-RR with the sequence RRGR and SEQ ID NO:4.

ND2 is a coiled-coil domain with a very similar sequence as ND2 (SEQ ID NO:5) in the construct DEDDLI-RR also containing the promiscuous CD4/CD8 epitope IRHENRMVL (SEQ ID NO:8) (Parida R. et al., Vaccine 2007, 25:7530-7539) corresponding to residues 173 to 181 of the matrix protein 1 of influenza A virus with the sequence ID BAA01449.1.

Y1 starts with a sequence containing the CD4 epitope from the glycoprotein of Lymphocytic choriomeningitis mammarenavirus LIDYNKAALSKFKED (SEQ ID NO: 32) followed by a second copy of the malarial antigen CeITOS (TFRGNNGHNSSSSLYNGSQFIEQLNNSFTSAFLESQSMNKIGDDLAETISNELVSVLQKNSP TFLESSFDIKSEVKKHAKSMLKELIKVGLPSFENLVAENVKPPKVDPATYGIIVPVLTSLFNKVE TAVGAKVSDEIWNYNSPDVSESEESLSDDFFD) (SEQ ID NO: 30) flanked and se-parated by peptide sequences that code at the DNA level for the restriction sites XhoI and XmaI (LE and ARG). The XhoI restriction site is shared with the fragment ND2.

The only difference between the first and the second construct is the difference in the partial structures Y1 and Y2, i.e. the other fragments are identical between the two constructs, which means that X1 is equal to X2, ND1 is equal to ND3, L1 is equal to L2 and ND2 is equal to ND4. Therefore, the two constructs can be co-assembled as the coiled-coil oligomerization domains of the two constructs are the same. This is the concept that has been described in Patent WO 20151104352A1 in which a flagellin-containing protein chain is co-assembled with a B-cell epitope carrying protein chain.

Y2 of the second construct, in contrast to Y1 of the first construct, contains the D0 and D1 domains of flagellin. It starts with a small α-helical segment (ERRLEEL) (SEQ ID NO: 33) before the flagellin sequence that extends the coiled coil of ND4 a little further. Y2 is also flanked and separated by peptide sequences that code for the restrictions sites XhoI and XmaI (LE and ARG) with the XhoI restriction site being shared with the fragment ND4.

Co-assembly of the two constructs forms a SAPN that displays on both coiled coils the B-cell epitope CeITOS, while a small number of flagellin molecules are incorporated into the SAPN, depending on the co-assembly ratio between the first and the second construct. The positively charged linkers L1 and L2 are again located at the central cavity of the SAPN, thus allowing for ionic interactions with the negatively charged CpG. Again, CpG ODN1826 is encapsulated into the SAPNs during refolding.

The two constructs were cloned, expressed and purified as in Examples 1, 2 and 3. Refolding and concomitant encapsulation was performed as described in Example 5 for DEDDLI-RR with modified protein amounts used for the same encapsulation ratio of 1:0.6 (protein:CpG) to account for the different molecular weights compared to DEDDLI-RR. Similar to the DEDDLI-RR construct the encapsulation efficiency is about 1:0.6 for the ratio of protein chains to CpG ODN1826F molecules as evidenced by the retention rate in the fluorescence filtration experiments described above. CC-RR is able to retain the fluorescent ODN1826F molecule up to a co-assembly ratio of 1:0.6 (FIG. 12).

A figure describing the molecular architecture of the SAPNs, the process of the co-assembly/encapsulation procedure and an EM micrograph are shown in FIG. 13 for a co-assembly ratio of 58:2 of the first and second protein chain.

### Example 12

### HSV Mouse Immunogen “RR-SSIEF” with CD4 and CD8 Epitopes

As for the DEDDLI-RR construct (Example 1) the DNA coding for RR-SSIEF was prepared using standard molecular biology procedures. The plasmid containing the DNA coding for the protein sequence RR-SSIEF

(SEQ ID NO: 34)
MGDKHHHHHHHHHHKDGSDKGSWEEWNARWDEWENDWNDWREDWQAWR
DDWARWRATWRRGRLLSRLERLERRNEELRRLLQLIRHENRMVLQFVRAL
SMQNAELERRLEELARGMAQVINTNSLSLLTQNNLNRSQSALGTAIERLS
SGLRINSARDDAAGQAIANRFTANIRGLTQASRNANDGISIAQTTEGALN
EINNNLQRVRELAVQSANSTNSQSDLDSIQAEITQRLNEIDRVSGQTQFN
GVRVLAQDNTLTIQVGANDGETIDIDLRQINSQTLGLDQLNVQQAKFVAA
WTLKAAASSIEFARLQFDDTENPLQRIDAALAQVDALRSDLGAVQNRENS
AITNLGNTVNNLSEARSRIEDSDYATEVSNMSRAQILQQAGTSVLAQANQ
VPQNVLSLLR

was constructed by cloning into the NcoI/EcoRI restriction sites of the basic SAPN expression construct of pPEP-T (FIG. 3).

The sequence of this construct with the architecture X1-ND1-L1-ND2-Y1 is similar to the one of DEDDLI-RR described in detail above. Shortly, this construct is composed of a pentameric coiled-coil tryptophan zipper (ND1) linked to the dimeric de-novo designed coiled-coil (ND2) by the linker L1 with the sequence RRGR, that contains three positive charges between the last core position of the pentameric coiled coil and the first core position of the dimeric coiled coil. The sequence X1 at the N-terminus contains a His-tag, while the sequence Y1 contains a fragment of Salmonella typhimurium flagellin that is composed of the modified D0 and D1 domains of flagellin. The peptide sequence connecting the D0 and D1 domains of flagellin has the sequence QLNVQQAKFVAAWTLKAAASSIEFARLQFDD TENPLQ (SEQ ID NO: 35) between the restriction sites of MfeI and PstI. This connecting fragment contains the pan-DR binding CD4 epitope PADRE as well as the mouse-specific (haplotype H-2k) CD8 epitope SSIEFARL of the envelope glycoprotein B of Human alphaherpesvirus 2. The crystal structure of this peptide in complex with the MHC-I molecule is deposited in the Brookhaven database with entry code 1T0M.

To induce a Th1 immune response the Class A CpG ODN1585 was used instead of the Class B ODN1826. ODN1585 has the sequence 5′-ggGGTCAACGTTGAgggggg-3′ (SEQ ID NR:39) with bases in capital letters representing phosphodiester bonds while bases in lower case contain phosphorothioate bonds between bases.

The construct RR-SSIEF was cloned, expressed and purified as in Examples 1, 2 and 3. Refolding and concomitant encapsulation was performed as described in Example 5 for DEDDLI-RR with a slightly modified protein amount used for the encapsulation ratios to account for the slightly different molecular weight of RR-SSIEF compared to DEDDLI-RR and the different molecular weight of ODN1585 to ODN1826. For the immunization experiments the molar ratio 1:0.6 of protein:ODN1585 was used. After quick refolding, the solution containing refolded nanoparticles with encapsulated CpG was dialyzed and filtered. The samples were then concentrated using a 100 kDa cut-off centrifugal filter (Millipore). A final sterile filtration step was done in the sterile hood using a 0.2 μm syringe filter (Sartorius). A transmission electron microscope analysis of the encapsulated RR-SSIEF with ODN1585 shows very nice, non-aggregating nanoparticle formation (FIG. 14).

1. A composition for inducing an immune response in a subject comprising:
(a) a self-assembling protein nanoparticle (SAPN) consisting of a multitude of building blocks of formula (I)
X1-ND1-L1-ND2-Y1 (I),
consisting of a continuous chain comprising a coiled-coil oligomerization domain ND1, a linker L1, a coiled-coil oligomerization domain ND2 and further substituents X1 and Y1, wherein
ND1 is a coiled-coil oligomerization domain that comprises oligomers (ND1)ₘ of m subunits ND1,
ND2 is a coiled-coil oligomerization domain that comprises oligomers (ND2)ₙ of n subunits ND2,
m and n each is a figure between 2 and 10, with the proviso that m is not equal n and not a multiple of n, and n is not a multiple of m,
L1 is a peptide linker with an overall positive charge of at least +2 at physiological conditions,
X1 is absent or a peptide or protein sequence comprising 1 to 1000 amino acids that may be further substituted.
Y1 is absent or a peptide or protein sequence comprising 1 to 1000 amino acids that may be further substituted,
wherein the multitude of building blocks of formula (I) is optionally co-assembled with a multitude of building blocks of formula (II)
X2-ND3-L2-ND4-Y2 (II),
consisting of a continuous chain comprising a coiled-coil oligomerization domain ND3, a linker L2, a coiled-coil oligomerization domain ND4, and further substituents X2 and Y2, wherein
ND3 is a coiled-coil oligomerization domain that comprises oligomers (ND3)_{y} of y subunits ND3,
ND4 is a coiled-coil oligomerization domain that comprises oligomers (ND4)_{z} of z subunits ND4,
y and z each is a figure between 2 and 10, with the proviso that y is not equal z and not a multiple of z, and z is not a multiple of y, and wherein
either ND3 is identical to ND1, or ND4 is identical to ND2 or both ND3 and ND4 are identical to ND1 and ND2, respectively,
L2 is a peptide linker with an overall positive charge of at least +2 at physiological conditions, X2 is absent or a peptide or protein sequence comprising 1 to 1000 amino acids that may be further substituted
Y2 is absent or a peptide or protein sequence comprising 1 to 1000 amino acids that may be further substituted,
(b) an immunostimulatory substance, wherein said immunostimulatory substance is a nucleic acid derivative wherein said nucleic acid derivative is encapsulated into said SAPN.

2. The composition according to claim 1 wherein the peptide linker L1 and/or the peptide linker L2 independently from each other consists of at least four amino acids and has an overall positive charge of at least +3 at physiological conditions.

3. The composition according to claim 1 wherein the peptide linker L1 and/or the peptide linker L2 independently from each other comprises an amino acid sequence selected from the group consisting of the amino acid sequence as shown in SEQ ID NO:4, the amino acid sequence as shown in SEQ ID NO:12, the amino acid sequence as shown in SEQ ID NO: 14 and the amino acid sequence as shown in SEQ ID NO: 15.

4. The composition according to claim 1 wherein the nucleic acid derivative is selected from the group consisting of single-stranded DNA that contain a cytosine followed by a guanine wherein the cytosine nucleotide is unmethylated, single-stranded RNA from RNA viruses, double-stranded RNA from RNA viruses and polymeric complexes mimicking double-stranded RNA from RNA viruses.

5. The composition according to claim 1 wherein the nucleic acid derivative is a CpG oligodeoxynucleotide (CpG ODN) selected from the group consisting of Class A CpG ODN, Class B CpG ODN and Class C ODN.

6. The composition according to claim 1 wherein the nucleic acid derivative is a CpG oligodeoxynucleotide (CpG ODN) selected from the group consisting of the nucleotide acid sequence as shown in SEQ ID NO:13, SEQ ID NO:39, SEQ ID NO:42, SEQ ID NO:43, SEQ ID NO:44, SEQ ID NO:45, SEQ ID NO:46, SEQ ID NO:47, SEQ ID NO:48 and SEQ ID NO:49.

7. The composition according to claim 1 wherein the nucleic acid derivative is bound to the SAPN by ionic interactions.

8. The composition according to claim 1 wherein the molar ratio of the protein chain of the SAPN consisting of a multitude of building blocks of formula (I) and the nucleic acid derivative is about 1 to about 0.6.

9. The composition according to claim 1 wherein either ND1 and/or ND3 or ND2 and/or ND4 is a coiled coil selected from the group consisting of pentameric coiled coils, tetrameric coiled coils, trimeric coiled coils, and dimeric coiled coils.

10. The composition according to claim 1 wherein either ND1 and/or ND3 or ND2 and/or ND4 is a pentameric coiled coil selected from the group consisting of 4PN8, 4PND, 4WBA, 3V2N, 3V2P, 3V2Q, 3V2R, 4EEB, 4EED, 3MIW, 1MZ9, 1FBM, 1VDF, 2GUV, 2HYN, 1ZLL, and 1T8Z, or wherein either ND1 and/or ND3 or ND2 and/or ND4 is a pentameric coiled coil selected from the group consisting of 4PN8, 4PND, 4WBA, 3V2N, 3V2P, 3V2Q, 3V2R, 4EEB, 4EED, 3MIW, 1MZ9, 1FBM, 1VDF, 2GUV, 2HYN, 1ZLL, and 1T8Z which contains an amino acid modification and/or is shortened at either or both ends, wherein each coiled coil is indicated according to the pdb entry numbering of the RCSB Protein Data Bank (RCSB PDB).

11. The composition according to claim 1 wherein either ND1 and/or ND3 or ND2 and/or ND4 is a tetrameric coiled coil selected from the group consisting of 5D60, 5D5Y, 5AL6, 4WB4, 4BHV, 4C5Q, 4GJW, 4H7R, 4H8F, 4BXT, 4LTO, 4LTP, 4LTQ, 4LTR, 3ZDO, 3RQA, 3R4A, 3R4H, 3TSI, 3K4T, 3F6N, 2O6N, 2OVC, 2O1J, 2OK, 2AG3, 2CCE, 1YBK, 1U9F, 1U9G, 1U9H, 1USD, 1USE, 1UNT, 1UNU, 1UNV, 1UNW, 1UNX, 1UNY, 1UNZ, 1UO0, 1UO1, 1UO2, 1UO3, 1UO4, 1UO5, 1W5I, 1W5L, 1FE6, 1G1I, 1G1J, 1EZJ, 1RH4, and 1GCL, or wherein either ND1 and/or ND3 or ND2 and/or ND4 is a tetrameric coiled coil selected from the group consisting of 5D60, 5D5Y, 5AL6, 4WB4, 4BHV, 4C5Q, 4GJW, 4H7R, 4H8F, 4BXT, 4LTO, 4LTP, 4LTQ, 4LTR, 3ZDO, 3RQA, 3R4A, 3R4H, 3TSI, 3K4T, 3F6N, 2O6N, 2OVC, 2O1J, 2OK, 2AG3, 2CCE, 1YBK, 1U9F, 1U9G, 1U9H, 1USD, 1USE, 1UNT, 1UNU, 1UNV, 1UNW, 1UNX, 1UNY, 1UNZ, 1UO0, 1UO1, 1UO2, 1UO3, 1UO4, 1UO5, 1W5I, 1W5L, 1FE6, 1G1I, 1G1J, 1EZJ, 1RH4, and 1GCL which contains an amino acid modification and/or is shortened at either or both ends, wherein each coiled coil is indicated according to the pdb entry numbering of the RCSB Protein Data Bank (RCSB PDB).

12. The composition according to claim 1 wherein either ND1 and/or ND3 or ND2 and/or ND4 is a trimeric coiled coil selected from the group consisting of 5TOH, 5TOI1, 5K92, 5KB0, 5KB1, 5KB2, 5KKV, 5EFM, 2N64, 5ABS, 5IEA, 5APP, 5APQ, 5APS, 5APY, 5APZ, 5D5Z, 4YPC, 4YV3, 4CGB, 4CGC, 4CJD, 4R0R, 4UW0, 4P67, 4OXM, 3W8V, 3W92, 3W93, 412L, 4K8U, 4JBZ, 3VTQ, 4L1R, 4JDO, 4J4A, 4E52, 3VY1, 3ZMF, 3VU5, 3VU6, 2YNY, 2YNZ, 2YO0, 2YO1, 2YO2, 4G1A, 4GIF, 3TQ2, 4DZK, 4DZL, 4DZN, 3TE3, 3R48, 3SWF, 3SWY, 3PR7, 2YK0, 2YKP, 2YKQ, 3NTN, 3PP5, 3MKO, 3MGN, 3NWA, 3NWD, 3NWF, 3L35, 3L36, 3L37, 3M9B, 3M9D, 2X6P, 3LJM, 3AHA, 3H7X, 3H7Z, 3LT6, 3LT7, 3GJP, 2KP8, 3KPE, 2WPR, 2WPS, 2WPY, 2WPZ, 2WQ0, 2WQ1, 2WQ2, 2WQ3, 3HFC, 3HFE, 3HRN, 3HRO, 3H5F, 3H5G, 2WG5, 2WG6, 2W6B, 2JJL, 2VRS, 3EFG, 3DUZ, 2OT5, 2Z2T, 2QIH, 3BK6, 2O7H, 2R32, 2JGO, 2Q7C, 2Q3I, 2Q5U, 2IBL, 1ZV8, 1ZVB, 2FXP, 1WT6, 2AKF, 1TGG, 1SLQ, 1S9Z, 1PW9, 1PWB, 1M7L, 1GZL, 1KYC, 1KFM, 1KFN, 1J0, 1IJ1, 1IJ2, 1IJ3, 1HQJ, 1QU1, 1B08, 1CZQ, 1CUN, 1SVF, 1CE0, 1PIQ, 1AQ5, 1AVY, 1HTN, 1AA0, 1ZIJ, 1ZIM, 1COI, 1SWI, 1GCM, and 1HUP, or wherein either ND1 and/or ND3 or ND2 and/or ND4 is a trimeric coiled coil selected from the group consisting of 5TOH, 5TOI, 5K92, 5KB0, 5KB1, 5KB2, 5KKV, 5EFM, 2N64, 5ABS, 5IEA, 5APP, 5APQ, 5APS, 5APY, 5APZ, 5D5Z, 4YPC, 4YV3, 4CGB, 4CGC, 4CJD, 4R0R, 4UW0, 4P67, 4OXM, 3W8V, 3W92, 3W93, 4I2L, 4K8U, 4JBZ, 3VTQ, 4L1R, 4JDO, 4J4A, 4E52, 3VYI, 3ZMF, 3VU5, 3VU6, 2YNY, 2YNZ, 2YO0, 2YO1, 2YO2, 4G1A, 4GIF, 3TQ2, 4DZK, 4DZL, 4DZN, 3TE3, 3R48, 3SWF, 3SWY, 3PR7, 2YKO, 2YKP, 2YKQ, 3NTN, 3PP5, 3MKO, 3MGN, 3NWA, 3NWD, 3NWF, 3L35, 3L36, 3L37, 3M9B, 3M9D, 2X6P, 3LJM, 3AHA, 3H7X, 3H7Z, 3LT6, 3LT7, 3GJP, 2KP8, 3KPE, 2WPR, 2WPS, 2WPY, 2WPZ, 2WQ0, 2WQ1, 2WQ2, 2WQ3, 3HFC, 3HFE, 3HRN, 3HRO, 3H5F, 3H5G, 2WG5, 2WG6, 2W6B, 2JJL, 2VRS, 3EFG, 3DUZ, 2OT5, 2Z2T, 2QIH, 3BK6, 2O7H, 2R32, 2JGO, 2Q7C, 2Q3I, 2Q5U, 2IBL, 1ZV8, 1ZVB, 2FXP, 1WT6, 2AKF, 1TGG, 1SLQ, 1S9Z, 1PW9, 1PWB, 1M7L, 1GZL, 1KYC, 1KFM, 1KFN, 1J0, 1IJ1, 1IJ2, 1IJ3, 1HQJ, 1QU1, 1B08, 1CZQ, 1CUN, 1SVF, 1CE0, 1PIQ, 1AQ5, 1AVY, 1HTN, 1AA0, 1ZIJ, 1ZIM, 1COI, 1SWI, 1GCM, and 1HUP which contains an amino acid modification and/or is shortened at either or both ends, wherein each coiled coil is indicated according to the pdb entry numbering of the RCSB Protein Data Bank (RCSB PDB).

13. The composition according to claim 1 wherein either ND1 and/or ND3 or ND2 and/or ND4 is a dimeric coiled coil selected from the group consisting of 5M97, 5M9E, 5FIY, 5F4Y, 5D3A, 5HMO, 5EYA, 5IX1, 5IX2, 5JHF, 5JVM, 5JVP, 5JVR, 5JVS, 5JVU, 5JX1, 5FCN, 5HHE, 2N9B, 4ZRY, 4Z6Y, 4YTO, 4Z13, 5AJS, 5F3K, 5F5R, 5HUZ, 5DJN, 5DJO, 5CHX, 5CJ0, 5CJ1, 5CJ4, 5C9N, 5CFF, 4WHV, 3WUT, 3WUU, 3WUV, 4ZQA, 4XA3, 4XA4, 4PXJ, 4YVC, 4YVE, 5BML, 5AL7, 4WOT, 4CG4, 5AMO, 4WII, 4WIK, 4RSJ, 4CFG, 4R3Q, 4WID, 4CKG, 4CKH, 4NSW, 4W7P, 4QQ4, 4OJK, 4TL1, 40H9, 4LPZ, 4Q62, 4L2W, 4M3L, 4CKM, 4CKN, 4N6J, 4LTB, 4LRZ, 2MAJ, 2MAK, 4NAD, 4HW0, 4BT8, 4BT9, 4BTA, 4HHD, 4M8M, 4J3N, 4L6Q, 4C1A, 4C1B, 4GDO, 4BWK, 4BWP, 4BWX, 4HU5, 4HU6, 4L9U, 4G0U, 4G0V, 4G0W, 4L3I, 4G79, 4GEU, 4GEX, 4GFA, 4GFC, 4BL6, 4JMR, 4JNH, 2YMY, 4HAN, 3VMY, 3VMZ, 3VN0, 4ABX, 3W03, 2LW9, 4DZM, 4ETO, 3TNU, 3THF, 4E8U, 3VMX, 4E61, 3VEM, 3VBB, 4DJG, 3TV7, 3STQ, 3V8S, 3Q8T, 3U1C, 3QH9, 3AZD, 3ONX, 3OKQ, 3QX3, 3SJA, 3SJB, 3SJC, 2L2L, 3QFL, 3QKT, 2XV5, 2Y3W, 3Q0X, 3AJW, 3NCZ, 3NI0, 2XU6, 3M91, 3NMD, 3LLL, 3LX7, 3ME9, 3MEU, 3MEV, 3ABH, 3ACO, 3IAO, 3HLS, 2WMM, 3A6M, 3A7O, 2WVR, 3ICX, 3ID5, 3ID6, 3HNW, 3I1G, 2K6S, 3GHG, 3G1E, 2W6A, 2V51, 3ERR, 3E1R, 2VY2, 2ZR2, 2ZR3, 3CL3, 3D9V, 2Z17, 2JEE, 3BBP, 3BAS, 3BAT, 2QM4, 2V71, 2NO2, 2PON, 2V0O, 2DQ0, 2DQ3, 2Q2F, 2NRN, 2E7S, 2H9V, 2FXM, 2HJD, 2GZD, 2GZH, 2FV4, 2F2U, 2EUL, 2ESM, 2ETK, 2ETR, 1ZXA, 1YIB, 1YIG, 1XSX, 1RFY, 1UO1, 1XJA, 1T3J, 1T6F, 1R7J, 1UII, 1PL5, 1S1C, 1P9I, 1R48, 1URU, 1OV9, 1UIX, 1NO4, 1NYH, 1MV4, 1LR1, 1L8D, 1LJ2, 1KQL, 1GXK, 1GXL, 1GK6, 1JR5, 1GMJ, 1JAD, 1JCH, 1JBG, 1JTH, 1JY2, 1JY3, 1IC2, 1HCl, 1HF9, 1HBW, 1FXK, 1D7M, 1QUU, 1CE9, 2A93, 1BM9, 1A93, 1TMZ, 2AAC, 1ZII, 1ZIK, 1ZIL, 2ARA, 2ARC, 1JUN, 1YSA, and 2ZTA, or wherein either ND1 and/or ND3 or ND2 and/or ND4 is a dimeric coiled coil selected from the group consisting of 5M97, 5M9E, 5FIY, 5F4Y, 5D3A, 5HMO, 5EYA, 5IX1, 5IX2, 5JHF, 5JVM, 5JVP, 5JVR, 5JVS, 5JVU, 5JX1, 5FCN, 5HHE, 2N9B, 4ZRY, 4Z6Y, 4YTO, 4ZI3, 5AJS, 5F3K, 5F5R, 5HUZ, 5DJN, 5DJO, 5CHX, 5CJ0, 5CJ1, 5CJ4, 5C9N, 5CFF, 4WHV, 3WUT, 3WUU, 3WUV, 4ZQA, 4XA3, 4XA4, 4PXJ, 4YVC, 4YVE, 5BM L, 5AL7, 4WOT, 4CG4, 5AMO, 4WII, 4WIK, 4RSJ, 4CFG, 4R3Q, 4WID, 4CKG, 4CKH, 4NSW, 4W7P, 4QQ4, 4OJK, 4TL1, 4OH9, 4LPZ, 4Q62, 4L2W, 4M3L, 4CKM, 4CKN, 4N6J, 4LTB, 4LRZ, 2MAJ, 2MAK, 4NAD, 4HW0, 4BT8, 4BT9, 4BTA, 4HHD, 4M8M, 4J3N, 4L6Q, 4C1A, 4C1B, 4GDO, 4BWK, 4BWP, 4BWX, 4HU5, 4HU6, 4L9U, 4G0U, 4G0V, 4G0W, 4L3I, 4G79, 4GEU, 4GEX, 4GFA, 4GFC, 4BL6, 4JMR, 4JNH, 2YMY, 4HAN, 3VMY, 3VMZ, 3VNO, 4ABX, 3W03, 2LW9, 4DZM, 4ETO, 3TNU, 3THF, 4E8U, 3VMX, 4E61, 3VEM, 3VBB, 4DJG, 3TV7, 3STQ, 3V8S, 3Q8T, 3U1C, 3QH9, 3AZD, 3ONX, 3OKQ, 3QX3, 3SJA, 3SJB, 3SJC, 2L2L, 3QFL, 3QKT, 2XV5, 2Y3W, 3QOX, 3AJW, 3NCZ, 3NI0, 2XU6, 3M91, 3NMD, 3LLL, 3LX7, 3ME9, 3MEU, 3MEV, 3ABH, 3ACO, 3IAO, 3HLS, 2WMM, 3A6M, 3A7O, 2WVR, 3ICX, 3ID5, 3ID6, 3HNW, 3I1G, 2K6S, 3GHG, 3G1E, 2W6A, 2V51, 3ERR, 3E1R, 2VY2, 2ZR2, 2ZR3, 3CL3, 3D9V, 2Z17, 2JEE, 3BBP, 3BAS, 3BAT, 2QM4, 2V71, 2NO2, 2PON, 2V0O, 2DQ0, 2DQ3, 2Q2F, 2NRN, 2E7S, 2H9V, 2FXM, 2HJD, 2GZD, 2GZH, 2FV4, 2F2U, 2EUL, 2ESM, 2ETK, 2ETR, 1ZXA, 1YIB, 1YIG, 1XSX, 1RFY, 1UO1, 1XJA, 1T3J, 1T6F, 1R7J, 1UII, 1PL5, 1S1C, 1P9I, 1R48, 1URU, 1OV9, 1UIX, 1NO4, 1NYH, 1MV4, 1LR1, 1L8D, 1LJ2, 1KQL, 1GXK, 1GXL, 1GK6, 1JR5, 1GMJ, 1JAD, 1JCH, 1JBG, 1JTH, 1JY2, 1JY3, 1IC2, 1HCl, 1HF9, 1HBW, 1FXK, 1D7M, 1QUU, 1CE9, 2A93, 1BM9, 1A93, 1TMZ, 2AAC, 1ZII, 1ZIK, 1ZIL, 2ARA, 2ARC, 1JUN, 1YSA, and 2ZTA, which contains an amino acid modification and/or is shortened at either or both ends, wherein each coiled coil is indicated according to the pdb entry numbering of the RCSB Protein Data Bank (RCSB PDB).

14. The composition according to claim 1 wherein the multitude of building blocks of formula (I) is co-assembled with a multitude of building blocks of formula (II) and the co-assembled SAPN comprising a multitude of building blocks of formula (I) and a multitude of building blocks of formula (II) has a co-assembly ratio of about 48 to about 59 of the continuous chain comprising a building block of formula (I) to about 1 to about 12 of the continuous chain comprising a building block of formula (II).

15. The composition according to claim 1 for use in a method of vaccinating a human or non-human animal, the method comprising administering an effective amount of said composition to a human or non-human animal in need of such vaccination.

16. The composition according to claim 2 wherein the nucleic acid derivative is selected from the group consisting of single-stranded DNA that contain a cytosine followed by a guanine wherein the cytosine nucleotide is unmethylated, single-stranded RNA from RNA viruses, double-stranded RNA from RNA viruses and polymeric complexes mimicking double-stranded RNA from RNA viruses.

17. The composition according to claim 2 wherein the nucleic acid derivative is a CpG oligodeoxynucleotide (CpG ODN) selected from the group consisting of Class A CpG ODN, Class B CpG ODN and Class C ODN.

18. The composition according to claim 2 wherein the nucleic acid derivative is a CpG oligodeoxynucleotide (CpG ODN) selected from the group consisting of the nucleotide acid sequence as shown in SEQ ID NO:13, SEQ ID NO:39, SEQ ID NO:42, SEQ ID NO:43, SEQ ID NO:44, SEQ ID NO:45, SEQ ID NO:46, SEQ ID NO:47, SEQ ID NO:48 and SEQ ID NO:49.

19. The composition according to claim 2 wherein the nucleic acid derivative is bound to the SAPN by ionic interactions.

20. The composition according to claim 2 wherein the molar ratio of the protein chain of the SAPN consisting of a multitude of building blocks of formula (I) and the nucleic acid derivative is about 1 to about 0.6.
